(19) United States
(12) Patent Application Publication
(10) Pub. No.: US 2025/0320248 A1
(43) Pub. Date: Oct. 16, 2025

(54) MENAQUINONE-BINDING COMPOUNDS AND METHODS OF USE THEREOF

(71) Applicant: The Rockefeller University, New York, NY (US)

(72) Inventors: Sean Brady, New York, NY (US); Lei Li, New York, NY (US); Bimal Koirala, New York, NY (US)

(21) Appl. No.: 18/702,954

(22) PCT Filed: Oct. 21, 2022
(86) PCT No.: PCT/US2022/078508

§ 371 Date: Apr. 19, 2024

Related U.S. Application Data
(60) Provisional application No. 63/270,804, filed on Oct. 22, 2021.

(51) Int. Cl. C07K 7/06; A61K 38/00; A61P 31/04; C07K 7/08; C12P 21/02

(52) U.S. Cl. CPC C07K 7/06; A61P 31/04; C07K 7/08; C12P 21/02; A61K 38/00

(57) ABSTRACT

The present invention provides methods, compositions, and articles of manufacture useful for treatment of multidrug-resistant pathogens and related conditions. The present invention provides compositions and methods incorporating and utilizing menaquinone-binding compounds or derivatives or variants thereof.

### CROSS-REFERENCE TO RELATED APPLICATIONS

This application claims priority to and the benefit of U.S. Provisional Application No. 63/270,804, filed Oct. 22, 2021, the disclosure of which is incorporated herein by reference in its entirety.

### STATEMENT OF GOVERNMENT FUNDING

This invention was made with government support under 1U19AI142731 and 5R35GM122559 awarded by the National Institutes of Health. The government has certain rights in the invention.

### BACKGROUND OF THE INVENTION

Antimicrobial resistance presents a major and growing healthcare problem and contributes annually to ˜700,000 deaths around the world (Wellcome Trust and UK Government. Review on antimicrobial resistance—tackling drug-resistant infections globally: Final report and recommendations, 2016, Wellcome Trust and UK Government; de Kraker et al., 2016, PLoS Med. 13, e1002184). The widespread emergence of multidrug-resistant (MDR) pathogens necessitates the development of in vivo active antibiotics that differ in mode of action from those that are currently in clinical use (Brown et al., 2016, Nature 529, 336-343; Niu et al., 2019, Trends Biochem. Sci. 44, 961-972; Lewis et al., 2020, Cell 181, 29-45). In the majority of anaerobic and Gram-positive bacteria, menaquinone (MK) plays an important role in electron transport (Johnston et al., 2020, Curr. Opin. Struct. Biol. 65, 33-41). Humans are not capable of producing MK, making it an appealing target for antibiotic development (Boersch et al., 2018, RSC Adv. 8, 5099-5105). Historically, inhibition of MK biosynthesis by synthetic small molecules has been the predominant mode of action explored to develop mechanistically novel antibiotics (Boersch et al., 2018, RSC Adv. 8, 5099-5105; Paudel et al., 2016, Drug Discov. Ther. 10, 123-128; Le et al., 2020, Nat. Chem. 12, 145-158). Three closely related nonribosomal peptide synthetase (NRPS) derived bacterial cyclic lipopeptides (lysocin E, WAP-8294A2 and WBP-29479A1) have been shown to kill bacteria by binding directly to MK to induce membrane disruption and rapid cell lysis (Hamamoto et al., 2015, Nat. Chem. Biol. 11, 127-133; Itoh et al., 2018, J. Org. Chem. 83, 6924-6935; Sang et al., 2019, Org. Lett. 21, 6432-6436). Recently, Santiago et al. reported that lysocin E also binds to lipid II, a precursor for bacterial cell wall synthesis (Santiago et al., 2018, Nat. Chem. Biol. 14, 601-608). Although WAP-8294A2 (lotilibcin) progressed to phase I clinical trials, a dearth of additional chemical entities that can induce antibiosis through MK binding, has hindered the successful therapeutic development of this mechanistically interesting class of antibiotics (Butler et al., 2013, J. Antibiot. 66, 571-591).

Thus, there is a need in the art for new compositions and methods for treating infections. The present invention satisfies the need in the art.

### SUMMARY OF THE INVENTION

The present invention provides a compound comprising the amino acid sequence (X^{A})ₐG(X^{B})_{b}L(X^{C})_{c}W(X^{D})_{d}.

In some embodiments, each occurrence of X^{A}, X^{B}, X^{c}, and X^{D} is independently selected from a natural amino acid, functionalized natural amino acid, unnatural amino acid, functionalized unnatural amino acid, or any combination thereof.

In some embodiments, each occurrence of a, b, c, and d is independently an integer from 0 to 100. In some embodiments, each occurrence of a, b, c, and d is independently an integer from 0 to 10.

In some embodiments, the compound is a cyclic compound.

In some embodiments, the amino acid sequence (X^{A})ₐG(X^{B})_{b}L(X^{C})_{c}W(X^{D})_{d} comprises at least one amino acid sequence selected from at least one amino acid sequence, or a fragment thereof, selected from FIG. 1; at least one amino acid sequence, or a fragment thereof, selected FIG. 5; at least one amino acid sequence, or a fragment thereof, selected from FIG. 7; at least one amino acid sequence, or a fragment thereof, selected from FIG. 12; at least one amino acid sequence, or a fragment thereof, selected from FIG. 13; at least one amino acid sequence, or a fragment thereof, selected from FIG. 15; at least one amino acid sequence, or a fragment thereof, selected from FIG. 16; the amino acid sequence GXLXXXW; or any combination thereof.

In some embodiments, each occurrence of X is independently selected from a natural amino acid, functionalized natural amino acid, unnatural amino acid, functionalized unnatural amino acid, or any combination thereof.

In some embodiments, the compound is a compound having the structure of

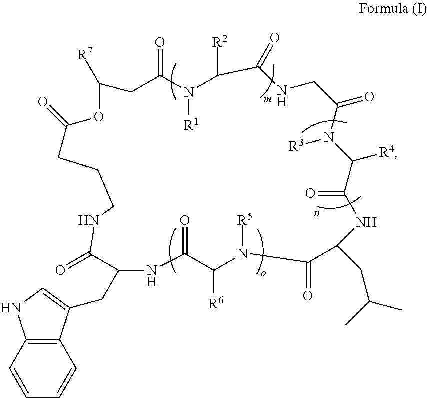

or a racemate, an enantiomer, a diastereomer, a pharmaceutically acceptable salt, or a derivative thereof, or

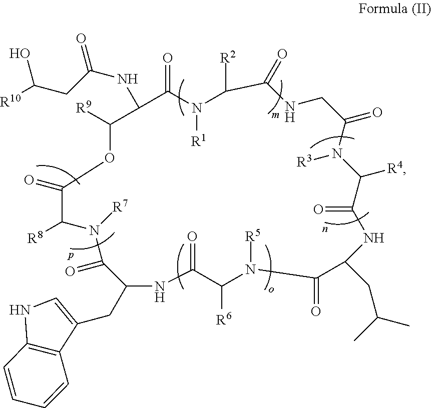

or a racemate, an enantiomer, a diastereomer, a pharmaceutically acceptable salt, or a derivative thereof.

In some embodiments, each occurrence of R¹, R², R³, R⁴, R⁵, R⁶, R⁷, R⁸, R⁹, and R¹⁰ is independently selected from hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In some embodiments, R¹ and R² are optionally fused or joined to form a ring.

In some embodiments, R³ and R⁴ are optionally fused or joined to form a ring.

In some embodiments, R⁵ and R⁶ are optionally fused or joined to form a ring.

In some embodiments, R⁷ and R⁸ are optionally fused or joined to form a ring.

In some embodiments, each occurrence of m, n, o, and p is independently an integer from 0 to 100.

In some embodiments, the compound having the structure of Formula (I) is a compound having the structure of Formula (Ia)

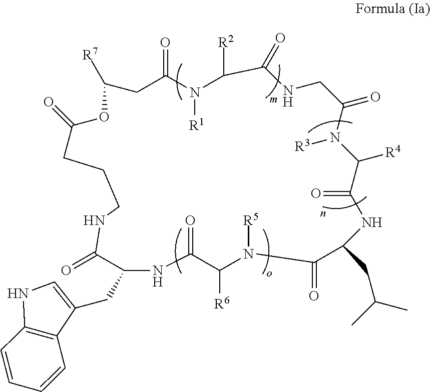

or a racemate, an enantiomer, a diastereomer, a pharmaceutically acceptable salt, or a derivative thereof.

In some embodiments, the compound having the structure of Formula (II) is a compound having the structure of Formula (IIa)

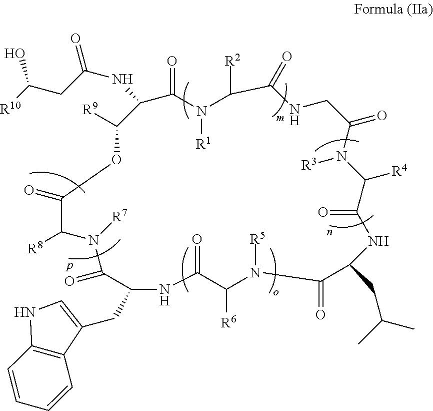

or a racemate, an enantiomer, a diastereomer, a pharmaceutically acceptable salt, or a derivative thereof.

In some embodiments, each occurrence of R¹, R², R³, R⁴, R⁵, R⁶, R⁷, R⁸, R⁹, and R¹⁰ is independently selected from hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof.

In some embodiments, R¹ and R² are optionally fused or joined to form a ring.

In some embodiments, R³ and R⁴ are optionally fused or joined to form a ring.

In some embodiments, R⁵ and R⁶ are optionally fused or joined to form a ring.

In some embodiments, each occurrence of m, n, and o is independently an integer from 0 to 100.

In some embodiments, the compound is a compound selected from

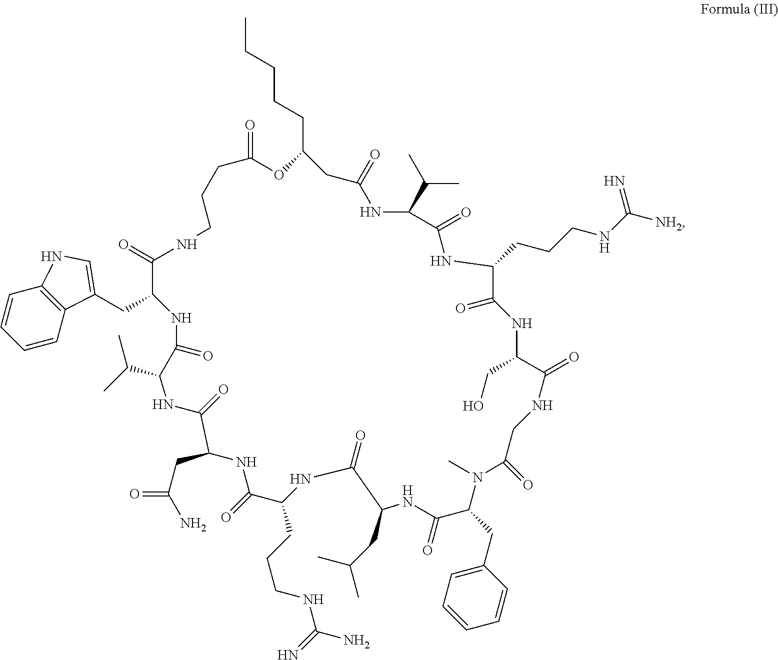 [SMILES: CCCCC[C@@H]1CC(=O)N[C@@H](C(C)C)C(=O)N[C@H](CCCNC(=N)N)C(=O)N[C@@H](CO)C(=O)NCC(=O)N(C)[C@H](Cc2ccccc2)C(=O)N[C@@H](CC(C)C)C(=O)N[C@H](CCCNC(=N)N)C(=O)N[C@@H](CC(N)=O)C(=O)N[C@H](C(C)C)C(=O)N[C@H](Cc2c[nH]c3ccccc23)C(=O)NCCCC(=O)O1]

or a racemate, an enantiomer, a diastereomer, a pharmaceutically acceptable salt, or a derivative thereof,

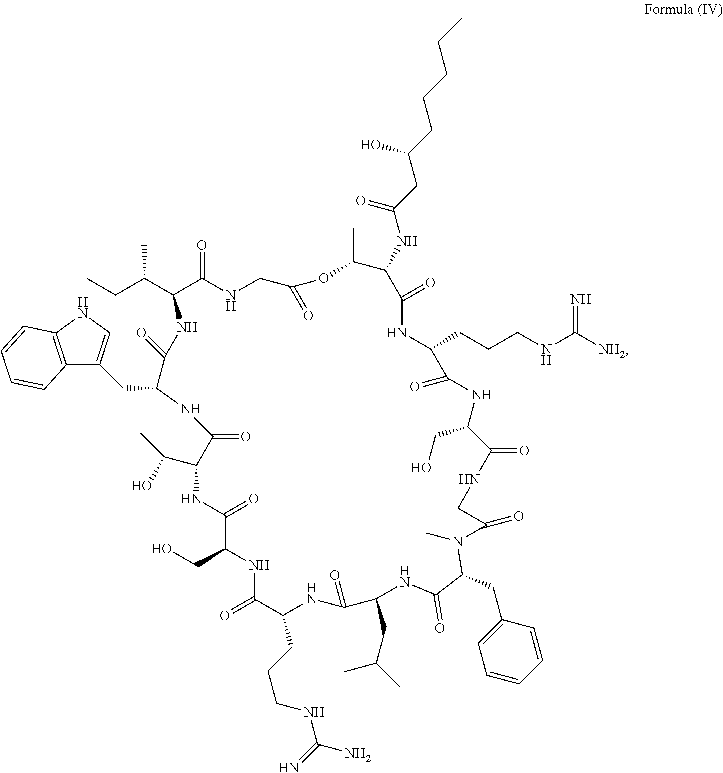 [SMILES: CCCCC[C@@H](O)CC(=O)N[C@@H]1C(=O)N[C@H](CCCNC(=N)N)C(=O)N[C@@H](CO)C(=O)NCC(=O)N(C)[C@H](Cc2ccccc2)C(=O)N[C@@H](CC(C)C)C(=O)N[C@H](CCCNC(=N)N)C(=O)N[C@@H](CO)C(=O)N[C@H]([C@@H](C)O)C(=O)N[C@H](Cc2c[nH]c3ccccc23)C(=O)N[C@@H]([C@@H](C)CC)C(=O)NCC(=O)O[C@@H]1C]

or a racemate, an enantiomer, a diastereomer, a pharmaceutically acceptable salt, or a derivative thereof,

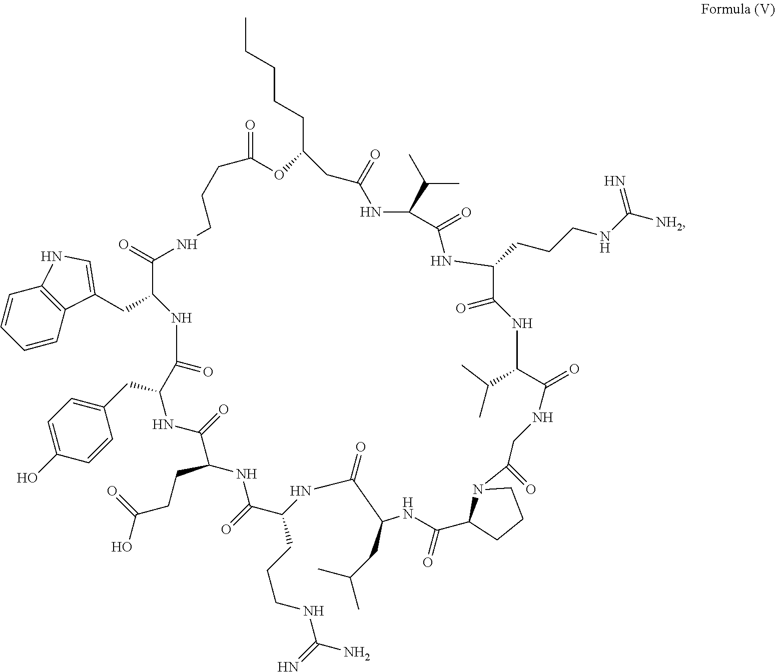 [SMILES: CCCCC[C@@H]1CC(=O)N[C@@H](C(C)C)C(=O)N[C@H](CCCNC(=N)N)C(=O)N[C@@H](C(C)C)C(=O)NCC(=O)N2CCC[C@H]2C(=O)N[C@@H](CC(C)C)C(=O)N[C@H](CCCNC(=N)N)C(=O)N[C@@H](CCC(=O)O)C(=O)N[C@H](Cc2ccc(O)cc2)C(=O)N[C@H](Cc2c[nH]c3ccccc23)C(=O)NCCCC(=O)O1]

or a racemate, an enantiomer, a diastereomer, a pharmaceutically acceptable salt, or a derivative thereof,

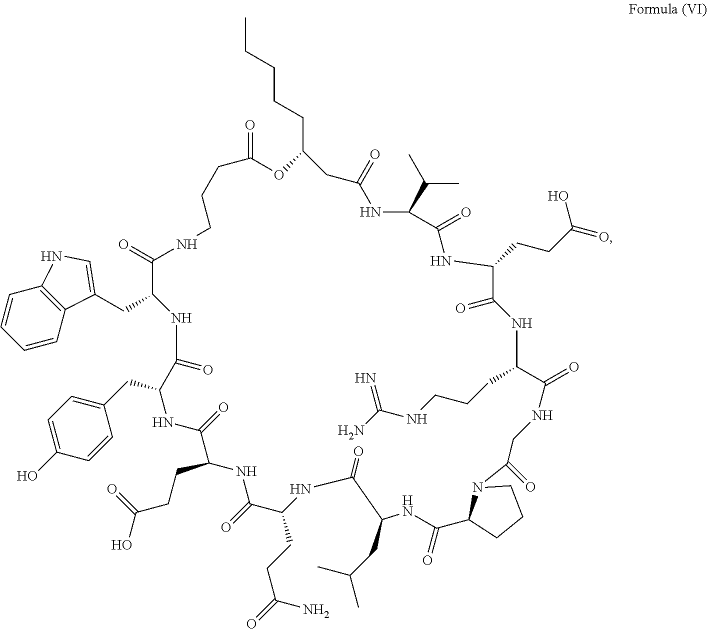 [SMILES: CCCCC[C@@H]1CC(=O)N[C@@H](C(C)C)C(=O)N[C@H](CCC(=O)O)C(=O)N[C@@H](CCCNC(=N)N)C(=O)NCC(=O)N2CCC[C@H]2C(=O)N[C@@H](CC(C)C)C(=O)N[C@H](CCC(N)=O)C(=O)N[C@@H](CCC(=O)O)C(=O)N[C@H](Cc2ccc(O)cc2)C(=O)N[C@H](Cc2c[nH]c3ccccc23)C(=O)NCCCC(=O)O1]

or a racemate, an enantiomer, a diastereomer, a pharmaceutically acceptable salt, or a derivative thereof,

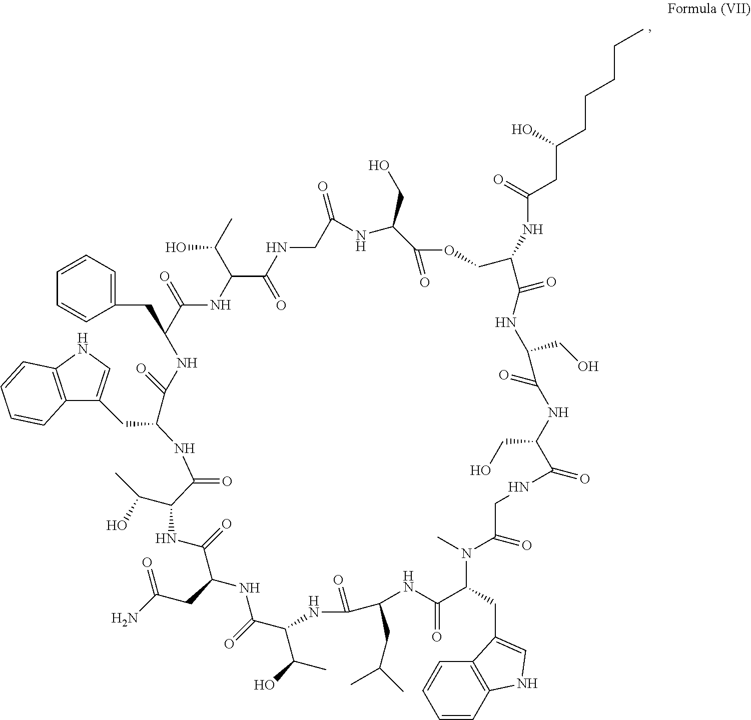

or a racemate, an enantiomer, a diastereomer, a pharmaceutically acceptable salt, or a derivative thereof, or

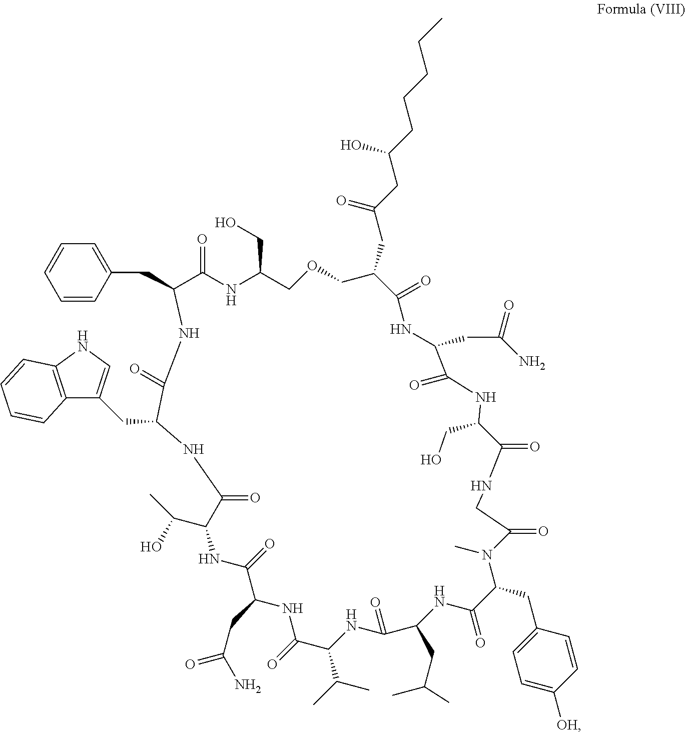

or a racemate, an enantiomer, a diastereomer, a pharmaceutically acceptable salt, or a derivative thereof.

In some embodiments, the compound specifically binds to menaquinone.

In one aspect, the present invention also provides a pharmaceutical composition comprising at least one compound of the present invention.

In another aspect, the present invention provides an isolated nucleic acid encoding at least one compound of the present invention or a racemate, an enantiomer, a diastereomer, a pharmaceutically acceptable salt, or a derivative thereof.

In another aspect, the present invention provides a genetically engineered cell comprising at least one compound of the present invention or a racemate, an enantiomer, a diastereomer, a pharmaceutically acceptable salt, or a derivative thereof.

In another aspect, the present invention provides a genetically engineered cell encoding at least one compound of the present invention or a racemate, an enantiomer, a diastereomer, a pharmaceutically acceptable salt, or a derivative thereof.

In one aspect, the present invention also provides a method of treating or preventing a bacterial infection in a subject in need thereof. In some embodiments, the method comprises administering at least one compound of the present invention or a composition thereof to the subject.

In some embodiments, the subject is exposed to or infected with a pathogen. In some embodiments, the pathogen is bacteria. In some embodiments, the bacteria is selected from drug resistant bacteria, gram positive bacteria, and any combination thereof.

In some embodiments, the method further comprises administering a second therapeutic. In some embodiments, the second therapeutic is an antibiotic.

In one aspect, the present invention also provides a method of inhibiting the growth of or killing a bacterial cell. In some embodiments, the method comprises contacting the bacterial cell with at least one compound of the present invention or a composition thereof.

In one aspect, the present invention also provides a method of biosynthesizing a compound comprising the amino acid sequence (X^{A})ₐG(X^{B})_{b}L(X^{C})_{c}W(X^{D})_{d}.

In some embodiments, the method comprises a) providing a nucleic acid to a host, wherein the nucleic acid encodes the amino acid sequence (X^{A})ₐG(X^{B})_{b}L(X^{C})_{c}W(X^{D})_{d} or a fragment thereof, b) incubating the host in a growth medium; and c) isolating the compound from the host or the growth medium.

In some embodiments, each occurrence of X^{A}, X^{B}, X^{C}, and X^{D} is independently selected from a natural amino acid, functionalized natural amino acid, unnatural amino acid, functionalized unnatural amino acid, or any combination thereof.

In some embodiments, each occurrence of a, b, c, and d is independently an integer from 0 to 100.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts a schematic representation of identification of BGCs predicted to encode new MBAs. Using a sequence based soil metagenome BGC discovery pipeline, three BGCs were identified that show high A-domain sequence identity and similar overall gene organization to known MBA BGCs. Each of these encoded a new MBA. Using the conserved GXLXXXW motif that were detected in structurally diverse MBAs to search an in-house database of predicted NRP (p-NRP) structures three additional BGCs were identified that would encode new MBAs. The GXLXXXW motif that was found in all MBAs is predicted to represent the minimal MK binding motif that is necessary for the antibacterial activity of this underexplored and structurally diverse class of natural antibiotics.

FIG. 2 depicts a schematic representation of synthesis and antibacterial activity of syn-BNPs based on BGCs predicted to encode MBAs. The (R)-3-hydroxy-octanoic acid derivatized linear peptides that are predicted to be encoded by MBA BGCs were cyclized through either the hydroxyl group of the fatty acid (cFA) or through a nucleophilic amino acid side-chain (cSC). When the first amino acid was predicted to contain a nucleophilic side chain (i.e., a serine or threonine) both the cFA and cSC analogs were synthesized (MBA2, MBA5 and MBA6). In the case of MBA5-cSC2, the serine at position 2 was also used for cyclization. If the first amino acid of peptide did not contain a nucleophilic side chain, only a cFA derivative was produced (MBA1, MBA3 and MBA4). syn-BNPs marked with an asterisk were the most active to arise from each BGC and assumed to be the “naturally cyclized” versions of the potential MBA. All MIC assays were done in duplicate (n=2).

FIG. 3 depicts representative results demonstrating bactericidal effects and mode-of-action analysis of six new MBAs. a, Bactericidal activity of MBA1-6 against S. aureus USA300. Cultures were incubated with each antibiotic at 2× its MIC. The number of viable cells was counted (n=3). Cultures were plated at defined times points to determine CFU per mL. b, Effect of MBA1-6 on S. aureus membrane lysis was determined using SYTOX fluorescence assay. c, The S. aureus antibacterial activity of MBA1-6 was determined in the presence of different concentrations of menaquinone (MK) (blue) or ubiquinone (UQ) (orange) (n=2). d, The MICs (g/mL) of MBA1-6 against S. aureus mutants deficient in MK biosynthesis (ΔmenA or ΔmenB) (n=2). Lysocin and Van (vancomycin) were included as positive (MK binding) and negative (non-MK binding) antibiotic controls.

FIG. 4 depicts representative results demonstrating anti-Mtb activity and mode of action analysis of new MBAs. a, MIC (g/mL) values of all known and new MBAs against a panel of Mtb strains, including two BSL2 strains (mc2 6206 and mc2 7901), a wild-type strain (H37Rv) and four multidrug-resistant (MDR) clinical isolates (n=2). The four MDR strains (800, 4557, 10571 and 116) are resistant to rifampicin, rifampicin, ethambutol/isoniazid/rifampicin/streptomycin and ethambutol/isoniazid/para-aminosalicylic acid, respectively. b, Effects of MK (blue) or UQ (orange) on the antibacterial activity of MBA1-3 against Mtb mc2 6206 (n=2). c, The ability of Mtb-active MBAs to permeabilize the Mtb membrane was examined using a 3′-dipropylthiadicarboncyanine iodide [DiSC3(5)]fluorescence assay. Verapamil and rifampicin were used as positive and negative depolarization controls, respectively. d, Activity of the three Mtb-active MBAs against Mtb mc2 6206/mLux itself (MIC, n=2) and Mtb mc2 6206/mLux in a macrophage infection assay (IC₅₀, n=3).

FIG. 5 depicts representation structures of six new MBAs grouped by structural family. a, Phylogenetic tree of linear MBA peptide sequences. The branches on the tree are labeled with the name of the MBA and the source of its BGC. In this study congeners of two known MBAs (b) as well as two new MBA structural families (c, d) were identified. All MBAs share the conserved GXLXXXW motif (blue) that is predicted to be the minimal sequence that is associated with MK-binding as a mode of action. The conserved residues within each MBA family are highlighted. In accordance with the long standing tradition of giving bioactive natural products trivial names, wameb (MBA1, WBP-29479A1-like menaquinone-binding antibiotic), lysomeb (MBA2. lysocin E-like menaquinone-binding antibiotic), metameb (MBA3, metagenome menaquinone-binding antibiotic), alcameb (MBA4, P. alcaliphilus menaquinone-binding antibiotic), tabameb (MBA5, D. tabacisoli menaquinone-binding antibiotic) and mobimeb (MBA6, D. mobilis menaquinone-binding antibiotic).

FIG. 6 depicts representative results demonstrating MBA3 (a) and MBA6 (b) are effective against S. aureus infections in mice. Either MBA3 or MBA6 was subcutaneously injected 1 h after intraperitoneal administration of S. aureus COL into mice (n=5). 30% solutol was used as the vehicle.

FIG. 7 depicts representative twenty-two predicted peptides contain complete or partial proposed minimal MK-binding motifs. The predicted peptide from D. mobilis (green) contains the entire “GXLXXXW” motif. The predicted peptides from D. tabacisoli and four different Paracoccus strains (red) contain the “GXL” portion of the motif.

FIG. 8 depicts representative results demonstrating spectrum of activity for MBAs 1 through 6 (MICs in g/mL). The highest concentration tested was 64 μg/mL for all microbes except for M. tuberculosis H37Rv, which was tested at 20 μg/mL. The highest concentration tested for HEK293 human cells was 32 μg/mL.

FIG. 9 depicts representative results demonstrating antibiotic activity of MBAs 1 through 6 and four known lipid II binding antibiotics against S. aureus mutants (tn:hemB or ΔmenA) as well as Enterococcus and Streptococcus strains that either produce or do not produce MK. MIC in g/mL, highest concentration tested was 64 μg/mL.

FIG. 10 depicts representative results demonstrating antibiotic activity of MBAs 1 through 6 against E. coli BAS849 grown under aerobic and anaerobic conditions. E. coli DH5a was used as the control. MIC in g/mL, highest concentration tested was 64 μg/mL.

FIG. 11 depicts representative comprehensive overview of all mutated genes in each MBA resistant S. aureus strain

FIG. 12 depicts representative phylogenetic analysis of eSNaPD hits from six conserved A-domains found in the BGCs of the three known MBAs. Phylogenetic analysis of eSNaPD hits (a) and predicted peptide sequences of recovered clones (b). All the hits at an e-value ≤10-45 from A-domain analysis of L-Leu-6 encoded new MBAs and formed a separate, well-defined clade with A-domains of three known MBAs, which indicated that L-Leu-6 in the proposed minimal MK-binding motif were encoded by the most highly conserved A domain in MBA-family peptides.

FIG. 13 depicts representative results demonstrating three potential MBA BGCs from eSNaPD-guided soil metagenomic mining. Comparison of NRPS gene organization (a) as well as amino acid substrates (b) between the three known MBA BGCs and the three potential MBA BGCs were cloned from soil metagenomes.

FIG. 14 depicts representative flowchart of the process used to create the predicted non-ribosomal peptide (p-NRP) database.

FIG. 15 depicts representative results demonstrating predicted MBA peptide sequences identified in a motif search of the p-NRP database (a) and the BGCs associated with these predicted peptides (b).

FIG. 16 depicts representative results demonstrating predicted monomer building blocks used by the six potential MBA BGCs that were identified. Three A-domain substrate prediction sources, including NRPSPredictor2, Stachelhaus and the in-house manual examination of characterized BGCs, were used to predict the substrate of each A-domain. The Stachelhaus code that consists of 10 A-domain active site residues (positions 235, 236, 239, 278, 299, 301, 322, 330, 331 and 517) are shown for each A-domain. The asterisk represents although MBA3 gene cluster is incomplete due to the lack of AD 11 and thioesterase domain, the loading building block of AD 11 in MBA3 could be proposed to be GABA based on the high similarities of MBA3 peptide sequence and AD 11 associated condensation domain to those of WBP-29479A1.

FIG. 17 depicts representative results demonstrating the structures (a) and anti-bacterial activities (b) of the N-acylated peptides associated with known MBAs cyclized in two different ways. The (R)-3-hydroxy-octanoic acid analogs of lysocin E, WBP-29479A1 and the deoxy version of WAP-8294A1 shown here were synthesized in this study. B. subtilis 168 1A1, S. aureus USA300, S. epidemidis RP62A and M. tuberculosis H37Rv were used as tested strains.

FIG. 18 depicts representative results demonstrating membrane depolarization activity and resistance frequency of MBAs 1 through 6. a. The effect of each MBA on S. aureus membrane potential was measured using 3,3′-Dipropylthiadicarbocyanine iodide [DiSC3(5)]. Vancomycin (Van) and lysocin were used as the negative and positive controls, respectively. b. Resistance frequency of MBAs 1 through 6 against S. aureus USA300 in the presence of 4× the MIC of each antibiotic.

FIG. 19 depicts representative results demonstrating isothermal titration of 1:1 (mol/mol) DOPC:DOPG vesicles containing MK into each MBA.

FIG. 20 depicts representative results demonstrating isothermal titration of 1:1 (mol/mol) DOPC:DOPG vesicles containing UQ into each MBA.

FIG. 21 depicts representative results demonstrating correlation between antibiotic activity and MK binding affinity for active or inactive syn-BNP MBAs. a. Isothermal titration of 1:1 (mol/mol) DOPC:DOPG vesicles containing MK into the four additional syn-BNPs generated in FIG. 2. b. Comparison of Kd values and MICs against S. aureus USA300 for all syn-BNP MBAs in FIG. 2.

FIG. 22 depicts representative results demonstrating isothermal titration of DOPC vesicles containing lipid II into active or inactive analogs of lysocin, MBA3 and MBA6. Each pair of active and inactive compounds showed no lipid II binding or similar low lipid II binding affinities, indicating that these are non-specific interactions. Nisin was used as the positive control.

FIG. 23 depicts representative results demonstrating MK was undetectable in the tn:hemB mutant. a. MK was extracted from cultures of both S. aureus Newman and USA300 (MK positive controls), a menA deletion mutant (MK negative control) and a hemB transposon insertion mutant (tn:hemB). Each extract was resolved by thin layer chromatography (TLC) and visualized using 254 nm lamp. Commercially available menaquinone-4 (MK4) was included as a reference. b. The MK4 standard and MK extracts from all four S. aureus strains were analyzed by high-resolution mass spectrometry (HRMS) in positive ion mode (ES+). The observed m/z values were based on the addition of a single proton [MH]+.

FIG. 24 depicts representative results demonstrating antibiotic activity and MK binding of MBA3 with single point mutations in the proposed minimal MK-binding motif. MIC in g/mL, highest concentration tested was 64 μg/mL.

FIG. 25 depicts representative sources of MBA BGCs. Summary of sources for all known and new MBA BGCs. The numbers represent the MBA BGCs that are identified or predicted from different bacterial species or metagenomes.

FIG. 26 depicts representative results demonstrating high-resolution mass spectrometry (HRMS) (ES+) data for all compounds synthesized in this study. The calculated and observed m/z values are based on the addition of a single proton [MH]+. All observed m/z values are within +5 ppm of the theoretical values.

FIG. 27 depicts representative results demonstrating HRMS MS/MS fragmentation data for MBA1. a. MS/MS spectrum of [M+2H]2+ at m/z 729.4302 of MBA1. b. Characteristic b- and y-series peptide fragmentation ions were assigned as shown.

FIG. 28 depicts representative results demonstrating HRMS MS/MS fragmentation data for MBA2. a. MS/MS spectrum of [M+2H]2+ at m/z 760.4229 of MBA2. b. Characteristic b- and y-series peptide fragmentation ions were assigned as shown.

FIG. 29 depicts representative results demonstrating HRMS MS/MS fragmentation data for MBA3. a. MS/MS spectrum of [M+2H]2+ at m/z 742.9261 of MBA3. b. Characteristic b- and y-series peptide fragmentation ions were assigned as shown.

FIG. 30 depicts representative results demonstrating HRMS MS/MS fragmentation data for MBA4. a. MS/MS spectrum of [M+2H]2+ at m/z 743.3888 of MBA4. b. Characteristic b- and y-series peptide fragmentation ions were assigned as shown.

FIG. 31 depicts representative results demonstrating HRMS MS/MS fragmentation data for MBA5. a. MS/MS spectrum of [M+2H]2+ at m/z 835.3961 of MBA5. b. Characteristic b- and y-series peptide fragmentation ions were assigned as shown.

FIG. 32 depicts representative results demonstrating HRMS MS/MS fragmentation data for MBA6. a. MS/MS spectrum of [M+2H]2+ at m/z 756.8684 of MBA6. b. Characteristic b- and y-series peptide fragmentation ions were assigned as shown.

FIG. 33 depicts representative results demonstrating ¹H and ¹³C NMR spectra of MBA1 in DMSO-d6.

FIG. 34 depicts representative results demonstrating ¹H and ¹³C NMR spectra of MBA2 in DMSO-d6.

FIG. 35 depicts representative results demonstrating ¹H and ¹³C NMR spectra of MBA3 in DMSO-d6.

FIG. 36 depicts representative results demonstrating ¹H and ¹³C NMR spectra of MBA4 in DMSO-d6.

FIG. 37 depicts representative results demonstrating ¹H and ¹³C NMR spectra of MBA5 in DMSO-d6.

FIG. 38 depicts representative results demonstrating ¹H and ¹³C NMR spectra of MBA6 in DMSO-d6.

### DETAILED DESCRIPTION

The present invention is based, in part, on the unexpected discovery of menaquinone-binding compounds as antibiotics which have activity against multidrug resistant pathogens. In one embodiment, the present invention provides compounds or a therapeutic compound comprising a desired activity. In one embodiment, the compound is an antibiotic. In one embodiment, the antibiotic compound of the invention can be used in the treatment of bacterial infections. In one embodiment, the antibiotic compound of the invention can be used in the treatment of gram positive bacterial infections. In certain embodiments, the use of the antibiotic compound of the invention in the treatment of bacterial infections optionally includes a pharmaceutically acceptable carrier, excipient or adjuvant.

In one embodiment, the compound can be biosynthesized via heterologous expression of a biosynthetic gene. Thus, in one aspect, the invention provides compounds and methods for synthesizing menaquinone-binding compounds. In one embodiment, the invention provides a nucleic acid encoding menaquinone-binding compounds. In one embodiment, the nucleic acid is an isolated nucleic acid. In one embodiment, the nucleic acid is transformed into a cell.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, +1%, or ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

To “treat” a disease as the term is used herein, means to reduce the frequency or severity of at least one sign or symptom of a disease or disorder experienced by a subject.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term, “biologically active” or “bioactive” can mean, but is in no way limited to, the ability of an agent or compound to effectuate a physiological change or response. The response may be detected, for example, at the cellular level, for example, as a change in growth and/or viability, gene expression, protein quantity, protein modification, protein activity, or combination thereof; at the tissue level; at the systemic level; or at the organism level. For example, as used herein, biologically active molecules include but are not limited to any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes, small molecule drugs, dyes, lipids, nucleosides, oligonucleotides, cells, viruses, liposomes, microparticles and micelles. Classes of biologically active agents that are suitable for use with the invention include, but are not limited to, antibiotics, fungicides, anti-viral agents, anti-inflammatory agents, anti-tumor agents, cardiovascular agents, anti-anxiety agents, hormones, growth factors, steroidal agents, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “amino acid”, “amino acidic monomer”, or “amino acid residue” refer to any of the twenty naturally occurring amino acids including synthetic amino acids with unnatural side chains and including both D and L optical isomers.

In the context of the invention, term “natural amino acid” means any amino acid which is found naturally in vivo in a living being. Natural amino acids therefore include amino acids coded by mRNA incorporated into proteins during translation but also other amino acids found naturally in vivo which are a product or by-product of a metabolic process, such as for example ornithine which is generated by the urea production process by arginase from L-arginine. In the invention, the amino acids used can therefore be natural or not. Namely, natural amino acids generally have the L configuration but also, according to the invention, an amino acid can have the L or D configuration.

A “non-naturally encoded amino acid” refers to an amino acid that is not one of the 20 common amino acids or pyrolysine or selenocysteine. The term “non-naturally encoded amino acid” includes, but is not limited to, amino acids that occur naturally by modification of a naturally encoded amino acid (including but not limited to, the 20 common amino acids or pyrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. Furthermore, peptides of the invention may include amino acid mimentics, and analogs. Recombinant forms of the peptides can be produced according to standard methods and protocols which are well known to those of skill in the art, including for example, expression of recombinant proteins in prokaryotic and/or eukaryotic cells followed by one or more isolation and purification steps, and/or chemically synthesizing peptides or portions thereof using a peptide sythesizer.

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

Non-limiting examples of agents suitable for formulation with the, e.g., compounds provided by the instant invention include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, “derivatives” are compositions formed from the native compounds either directly, by modification, or by partial substitution. As used herein, “analogs” are compositions that have a structure similar to, but not identical to, the native compound.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

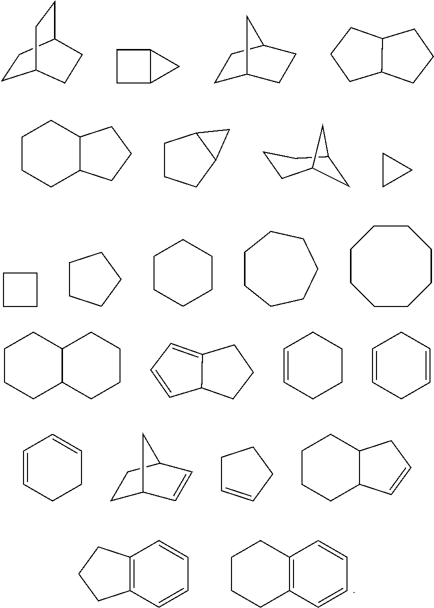

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, Si, P, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

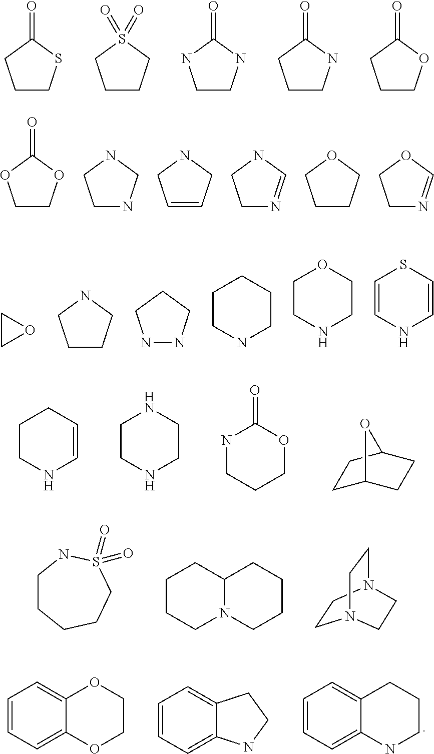

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₄)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₄)alkyl” means an aryl-(C₁-C₄)alkyl functional group in which the aryl group is substituted. Preferred is substituted aryl(CH₂)—. Similarly, the term “heteroaryl-(C₁-C₄)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₄)alkyl” means a heteroaryl-(C₁-C₄)alkyl functional group in which the heteroaryl group is substituted. Preferred is substituted heteroaryl-(CH₂)—.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₄)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected discovery of menaquinone-binding compounds as antibiotics which have activity against multidrug resistant pathogens. In one embodiment, the present invention provides compounds or a therapeutic compound comprising a desired activity. In one embodiment, the compound is an antibiotic. In one embodiment, the antibiotic compound of the invention can be used in the treatment of bacterial infections. In one embodiment, the antibiotic compound of the invention can be used in the treatment of gram positive bacterial infections. In certain embodiments, the use of the antibiotic compound of the invention in the treatment of bacterial infections optionally includes a pharmaceutically acceptable carrier, excipient or adjuvant.

In one embodiment, the compound can be biosynthesized via heterologous expression of a biosynthetic gene. Thus, in one aspect, the invention provides compounds and methods for synthesizing menaquinone-binding compounds. In one embodiment, the invention provides a nucleic acid encoding menaquinone-binding compounds. In one embodiment, the nucleic acid is an isolated nucleic acid. In one embodiment, the nucleic acid is transformed into a cell.

### Compounds

In one aspect, the present invention provides a compound or a racemate, an enantiomer, a diastereomer thereof, a pharmaceutically acceptable salt, or a derivative thereof comprising the amino acid sequence (X^{A})ₐG(X^{B})_{b}L(X^{C})_{c}W(X^{D})_{d}. In one aspect of the invention, the compound is a cyclic compound. In one aspect of the invention, the compound specifically binds to menaquinone.

In some embodiments, each occurrence of X^{A}, X^{B}, X^{C}, and X^{D} is independently selected from a natural amino acid, functionalized natural amino acid, unnatural amino acid, functionalized unnatural amino acid, or any combination thereof.

In some embodiments, each occurrence of a, b, c, and d is independently an integer from 0 to 100. In some embodiments, each occurrence of a, b, c, and d is independently an integer from 0 to 10.

For example, in one embodiment, a is an integer of 0. In one embodiment, a is an integer of 1. In one embodiment, a is an integer of 2. In one embodiment, a is an integer of 3. In one embodiment, a is an integer of 4. In one embodiment, a is an integer of 5. In one embodiment, a is an integer of 6. In one embodiment, a is an integer of 7. In one embodiment, a is an integer of 8. In one embodiment, a is an integer of 9. In one embodiment, a is an integer of 10.

In one embodiment, b is an integer of 0. In one embodiment, b is an integer of 1. In one embodiment, b is an integer of 2. In one embodiment, b is an integer of 3. In one embodiment, b is an integer of 4. In one embodiment, b is an integer of 5. In one embodiment, b is an integer of 6. In one embodiment, b is an integer of 7. In one embodiment, b is an integer of 8. In one embodiment, b is an integer of 9. In one embodiment, b is an integer of 10.

In one embodiment, c is an integer of 0. In one embodiment, c is an integer of 1. In one embodiment, c is an integer of 2. In one embodiment, c is an integer of 3. In one embodiment, c is an integer of 4. In one embodiment, c is an integer of 5. In one embodiment, c is an integer of 6. In one embodiment, c is an integer of 7. In one embodiment, c is an integer of 8. In one embodiment, c is an integer of 9. In one embodiment, c is an integer of 10.

In one embodiment, d is an integer of 0. In one embodiment, d is an integer of 1. In one embodiment, d is an integer of 2. In one embodiment, d is an integer of 3. In one embodiment, d is an integer of 4. In one embodiment, d is an integer of 5. In one embodiment, d is an integer of 6. In one embodiment, d is an integer of 7. In one embodiment, d is an integer of 8. In one embodiment, d is an integer of 9. In one embodiment, d is an integer of 10.

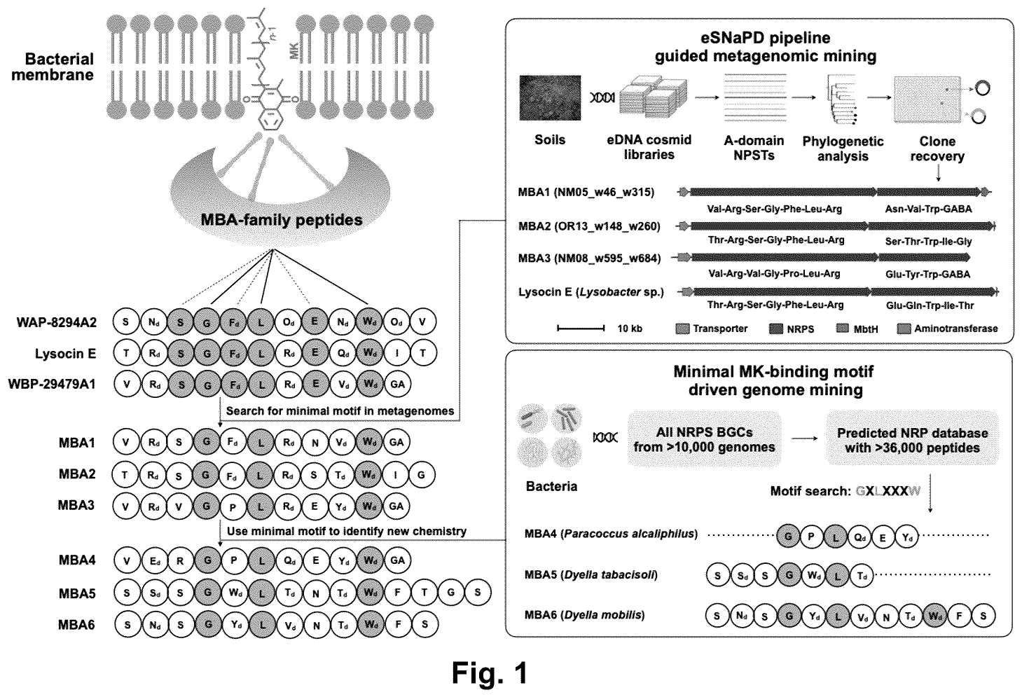

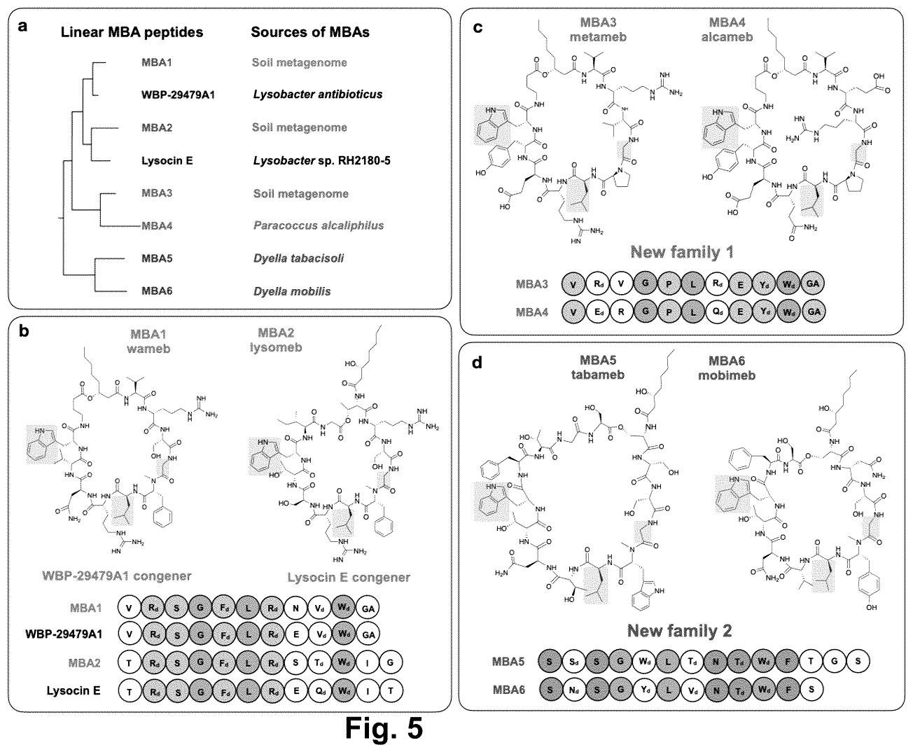

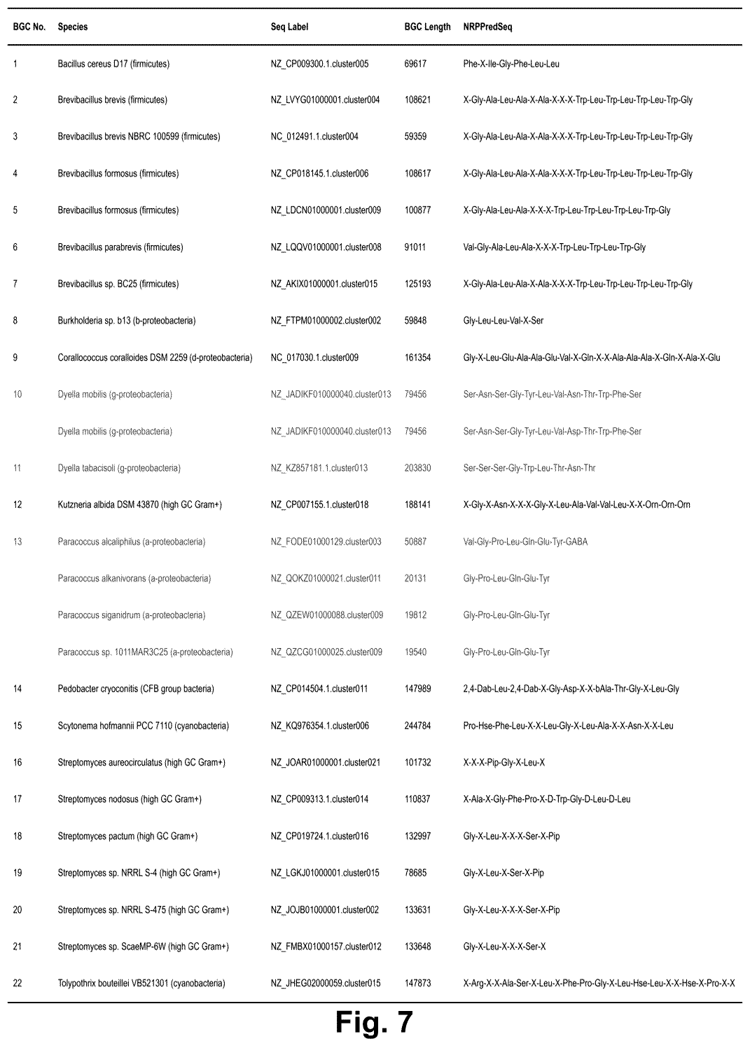

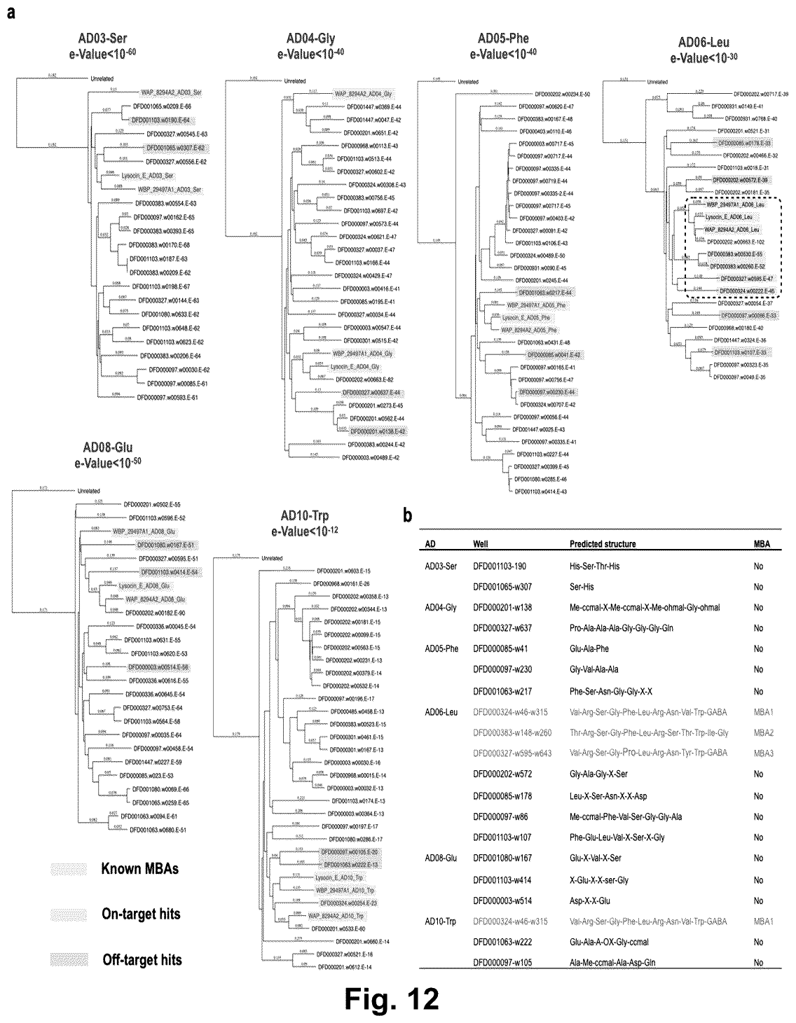

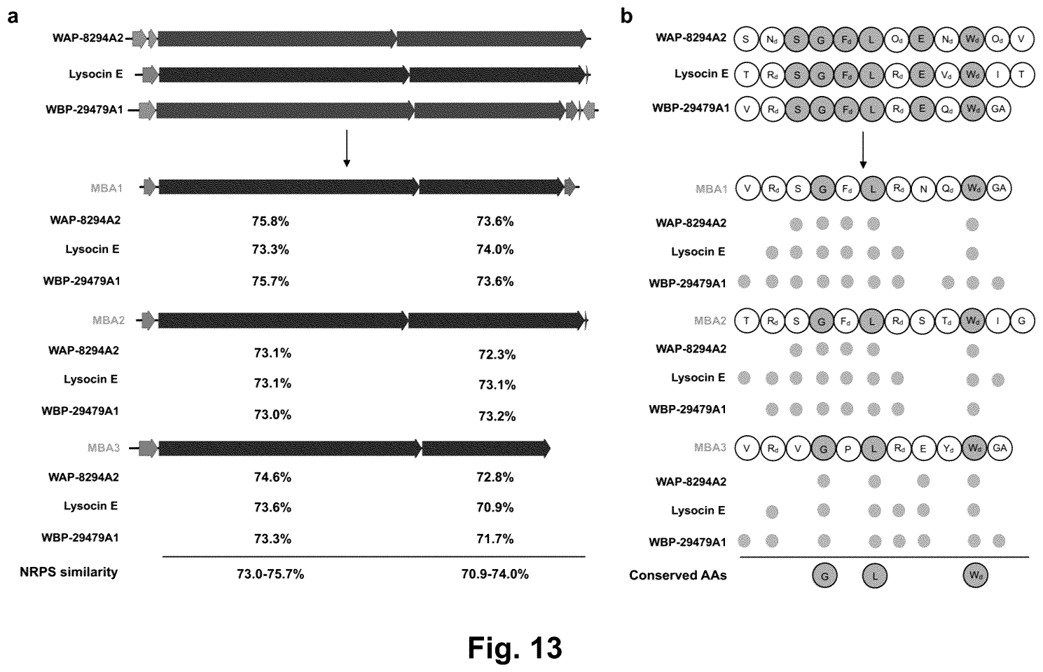

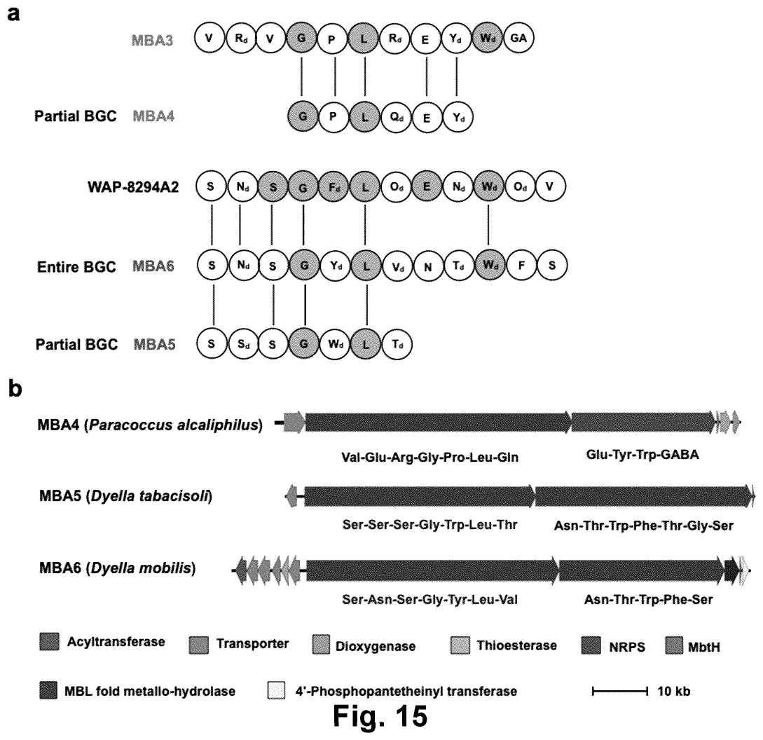

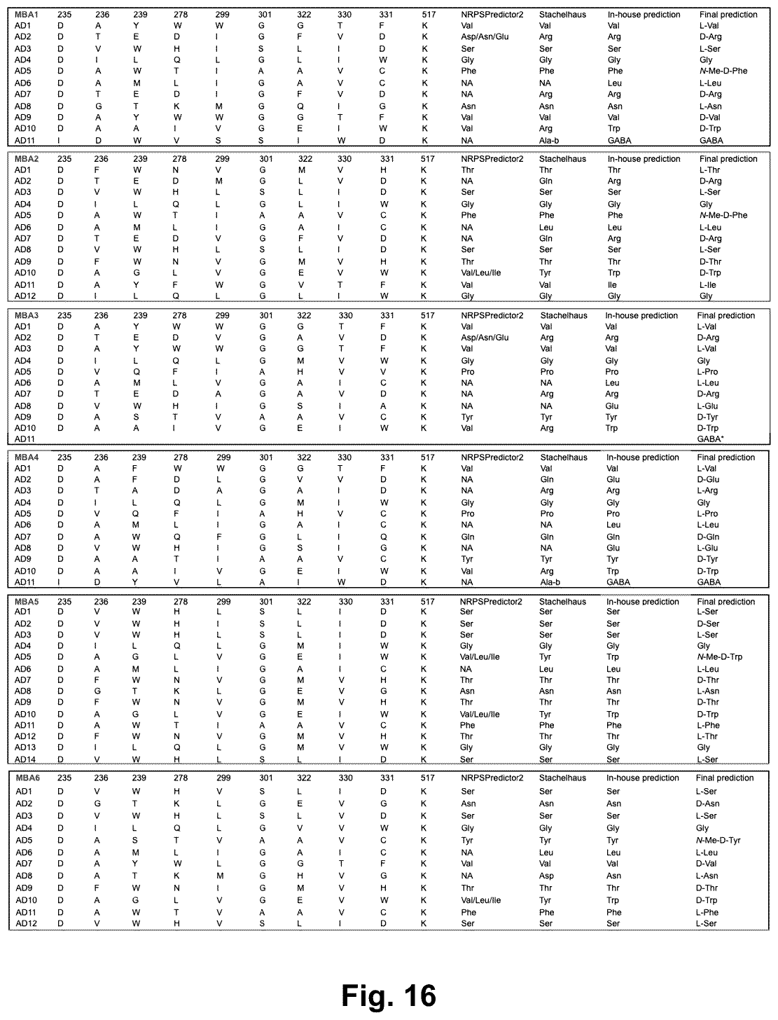

In some embodiments, the amino acid sequence (X^{A})ₐG(X^{B})_{b}L(X^{C})_{c}W(X^{D})_{d} comprises at least one amino acid sequence selected from at least one amino acid sequence, or a fragment thereof, selected from FIG. 1; at least one amino acid sequence, or a fragment thereof, selected FIG. 5; at least one amino acid sequence, or a fragment thereof, selected from FIG. 7; at least one amino acid sequence, or a fragment thereof, selected from FIG. 12; at least one amino acid sequence, or a fragment thereof, selected from FIG. 13; at least one amino acid sequence, or a fragment thereof, selected from FIG. 15; at least one amino acid sequence, or a fragment thereof, selected from FIG. 16; the amino acid sequence GXLXXXW; or any combination thereof.

For example, in one embodiment, the amino acid sequence (X^{A})ₐG(X^{B})_{b}L(X^{C})_{c}W(X^{D})_{d} comprises the amino acid sequence GXLXXXW. In some embodiments, each occurrence of X is independently selected from a natural amino acid, functionalized natural amino acid, unnatural amino acid, functionalized unnatural amino acid, or any combination thereof.

In one embodiment, the compound is a compound of general Formula (I)

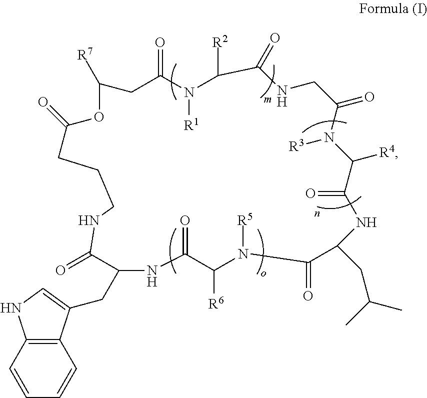

or a racemate, an enantiomer, a diastereomer, a pharmaceutically acceptable salt, or a derivative thereof.

In one embodiment, the compound is a compound of general Formula (II)

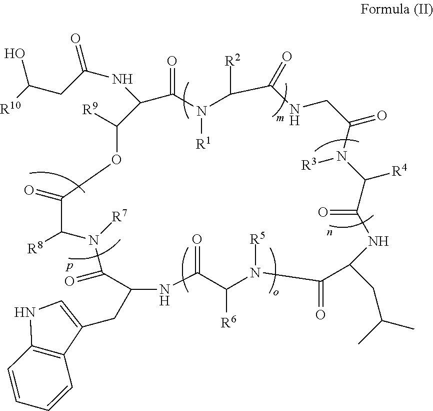

or a racemate, an enantiomer, a diastereomer, a pharmaceutically acceptable salt, or a derivative thereof.

In various embodiments, R¹ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R¹ is alkyl, alkenyl, aryl, aryl alkyl, aminoalkyl, hydroxyalkyl, or any combination thereof. In other embodiments, R¹ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In various embodiments, R² is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R² is alkyl, alkenyl, aryl, aryl alkyl, aminoalkyl, hydroxyalkyl, or any combination thereof. In other embodiments, R² is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In various embodiments, R³ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R³ is alkyl, alkenyl, aryl, aryl alkyl, aminoalkyl, hydroxyalkyl, or any combination thereof. In other embodiments, R³ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In various embodiments, R⁴ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R⁴ is alkyl, alkenyl, aryl, aryl alkyl, aminoalkyl, hydroxyalkyl, or any combination thereof. In other embodiments, R⁴ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In various embodiments, R⁵ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R⁵ is alkyl, alkenyl, aryl, aryl alkyl, aminoalkyl, hydroxyalkyl, or any combination thereof. In other embodiments, R⁵ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In various embodiments, R⁶ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R⁶ is alkyl, alkenyl, aryl, aryl alkyl, aminoalkyl, hydroxyalkyl, or any combination thereof. In other embodiments, R⁶ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In various embodiments, R⁷ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R⁷ is alkyl, alkenyl, aryl, aryl alkyl, aminoalkyl, hydroxyalkyl, or any combination thereof. In other embodiments, R⁷ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In various embodiments, R⁸ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R⁸ is alkyl, alkenyl, aryl, aryl alkyl, aminoalkyl, hydroxyalkyl, or any combination thereof. In other embodiments, R⁸ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In various embodiments, R⁹ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R⁹ is alkyl, alkenyl, aryl, aryl alkyl, aminoalkyl, hydroxyalkyl, or any combination thereof. In other embodiments, R⁹ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In various embodiments, R¹⁰ is hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combinations thereof. For example, in some embodiments, R¹⁰ is alkyl, alkenyl, aryl, aryl alkyl, aminoalkyl, hydroxyalkyl, or any combination thereof. In other embodiments, R¹⁰ is linear C₁-C₁₀ alkyl, branched C₁-C₁₀ alkyl, linear aryl-C₁-C₁₀ alkyl, branched aryl-C₁-C₁₀ alkyl, linear amino-C₁-C₁₀ alkyl, amino-branched C₁-C₁₀ alkyl, linear hydroxy-C₁-C₁₀ alkyl, hydroxy-branched C₁-C₁₀ alkyl, linear C₁-C₁₀ alkenyl, branched C₁-C₁₀ alkenyl, linear aryl-C₁-C₁₀ alkenyl, branched aryl-C₁-C₁₀ alkenyl, linear amino-C₁-C₁₀ alkenyl, amino-branched C₁-C₁₀ alkenyl, linear hydroxy-C₁-C₁₀ alkenyl, hydroxy-branched C₁-C₁₀ alkenyl, or any combination thereof.

In some embodiments, each occurrence of m, n, o, and p is independently an integer from 0 to 100. In some embodiments, each occurrence of m, n, o, and p is independently an integer from 0 to 10.

For example, in one embodiment, m is an integer of 0. In one embodiment, m is an integer of 1. In one embodiment, m is an integer of 2. In one embodiment, m is an integer of 3. In one embodiment, m is an integer of 4. In one embodiment, m is an integer of 5. In one embodiment, m is an integer of 6. In one embodiment, m is an integer of 7. In one embodiment, m is an integer of 8. In one embodiment, m is an integer of 9. In one embodiment, m is an integer of 10.

In one embodiment, n is an integer of 0. In one embodiment, n is an integer of 1. In one embodiment, n is an integer of 2. In one embodiment, n is an integer of 3. In one embodiment, n is an integer of 4. In one embodiment, n is an integer of 5. In one embodiment, n is an integer of 6. In one embodiment, n is an integer of 7. In one embodiment, n is an integer of 8. In one embodiment, n is an integer of 9. In one embodiment, n is an integer of 10.

In one embodiment, o is an integer of 0. In one embodiment, o is an integer of 1. In one embodiment, o is an integer of 2. In one embodiment, o is an integer of 3. In one embodiment, o is an integer of 4. In one embodiment, o is an integer of 5. In one embodiment, o is an integer of 6. In one embodiment, o is an integer of 7. In one embodiment, o is an integer of 8. In one embodiment, o is an integer of 9. In one embodiment, o is an integer of 10.

In one embodiment, p is an integer of 0. In one embodiment, p is an integer of 1. In one embodiment, p is an integer of 2. In one embodiment, p is an integer of 3. In one embodiment, p is an integer of 4. In one embodiment, p is an integer of 5. In one embodiment, p is an integer of 6. In one embodiment, p is an integer of 7. In one embodiment, p is an integer of 8. In one embodiment, p is an integer of 9. In one embodiment, p is an integer of 10.

For example, in some embodiments, each occurrence of R¹, R², R³, R⁴, R⁵, R⁶, R⁷, R⁸, R⁹, and R¹⁰ is independently selected from hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, or any combination thereof.

For example, in one embodiment, the compound of the present invention is a compound represented by Formula (III)

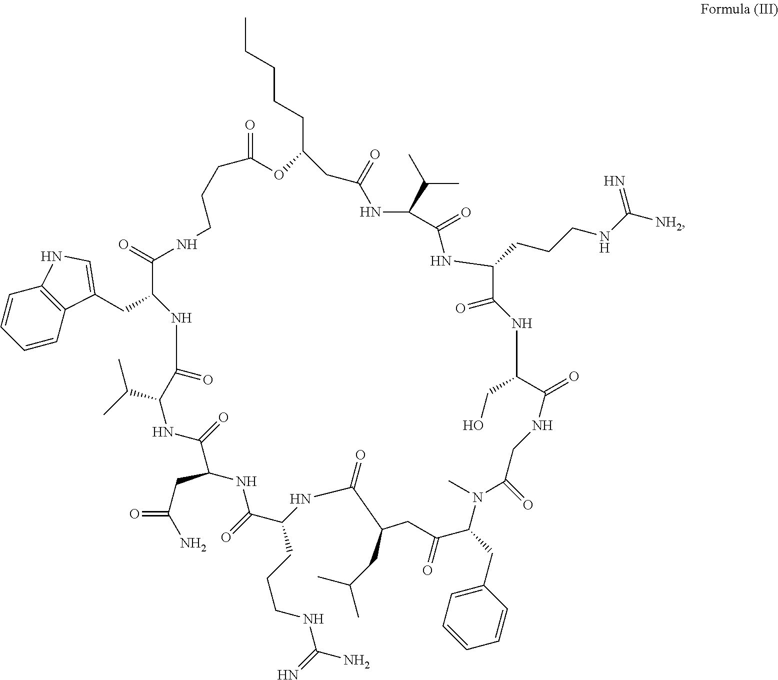 [SMILES: CCCCC[C@@H]1CC(=O)N[C@@H](C(C)C)C(=O)N[C@H](CCCNC(=N)N)C(=O)N[C@@H](CO)C(=O)NCC(=O)N(C)[C@H](Cc2ccccc2)C(=O)C[C@@H](CC(C)C)C(=O)N[C@H](CCCNC(=N)N)C(=O)N[C@@H](CC(N)=O)C(=O)N[C@H](C(C)C)C(=O)N[C@H](Cc2c[nH]c3ccccc23)C(=O)NCCCC(=O)O1]

or a racemate, an enantiomer, a diastereomer, a pharmaceutically acceptable salt, or a derivative thereof.

In one embodiment, the compound of the present invention is a compound represented by Formula (IV)

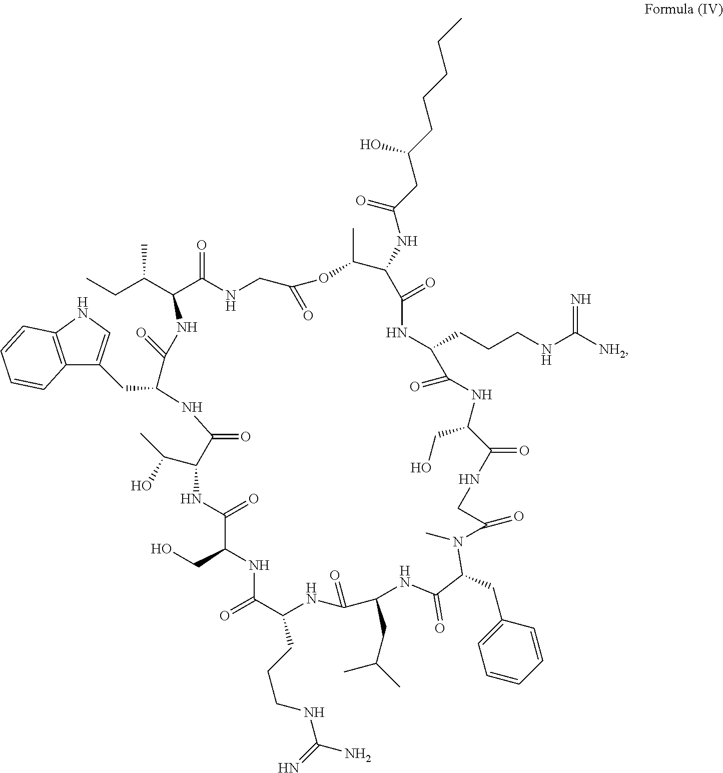 [SMILES: CCCCC[C@@H](O)CC(=O)N[C@@H]1C(=O)N[C@H](CCCNC(=N)N)C(=O)N[C@@H](CO)C(=O)NCC(=O)N(C)[C@H](Cc2ccccc2)C(=O)N[C@@H](CC(C)C)C(=O)N[C@H](CCCNC(=N)N)C(=O)N[C@@H](CO)C(=O)N[C@H]([C@@H](C)O)C(=O)N[C@H](Cc2c[nH]c3ccccc23)C(=O)N[C@@H]([C@@H](C)CC)C(=O)NCC(=O)O[C@@H]1C]

or a racemate, an enantiomer, a diastereomer, a pharmaceutically acceptable salt, or a derivative thereof.

In one embodiment, the compound of the present invention is a compound represented by Formula (V)

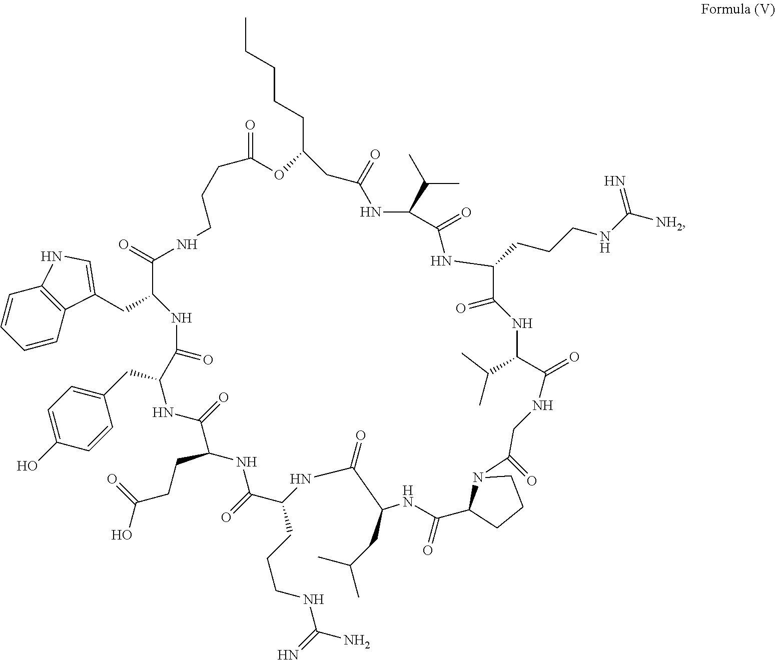 [SMILES: CCCCC[C@@H]1CC(=O)N[C@@H](C(C)C)C(=O)N[C@H](CCCNC(=N)N)C(=O)N[C@@H](C(C)C)C(=O)NCC(=O)N2CCC[C@H]2C(=O)N[C@@H](CC(C)C)C(=O)N[C@H](CCCNC(=N)N)C(=O)N[C@@H](CCC(=O)O)C(=O)N[C@H](Cc2ccc(O)cc2)C(=O)N[C@H](Cc2c[nH]c3ccccc23)C(=O)NCCCC(=O)O1]

or a racemate, an enantiomer, a diastereomer, a pharmaceutically acceptable salt, or a derivative thereof.

In one embodiment, the compound of the present invention is a compound represented by Formula (VI)

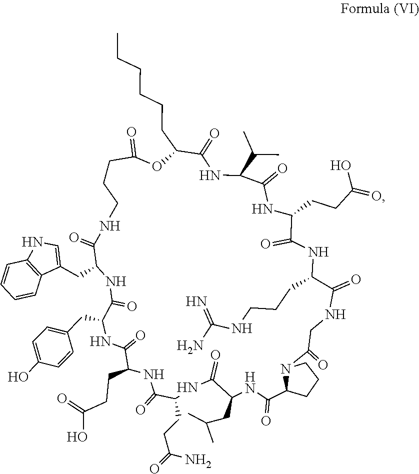 [SMILES: CCCCCC[C@H]1OC(=O)CCCNC(=O)[C@@H](Cc2c[nH]c3ccccc23)NC(=O)[C@@H](Cc2ccc(O)cc2)NC(=O)[C@H](CCC(=O)O)NC(=O)[C@@H](CCC(N)=O)NC(=O)[C@H](CC(C)C)NC(=O)[C@@H]2CCCN2C(=O)CNC(=O)[C@H](CCCNC(=N)N)NC(=O)[C@@H](CCC(=O)O)NC(=O)[C@H](C(C)C)NC1=O]

or a racemate, an enantiomer, a diastereomer, a pharmaceutically acceptable salt, or a derivative thereof.

In one embodiment, the compound of the present invention is a compound represented by Formula (VII)

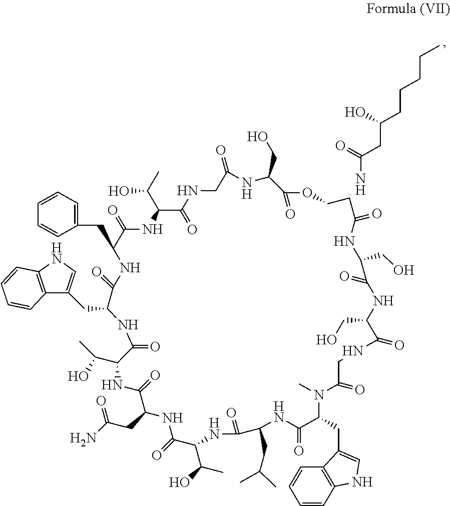

or a racemate, an enantiomer, a diastereomer, a pharmaceutically acceptable salt, or a derivative thereof.

In one embodiment, the compound of the present invention is a compound represented by Formula (VIII)

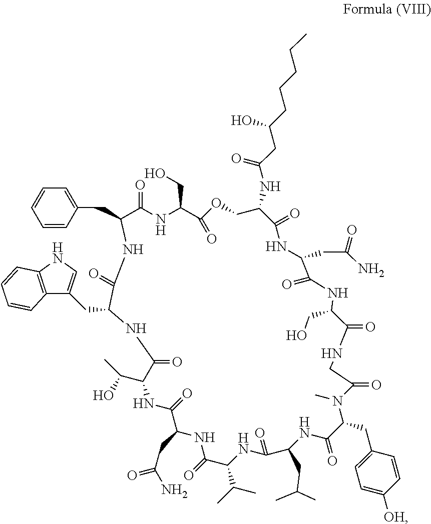

or a racemate, an enantiomer, a diastereomer, a pharmaceutically acceptable salt, or a derivative thereof.

In some embodiments, the compound represented by Formula (I) is a compound represented by Formula (Ia)

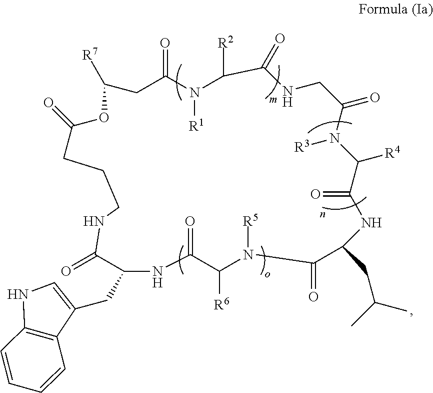

or a racemate, an enantiomer, a diastereomer, a pharmaceutically acceptable salt, or a derivative thereof.

In one embodiment, the compound represented by Formula (II) is a compound represented by Formula (IIa)

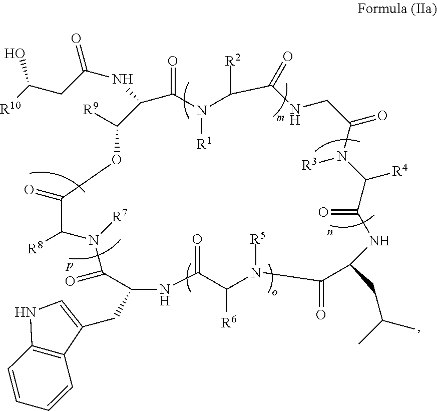

or a racemate, an enantiomer, a diastereomer, a pharmaceutically acceptable salt, or a derivative thereof.

The compounds described herein may form salts with acids or bases, and such salts are included in the present invention. The term “salts” embraces addition salts of free acids or free bases that are compounds of the invention.

In one aspect, the present invention relates, in part, to compositions comprising one or more compounds of the present invention. In some embodiments, the composition comprises one or more compounds having the structure of Formulae (I)-(VIII), or a racemate, an enantiomer, a diastereomer, a pharmaceutically acceptable salt, or a derivative thereof. In some embodiments, the composition is the pharmaceutical composition.

### Methods of Generating Compounds

In one aspect, the present invention relates, in part, to a method of generating one or more compounds of the present invention. In various embodiments, the compounds of the present invention can be generated using any method known to those of skill in the art. For example, in one embodiment, the compounds can be synthesized using any method known to those of skill in the art. For example, the compounds of the present invention may be synthesized using techniques well-known in the art of organic synthesis. The starting materials and intermediates required for the synthesis may be obtained from commercial sources or synthesized according to methods known to those skilled in the art.

In another embodiment, the present invention provides methods of generating the compounds of the present invention via isolated nucleic acids and vectors encoding the compound of the present invention. In one embodiment, when the nucleic acids and vectors are administered to a subject, they produce the compound of the present invention. In one embodiment, when the nucleic acids and vectors are administered to a subject, they produce an antibacterial effect.

The nucleic acid sequences include both the DNA sequence that is transcribed into RNA and the RNA sequence that is translated into a polypeptide. According to other embodiments, the polynucleotides of the invention are inferred from the amino acid sequence of the polypeptides of the invention. As is known in the art several alternative polynucleotides are possible due to redundant codons, while retaining the biological activity of the translated polypeptides.

It is to be understood explicitly that the scope of the present invention encompasses homologs, analogs, variants, fragments, derivatives and salts, including shorter and longer polynucleotides as well as polynucleotide analogs with one or more nucleic acid substitution, as well as nucleic acid derivatives, non-natural nucleic acids and synthetic nucleic acids as are known in the art, with the stipulation that these modifications must preserve the activity of the original molecule. The invention should be construed to include any and all isolated nucleic acids which are homologous to the nucleic acids described and referenced herein.

The skilled artisan would understand that the nucleic acids of the invention encompass a RNA or a DNA sequence comprising a sequence of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The coding sequence may comprise a codon that may allow more efficient transcription of the coding sequence in the host cell. In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Suitable host organisms include microorganisms, plant cells, and plants. The microorganism can be any microorganism suitable for expression of heterologous nucleic acids. In one embodiment the host organism of the invention is a eukaryotic cell. In another embodiment the host organism is a prokaryotic cell. In one embodiment, the host organism is a fungal cell such as a yeast or filamentous fungus. In one embodiment the host organism may be a yeast cell.

The host organism may also be a plant. plant or plant cell can be transformed by having a heterologous nucleic acid integrated into its genome, i.e., it can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a certain number of cell divisions.

In one embodiment, the engineered cell produces a compound of Formula (I). In some embodiments, the engineered cell produces at least one compound of Formula (I)-(VIII). For example, in one embodiment, the engineered cell produces a compound of Formula (I). In one embodiment, the engineered cell produces a compound of Formula (II).

In one embodiment, the engineered cell produces a compound of Formula (Ia). In some embodiments, the engineered cell produces at least one compound of Formula (Ia), (IIa), and (III)-(VIII). For example, in one embodiment, the engineered cell produces a compound of Formula (V). In one embodiment, the engineered cell produces a compound of Formula (VIII).

In one embodiment, the cell is a eukaryotic cell. In one embodiment, the cell may be a human cell, a non-human mammalian cell, a non-mammalian vertebrate cell, an invertebrate cell, an insect cell, a plant cell, a yeast cell, or a single cell eukaryotic organism. In one embodiment, the cell may be an adult cell or an embryonic cell (e.g., an embryo). In one embodiment, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others.

In one embodiment, the cell is a cell line cell. Non-limiting examples of suitable mammalian cells include Chinese hamster ovary (CHO) cells, baby hamster kidney (BHK) cells; mouse myeloma NSO cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C₃H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, human SW48 cells, human HCT116 cells, and human K562 cells. An extensive list of mammalian cell lines may be found in the American Type Culture Collection catalog (ATCC, Manassas, Va.).

In one embodiment, the cell can be a prokaryotic cell or a eukaryotic cell. In one embodiment, the cell is a prokaryotic cell. In one embodiment, the cell is a genetically engineered bacteria cell.

In one embodiment, the genetically engineered bacteria cell is a non-pathogenic bacteria cell. In some embodiments, the genetically engineered bacteria cell is a commensal bacteria cell. In some embodiments, the genetically engineered bacteria cell is a probiotic bacteria cell. In some embodiments, the genetically engineered bacteria cell is a naturally pathogenic bacteria cell that is modified or mutated to reduce or eliminate pathogenicity. Exemplary bacteria include, but are not limited to Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii.

In one embodiment, the host is a Streptomyces albus cell.

In some embodiments, the genetically engineered bacteria are Escherichia coli strain Nissle 1917 (E. coli Nissle), a Gram-negative bacterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

One of ordinary skill in the art would appreciate that the genetic modifications disclosed herein may be modified and adapted for other species, strains, and subtypes of bacteria.

### Treatment Methods

In one aspect, the invention provides methods of treating or preventing an infection in a subject in need thereof. In some embodiments, the method comprises administering to the subject an effective amount of a composition comprising at least one compound of the invention (e.g., at least one compound of Formula (I)-(VIII)). In some embodiments, the method comprises administering to the subject an effective amount of a composition comprising at least one nucleic acid of the invention.

In some embodiments, the method treats or prevents a bacterial infection. In one embodiment, the method treats or prevents a gram-positive bacterial infection. In one embodiment, the bacterial infection is resistant to antibiotics. For example, in one embodiment, the bacterial infection is resistant to one or more of, beta-lactams, including methicillin, oxacillin, or penicillin, tetracyclines, gentamicin, kanamycin, erythromycin, spectinomycin, and vancomycin.

Exemplary bacterial infections that may be treated by way of the present invention includes, but is not limited to, infections caused by bacteria from the taxonomic genus of Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and Yersinia. In some embodiments, the bacterial infection is an infection of Acinetobacter baumannii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterobacter cloacae, Enterococcus faecalis, Enterococcus faecium, Escherichia coli, Francisella tularensis, Haemophilus influenzae, Helicobacter pylori, Klebsiella species, Klebsiella pneumoniae, Legionella pneumophila, Leptospira interrogans, Leptospira santarosai, Leptospira weilii, Leptospira noguchii, Listeria monocytogenes, Morexella species, Moraxella osloensis, Mycobacterium leprae, Mycobacterium tuberculosis, Mycobacterium ulcerans, Mycoplasma pneumoniae, Neisseria gonorrhoeae, Neisseria meningitidis, Proteus species, Proteus vulgaris, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, or Yersinia pseudotuberculosis. In one embodiment, the bacterial infection is a Listeria monocytogenes infection.

In one embodiment, the bacterial infection is an infection of S. aureus USA300, S. aureus COL, S. aureus BAA-42, S. aureus NRS100, S. aureus NRS108, S. aureus NRS140, S. aureus NRS146, E. faecium VRE, E. faecium Com15, S. pneumoniae, S. mutans, B. subtilis, L. rhamnosus, E. coli, C. albicans, or C. neoformans.

Exemplary diseases caused by bacterial infections which may be treated using compositions of the present invention, include but are not limited to, bacterially mediated meningitis, sinus tract infections, pneumonia, endocarditis, pancreatitis, appendicitis, gastroenteritis, biliary tract infections, soft tissue infections, urinary tract infections, cystitis, pyelonephritis, osteomyelitis, bacteremia, Actinomycosis, Whooping cough, Secondary bacterial pneumonia, Lyme disease (B. burgdorferi), Relapsing fever, Brucellosis, Enteritis, bloody diarrhea, Guillain-Barre syndrome, Atypical pneumonia, Trachoma, Neonatal conjunctivitis, Neonatal pneumonia, Nongonococcal urethritis (NGU), Urethritis, Pelvic inflammatory disease, Epididymitis, Prostatitis, Lymphogranuloma venereum (LGV), Psittacosis, Botulism: Mainly muscle weakness and paralysis, Pseudomembranous colitis, Anaerobic cellulitis, Gas gangrene Acutefood poisoning, Tetanus, and Diphtheria.

However, the invention should not be limited to only treating bacterial infection. The invention encompasses compounds having an antimicrobial activity including but not limited to antibacterial, antimycobacterial, antifungal, antiviral and the likes.

In one aspect, the invention provides methods of killing a bacterial cell or inhibiting the grown of a bacterial cell. In some embodiments, the method comprises administering to the cell an effective amount of a composition comprising at least one compound of the invention. In some embodiments, the method comprises administering to the cell an effective amount of a composition comprising at least one nucleic acid of the invention. In one embodiment the bacterial cell is a gram positive bacterial cell. In one embodiment, the bacterial cell is resistant to antibiotics. For example, in one embodiment, the bacterial cell is resistant to one or more of, beta-lactams, including methicillin, oxacillin, or penicillin, tetracyclines, gentamicin, kanamycin, erythromycin, spectinomycin, and vancomycin.

In another aspect, the invention provides compositions and methods for treating and/or preventing a disease or disorder related to the detrimental growth and/or proliferation of a bacterial cell in vivo, ex vivo or in vitro. In certain embodiments, the method comprises administering a composition comprising an effective amount of a composition provided by the invention to a subject, wherein the composition is effective in inhibiting or preventing the growth and/or proliferation of a bacterial cell. In certain embodiments, the bacterial cell is a Gram-positive bacterial cell, e.g., a bacteria of a genera such as Staphylococcus, Streptococcus, Enterococcus, (which are cocci) and Bacillus, Corynebacterium, Nocardia, Clostridium, Actinobacteria, and Listeria (which are rods and can be remembered by the mnemonic obconical), Mollicutes, bacteria-like Mycoplasma, Actinobacteria.

In certain embodiments, the bacterial cell is a Gram-bacteria cell, e.g., a bacteria of a genera such as Acinetobacter, Citrobacter, Enterobacter, Enterococcus, Escherichia, Helicobacter, Hemophilus, Klebsiella, Legionella, Moraxella, Neisseria, Proteus, Pseudomonas, Salmonella, Staphylococcus, and Yersinia. The compounds as described herein and compositions comprising them may thus be for use in the treatment of bacterial infections by the above-mentioned Gram+ or Gram− bacteria.

In one embodiment, the method further comprises administering a second therapeutic agent. In one embodiment, the second therapeutic agent is an antibiotic agent. In one embodiment, the compound of the invention and the at least one additional antibiotic agent act synergistically in preventing, reducing or disrupting microbial growth.

Non-limiting examples of the at least one additional antibiotic agents include levofloxacin, doxycycline, neomycin, clindamycin, minocycline, gentamycin, rifampin, chlorhexidine, chloroxylenol, methylisothizolone, thymol, α-terpineol, cetylpyridinium chloride, hexachlorophene, triclosan, nitrofurantoin, erythromycin, nafcillin, cefazolin, imipenem, astreonam, gentamicin, sulfamethoxazole, vancomycin, ciprofloxacin, trimethoprim, rifampin, metronidazole, clindamycin, teicoplanin, mupirocin, azithromycin, clarithromycin, ofoxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linexolid, temafloxacin, tosufloxacin, clinafloxacin, sulbactam, clavulanic acid, amphotericin B, fluconazole, itraconazole, ketoconazole, nystatin, penicillins, cephalosporins, carbepenems, beta-lactams antibiotics, aminoglycosides, macrolides, lincosamides, glycopeptides, tetracylines, chloramphenicol, quinolones, fucidines, sulfonamides, trimethoprims, rifamycins, oxalines, streptogramins, lipopeptides, ketolides, polyenes, azoles, echinocandines, and any combination thereof.

In one embodiment, the compositions of the invention find use in removing at least a portion of or reducing the number of microorganisms and/or biofilm-embedded microorganisms attached to the surface of a medical device or the surface of a subject's body (such as the skin of the subject, or a mucous membrane of the subject, such as the vagina, anus, throat, eyes or ears). In one embodiment, the compositions of the invention find further use in coating the surface of a medical device, thus inhibiting or disrupting microbial growth and/or inhibiting or disrupting the formation of biofilm on the surface of the medical device. The compositions of the invention find further use in preventing or reducing the growth or proliferation of microorganisms and/or biofilm-embedded microorganisms on the surface of a medical device or on the surface of a subject's body. However, the invention is not limited to applications in the medical field. Rather, the invention includes using a compound or an analog thereof as an antimicrobial and/or antibiofilm agent in any setting.

The composition of the invention may be administered to a patient or subject in need in a wide variety of ways, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the composition is administered systemically to the subject. In one embodiment, the compositions of the present invention are administered to a patient by i.v. injection. In one embodiment, the composition is administered locally to the subject. In one embodiment, the compositions of the present invention are administered to a patient topically. Any administration may be a single application of a composition of invention or multiple applications. Administrations may be to single site or to more than one site in the individual to be treated. Multiple administrations may occur essentially at the same time or separated in time.

In one aspect, the compositions of the invention may be in the form of a coating that is applied to the surface of a medical device or the surface of a subject's body. In one embodiment, the coating prevents or hinders microorganisms and/or biofilm-embedded microorganisms from growing and proliferating on at least one surface of the medical device or at least one surface of the subject's body. In another embodiment, the coating facilitates access of antimicrobial agents to the microorganisms and/or biofilm-embedded microorganisms, thus helping prevent or hinder the microorganisms and/or biofilm-embedded microorganisms from growing or proliferating on at least one surface of the medical device or at least one surface of the subject's body. The compositions of the invention may also be in the form of a liquid or solution, used to clean the surface of medical device or the surface of a subject's body, on which microorganisms and/or biofilm-embedded microorganisms live and proliferate. Such cleaning of the medical device or body surface may occur by flushing, rinsing, soaking, or any additional cleaning method known to those skilled in the art, thus removing at least a portion of or reducing the number of microorganisms and/or biofilm-embedded microorganisms attached to at least one surface of the medical device or at least one surface of the subject's body.

Subjects to which administration of the pharmaceutical compositions of the invention is contemplated include, but are not limited to, humans and other primates, mammals including but not limited to non-human mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

### Dosage and Formulation (Pharmaceutical Compositions)

The invention also encompasses the use of pharmaceutical compositions comprising a compound of the invention, a nucleic acid of the invention, or salts thereof. Such a pharmaceutical composition may comprise of at least one a compound of the invention, a nucleic acid of the invention, or salts thereof in a form suitable for administration to a subject, or the pharmaceutical composition may comprise at least one a compound of the invention, a nucleic acid of the invention, or salts thereof, and one or more pharmaceutically acceptable carriers, one or more additional ingredients, or some combination of these. The compound or nucleic acid of the invention may be present in the pharmaceutical composition in the form of a physiologically acceptable salt, such as in combination with a physiologically acceptable cation or anion, as is well known in the art.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art

The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

In one embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

Typically, dosages which may be administered in a method of the invention to a mammal, preferably a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 5 mg per kilogram of body weight of the mammal.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The compounds and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

The present invention also provides pharmaceutical compositions comprising one or more of the compositions described herein. Formulations may be employed in admixtures with conventional excipients, i.e., pharmaceutically acceptable organic or inorganic carrier substances suitable for administration to subject. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the HMW-HA or other composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Menaquinone-Binding Natural Products are a Structurally Diverse Class of Antibiotics with In Vivo Activity Against Multidrug-Resistant Pathogens

Three closely related nonribosomal peptide synthetase (NRPS) derived bacterial cyclic lipopeptides (lysocin E, WAP-8294A2 and WBP-29479A1) have been shown to kill bacteria by binding directly to MK to induce membrane disruption and rapid cell lysis (FIG. 1) (Hamamoto et al., 2015, Nat. Chem. Biol. 11, 127-133; Itoh et al., 2018, J. Org. Chem. 83, 6924-6935; Sang et al., 2019, Org. Lett. 21, 6432-6436). The search for additional bacterially produced MK-binding antibiotics (MBAs) is limited by the fact that most of the biosynthetic diversity in the global microbiome remains functionally inaccessible. This is due both to the inability to culture most bacteria and to the fact that only a small subset of biosynthetic gene clusters (BGCs) found in cultured bacteria is expressed in laboratory fermentation studies (Rutledge et al., 2015, Nat. Rev. Microbiol. 13, 509-523; Crits-Christoph, 2018, Nature 558, 440-444). While these factors limit direct functional screening for additional MBAs, next generation sequencing methods are revealing large numbers of previously inaccessible bacterial BGCs from both cultured bacteria and diverse metagenomes (Libis, Nat. Commun. 10, 3848 (2019); Kalkreuter, Trends Pharmacol. Sci. 41, 13-26 (2020); Nayfach, Nat. Biotechnol. 39, 499-509 (2020)). The genetic information contained in a BGC has historically been decoded using biological processes (i.e., transcription, translation and biosynthesis). This paradigm is limited by the fact that most BGCs are natively silent in laboratory fermentation studies, and even the best activation strategies can only activate a small fraction of BGCs in native or heterologous organisms (Rutledge, Nat. Rev. Microbiol. 13, 509-523 (2015)). The increasing accuracy of structural predictions derived from the bioinformatic analysis of BGCs presents the alternative possibility of accessing the metabolite encoded by a BGC using total chemical synthesis of its bioinformatically predicted product (i.e., a synthetic bioinformatic natural product, syn-BNP) (Chu, Nat. Chem. Biol. 12, 1004-1006 (2016); Chu, J. Am. Chem. Soc. 141, 15737-15741 (2019)).

The present studies took two orthogonal bioinformatic approaches to guide the discovery of bacterial BGCs that encode structurally novel classes of MBAs (FIG. 1). First, in a culture-independent approach, sequence homology was used to identify predicted MBA BGCs from diverse soil metagenomes. A detailed analysis of BGCs that arose from this study led us to propose a minimal MK-binding motif (GXLXXXW). Next, in the first search of its kind, a large database of bioinformatically predicted natural product structures was screened for this proposed minimal MK-binding motif to identify additional potential MBA BGCs in sequenced bacterial genomes. Total chemical synthesis of the structures predicted to arise from the BGCs that identified resulted in six structurally diverse MBAs. All six MBAs were broadly active against MDR Gram-positive pathogens. Notably, it was shown for the first time that a subset of MBAs is active against MDR Mycobacterium tuberculosis (Mtb) both in vitro and in a macrophage assay, defining a new anti-Mtb mode of action. Among the identified antibiotics, four fell into two new structural classes. Antibiotics from both new structural classes proved effective against methicillin-resistant Staphylococcus aureus (MRSA) in a murine peritonitis-sepsis model, thus providing two new MBAs for use in the development of antibiotics with different modes of action and activity against MDR pathogens. The general approach presented here of searching a database of structures bioinformatically sequenced to identify BGCs that encode molecules with specific desired features is broadly applicable to the search for bioactive small molecules.

### Results

### Identification of BGCs Predicted to Encode MBAs

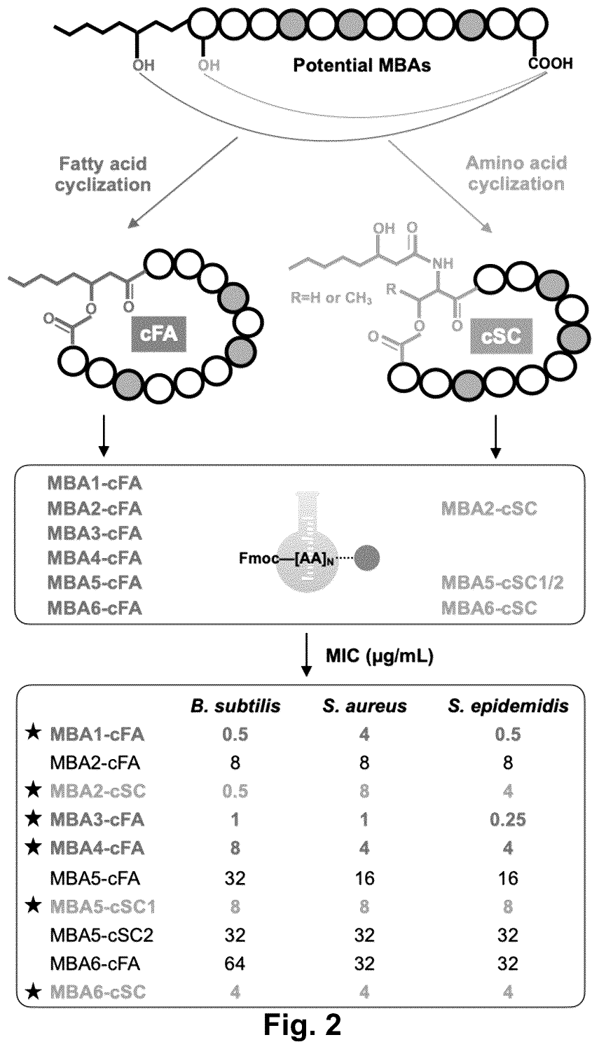

Metagenomic search for BGCs that are predicted to encode new MBAs: The search for BGCs that might encode MBAs began by looking at NRPS adenylation (A)-domain sequence data generated from soil metagenomic libraries. As part of the ongoing soil metagenome-guided natural product discovery program, a collection of saturating cosmid-based soil metagenomic libraries was created for use in targeted BGC discovery studies (Hover, Nat. Microbiol. 3, 415-422 (2018); Peek, Nat. Commun. 9, 4147 (2018).; Charlop-Powers, eLife 4, e05048 (2015); Lemetre, Proc. Natl. Acad. Sci. U.S.A. 114, 11615-11620 (2017).). The construction and screening of these libraries for A-domain sequences associated with a specific natural product family have been described in detail before (Hover, Nat. Microbiol. 3, 415-422 (2018); Lemetre, Proc. Natl. Acad. Sci. U.S.A. 114, 11615-11620 (2017)). Briefly, for each library, DNA extracted directly from soil (environmental DNA, eDNA) was used to construct >2×10⁷ unique cosmid clones in Escherichia coli. Each library was arrayed as a set of sub-pools containing ˜5-25,000 unique cosmids each. DNA from each sub-pool was used as template in a PCR reaction with a unique set of barcoded A-domain degenerate primers. The resulting amplicons were sequenced using Illumina technology to generate a database of A-domain sequences (i.e., Natural Product Sequence Tags, NPSTs) that can be used to track BGCs of interest to specific library sub-pools. Using the environmental surveyor of natural product diversity (eSNaPD) software package (Reddy, Chem. Biol. 21, 1023-1033 (2014)), each A-domain from a BGC that encodes one of the three known MBAs was compared to all of the library derived A-domain NPSTs. Known MBAs shared six positionally conserved amino acids: L-Ser-3, Gly-4, D-Phe-5, L-Leu-6, L-Glu-8 and D-Trp-10 (FIG. 1). NPSTs that returned low e-values (<10⁻¹² to <10⁻⁶⁰) for the A-domains that install one of these six conserved residues were used to generate six A-domain-specific phylogenetic trees (FIG. 12). eDNA cosmid clones containing BGCs associated with A-domains that fell into the same or a closely related clade as an A-domain from known MBA BGCs were recovered from the appropriate library sub-pools. Fully sequenced and annotated eDNA derived NRPS BGCs were analyzed for the potential to encode MBA-like peptides. The linear peptide encoded by each eDNA derived BGC was predicted using the 10 amino acid residues that line each A-domain substrate binding pocket (e.g., A-domain signature sequence) (Stachelhaus, Chem. Biol. 6, 493-505 (1999); Blin, Nucleic Acids Res. 47, W81-W87 (2019)). Based on this analysis no predicted peptides contained all six residues that were conserved among known MBAs. In three cases however, where the eDNA derived BGC showed a similar gene organization to that seen in known MBA BGCs, the predicted peptide products shared some sequence similarity to known MBAs (FIG. 13), leading us to explore the possibility that the structures predicted to arise from these BGCs might be MBAs. As shown in the bioactivity analysis presented below each of these predicted peptides does in fact represent a new MBA (FIG. 2).

GXLXXXW Motif Search of Sequenced Genomes for BGCs that are Predicted to Encode MBAs:

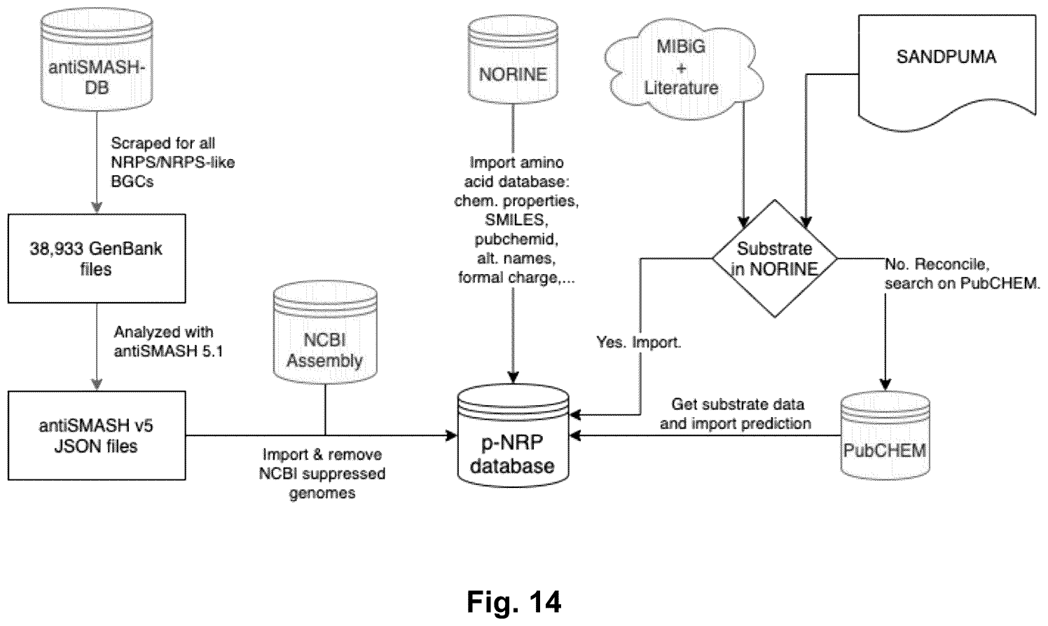

In a second round of screening, sequenced bacterial genomes were evaluated to see if BGCs that might encode additional MBAs could be identified. For this study BGCs from ˜10,000 bacterial genomes were analyzed. The A-domain substrate binding pockets from NRPS BGCs in these genomes were compared to a manually curated list of A-domain signature sequences from characterized BGCs (see Methods). Based on the A-domain substrate predictions that arose from this analysis a database of linear peptides was generated that were predicted to arise from >36,000 NRPS BGCs (FIG. 1 and FIG. 14). The initial search failed to identify any BGCs that might encode a peptide that contains the same six amino acid pattern that is seen in previously characterized MBAs. By including metagenome derived MBA sequences in this study, the conserved residues observed across MBAs was able to be reduced from the initial six residues to a proposed minimal MK-binding motif of just three residues (Gly-X-Leu-X-X-X-Trp, GXLXXXW) (FIG. 1). A search of the database of predicted NRPS derived linear peptides with the simpler GXLXXXW motif identified one BGC in the genome of Dyella mobilis indicating that it might encode a potential MBA (FIG. 1 and FIG. 15). As NRPS BGCs are often truncated in genomes assembled using short read sequence data (e.g., Illumina sequencing), the database was also searched for sub-sequences of the GXLXXXW motif GXL was found in 22 peptides and LXXXW was only found in the peptide from D. mobilis (FIG. 7). A manual examination the remaining amino acids in these 22 peptides identified two additional examples where peptides shared additional amino acids with newly predicted MBAs. One example was from a truncated BGC in Dyella tabacisoli and the other was found in partially sequenced NRPS BGCs from four different Paracoccus strains (FIG. 7 and FIG. 15). To access the complete non-ribosomal peptide (NRP) sequence encoded by each of these BGCs, the genomes of D. tabacisoli KCTC 62035 and Paracoccus alcaliphilus ATCC 51199 were re-sequenced. In both cases A-domain analysis of fully sequenced BGCs predicted they would encode GXLXXXW containing peptides that might represent MBAs (FIG. 1).

### Prediction and Synthesis of 10 Potential MBA BGC Products

In total, six BGCs were identified that might encode MBAs. Traditional natural product discovery methods that rely on biological systems to decode the information present in a BGC are hindered by a lack of BGC expression in laboratory fermentation studies, as well as the time consuming process of isolating and structurally characterizing molecules from bacterial fermentation broths. In cases where the final product of a BGC can be bioinformatically predicted with confidence, total chemical synthesis of this product can provide an alternative and potentially more straightforward means of accessing the metabolite encoded by the BGC (Chu, Nat. Chem. Biol. 12, 1004-1006 (2016); Chu, J. Am. Chem. Soc. 141, 15737-15741 (2019)). Each potential MBA BGC contains two large NRPS genes with a condensation start domain that is predicted to initiate NRPS biosynthesis with a fatty acid (Chen, RCS Adv. 5, 105753-105759 (2015)). As described above, A-domain substrate specificity analysis allowed us to predict with high confidence the amino acid incorporated by every A-domain found in these BGCs (FIG. 16). With the exception of the MBA6 BGC, no BGCs were predicted to encode tailoring enzymes (FIG. 1 and FIG. 15). The dioxygenase encoded by the MBA6 BGC was expected to be involved in the hydroxylation of the Asn incorporated as the second amino acid in the peptide (Zhang, Antimicrob. Agents Chemother. 55, 5581-5589 (2011)). In the known MBA WAP-8294A2, the hydroxyl group on the Asn at the same position (D-OHAsn-2) is not required for anti-bacterial activity (Itoh, J. Org. Chem. 83, 6924-6935 (2018); Chen, Bioorg. Med. Chem. 28 (2020).). The unmodified linear lipopeptide produced by each NRPS system was therefore predicted to be the direct precursor to the final biologically active cyclic lipopeptide produced by each BGC. As the product of each potential MBA BGC could be bioinformatically predicted with confidence, and in each case the linear NRPS derived peptide appeared to be the direct precursor to the final cyclic peptide product, total chemical synthesis was likely to be the most straightforward means of accessing the bioactive metabolites encoded by the six potential MBA BGCs identified.

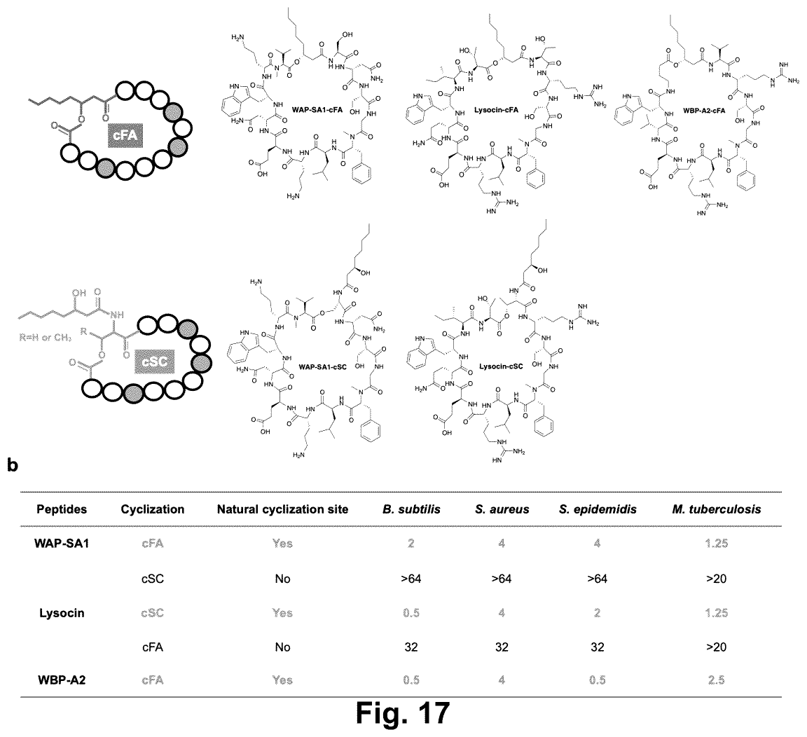

Two key structural features that cannot be bioinformatically predicted with high confidence from the primary sequence of these BGCs are the exact lipid used to initiate biosynthesis, and the mode of peptide cyclization. Distinct from many other cyclic lipodepsipeptides (Bionda, Future Med. Chem. 5, 1311-1330 (2013)), changes in the fatty acid tail of known MBAs did not result in significant differences in anti-bacterial activity (Hamamoto, Nat. Chem. Biol. 11, 127-133 (2015); Kato, J. Antibiot. 51, 929-935 (1998)). (R)-3-hydroxy-octanoic acid, which is found in two of the three known MBAs (WAP-8294A and lysocins) was used in the synthesis of all predicted BGC products (Hamamoto, Nat. Chem. Biol. 11, 127-133 (2015); Kato, J. Antibiot. 51, 929-935 (1998)). The N-acylated linear peptide corresponding to the bioinformatically predicted product of each potential MBA BGC was generated by Fmoc-based solid-phase peptide synthesis (SPPS). (R)-3-hydroxy-octanoic acid derivatized linear peptides can either be cyclized through the β-hydroxyl of the fatty acid (fatty acid cyclized, cFA) or through a nucleophilic amino acid side chain (side chain cyclized, cSC) (FIG. 2). When no nucleophilic side chain was present in the peptide, only a fatty acid cyclized derivative was produced from the linear peptide (MBA1, MBA3 and MBA4). However, when the first amino acid was predicted to contain a nucleophilic side chain (i.e., a serine or threonine) both fatty acid cyclized and amino acid side chain cyclized analogs were synthesized from the linear peptide (MBA2, MBA5 and MBA6). In the case of MBA5 which was predicted to contain amino acids with a nucleophilic side chain at the first two positions, two distinct amino side chain cyclized peptides were generated. In total 10 synthetic bioinformatic natural products (syn-BNPs) were generated for bioactivity screening (FIG. 2). For use as controls, (R)-3-hydroxy-octanoic acid analogs of the three known MBAs were synthesized (FIG. 17, WAP-SA1, lysocin and WBP-A2).

### Anti-Microbial Spectrum

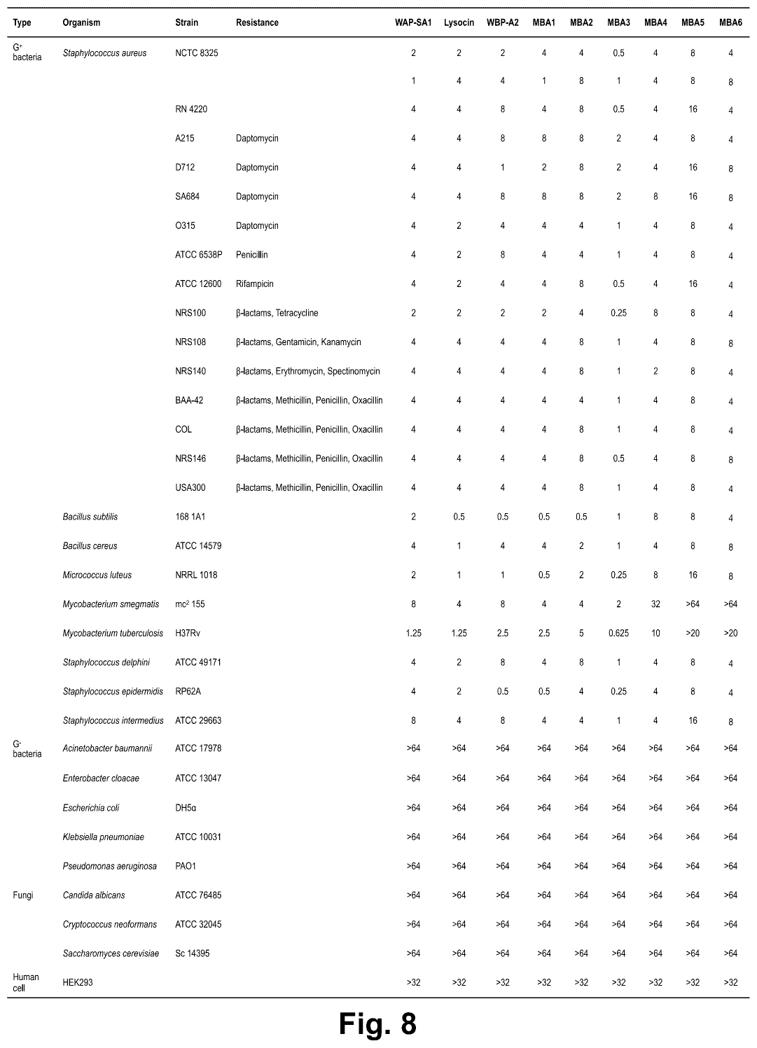

MK binding antibiotics are expected to have Gram-positive antibacterial activity because MK plays an important role in the electron transport system of Gram-positive bacteria (Johnston et al., 2020, Curr. Opin. Struct. Biol. 65, 33-41; Boersch et al., 2018, RSC Adv. 8, 5099-5105). Each syn-BNP was initially tested against a small number of Gram-positive bacteria (e.g., Bacillus subtilis, Staphylococcus aureus and Staphylococcus epidermidis) to determine which had antibacterial activity (FIG. 2 and FIG. 8). In all cases where differentially cyclized structures (MBA2, MBA5 and MBA6) were synthesized from a single linear peptide, cyclization through the serine or threonine at the first position showed the most potent antibiotic activity. These more active structures are the likely products of these BGCs, and they were therefore used in all of the subsequent studies. “Unnaturally cyclized” versions of two known MBA analogs (lysocin and WAP-SA1) were also synthesized and in both cases these structures showed a comparable decrease or loss of anti-bacterial activity (FIG. 17). In the case of syn-BNPs predicted from the MBA1, MBA3 and MBA4 BGCs, the only possible cyclization pattern was through the 3-hydroxy of the N-terminal fatty acid. All three compounds showed good Gram-positive antibiosis (FIG. 2). Ultimately, this analysis resulted in six new and structurally diverse cyclic lipopeptide antibiotics (MBA1 through 6) that are only linked by a shared GXLXXXW motif that was associated with MK binding as a mode of action.

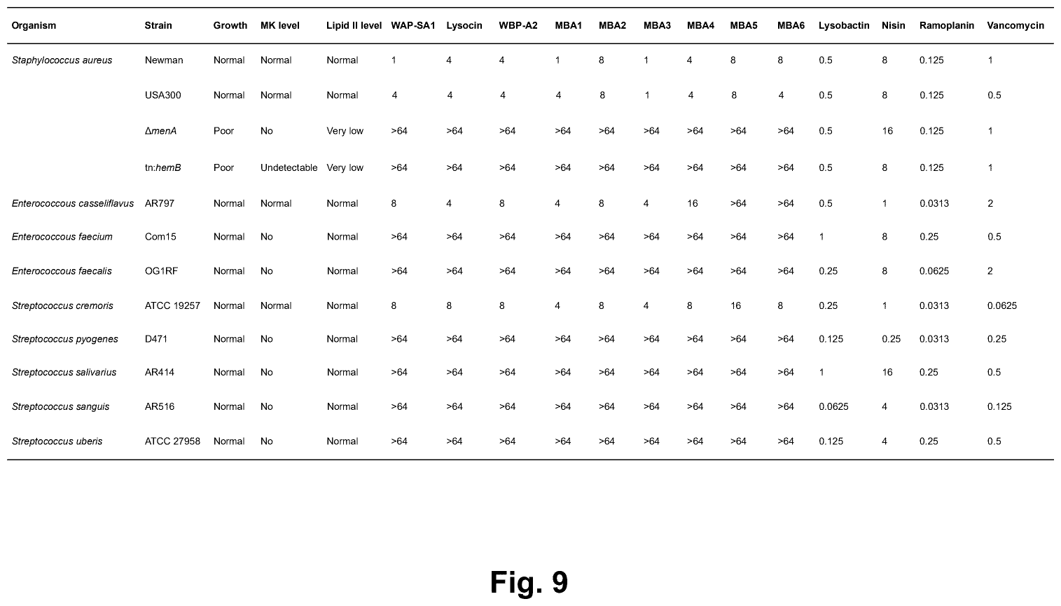

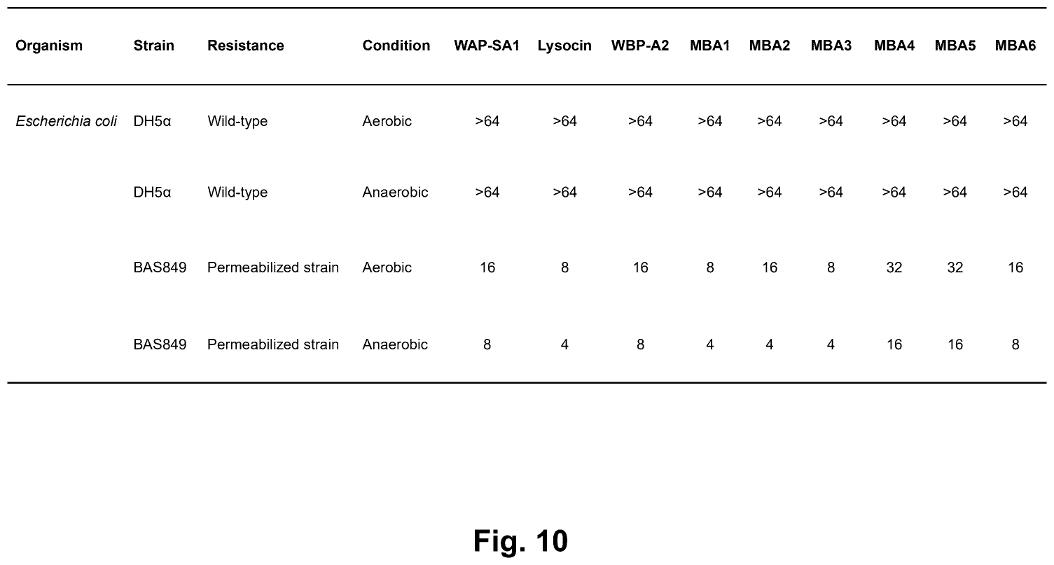

All six syn-BNPs were broadly active against Gram-positive bacteria. Against a panel of S. aureus strains that are resistant to diverse clinically relevant antibiotics, the MBA MICs ranged from 0.25 to 8 μg/mL. MBA3, which was predicted from a metagenome derived BGC, was the most potent antibiotic with the MICs against S. aureus strain ranging from 0.25 to 2 μg/mL. A subset of these syn-BNPs, MBA3 in particular, were active against Mtb (FIG. 8). Lysocin E has been reported to be active against Mycobacterium smegmatis (Yagi, J. Antibiot. 70, 685-690 (2017)), but to the best of the knowledge, this is the first report of an MBA having anti-Mtb activity. To begin to explore the relevance of MK to the activity of these antibiotics in more detail their MICs against a collection of Enterococcus and Streptococcus spp. that either natively produce or do not produce MK were determined. The syn-BNPs were active against the two tested MK-producing strains (e.g., Enterococcus casseliflavus and Streptococcus cremoris) but were inactive against all six tested MK-deficient Enterococcus and Streptococcus strains (FIG. 9) (Collins, J. Gen. Microbiol. 114, 27-33 (1979); Huycke, Mol. Microbiol. 42, 729-740 (2001)). In contrast, all four known tested lipid II binders (lysobactin, nisin, ramoplanin and vancomycin) were active against all eight Enterococcus or Streptococcus strains (FIG. 9) (Müller, Nat. Prod. Rep. 34, 909-932 (2017); Malin, Infect. Drug Resist. 12, 2613-2625 (2019)). In broader bioactivity screening, no syn-BNP showed activity against any wild-type Gram-negative bacteria or fungi that were tested (FIG. 8). Against outer membrane permeabilized E. coli (E. coli BAS849) syn-BNP MICs ranged from 8 to 32 μg/mL (FIG. 10) (Sampson, Genetics 122, 491-501 (1989)). In Gram-negative bacteria MK is produced under both aerobic and anaerobic growth conditions; however, it is produced at higher levels in anaerobic growth conditions because of its key role in respiration (Meganathan, Vitam. Horm. 61, 173-218 (2001); Meganathan, EcoSal Plus 3, 10.1128/ecosalplus.3.6.3.3 (2009).). Under anaerobic growth conditions syn-BNP MICs against E. coli BAS849 were 2 to 4-fold lower than under aerobic conditions (FIG. 10). The absence of activity against E. coli, and likely other Gram-negative bacteria, appears to be due to the inability of syn-BNPs to cross the outer membrane. Collectively, these spectrum of activity data provided the first experimental evidence that all six new antibiotics were likely MBAs.

### Mode of Action Studies

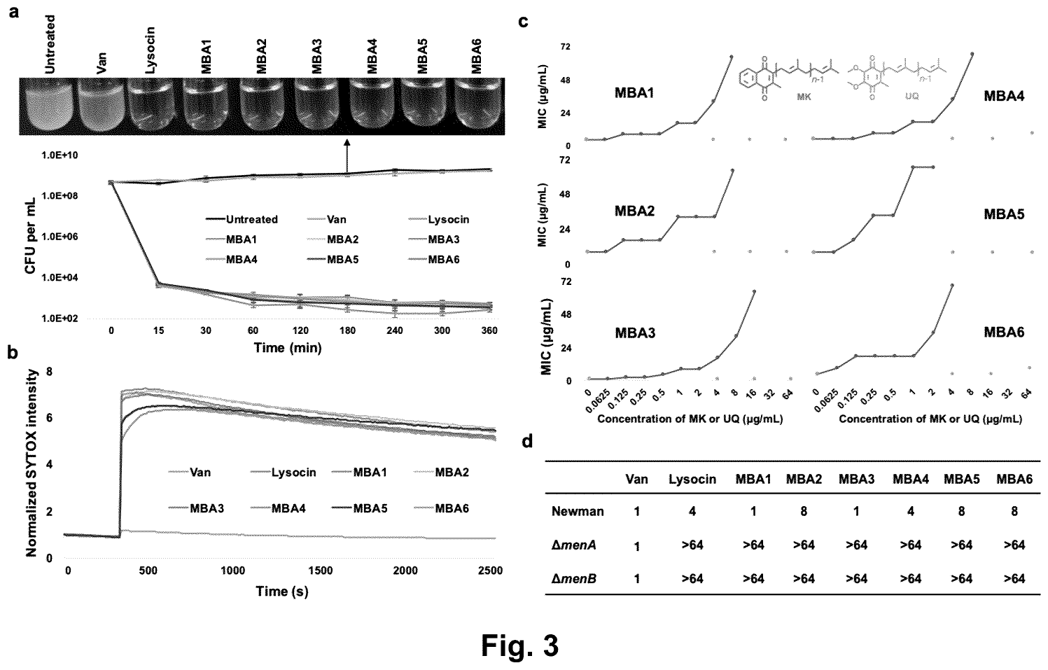

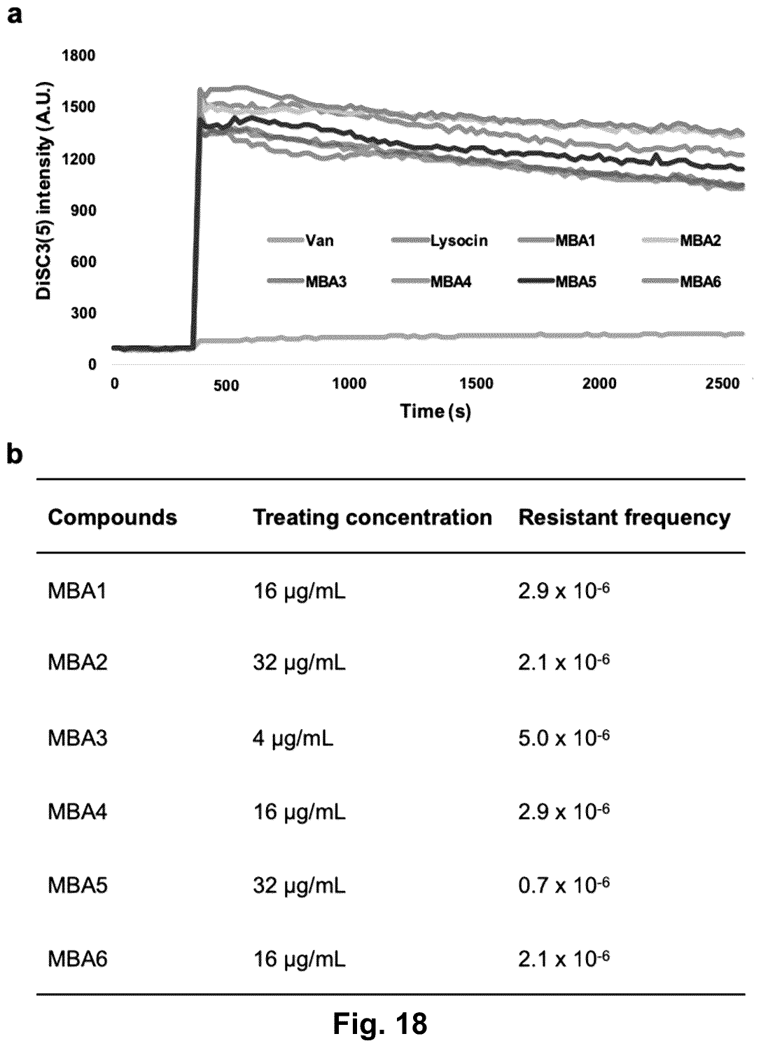

Known MBAs cause rapid cell death due to membrane lysis (Hamamoto, Nat. Chem. Biol. 11, 127-133 (2015); Itoh, J. Org. Chem. 83, 6924-6935 (2018)). Each active syn-BNP was therefore tested for the ability to lyse S. aureus. As shown in FIG. 3a, when added to S. aureus cultures each syn-BNP antibiotic caused a rapid decrease in the number of viable cells. Membrane depolarization and cell lytic activities by all six syn-BNPs were confirmed using 3,3′-dipropylthiadicarboncyanine iodide [DiSC₃(5)] and SYTOX fluorescence assays, respectively (FIG. 3b and FIG. 18a).

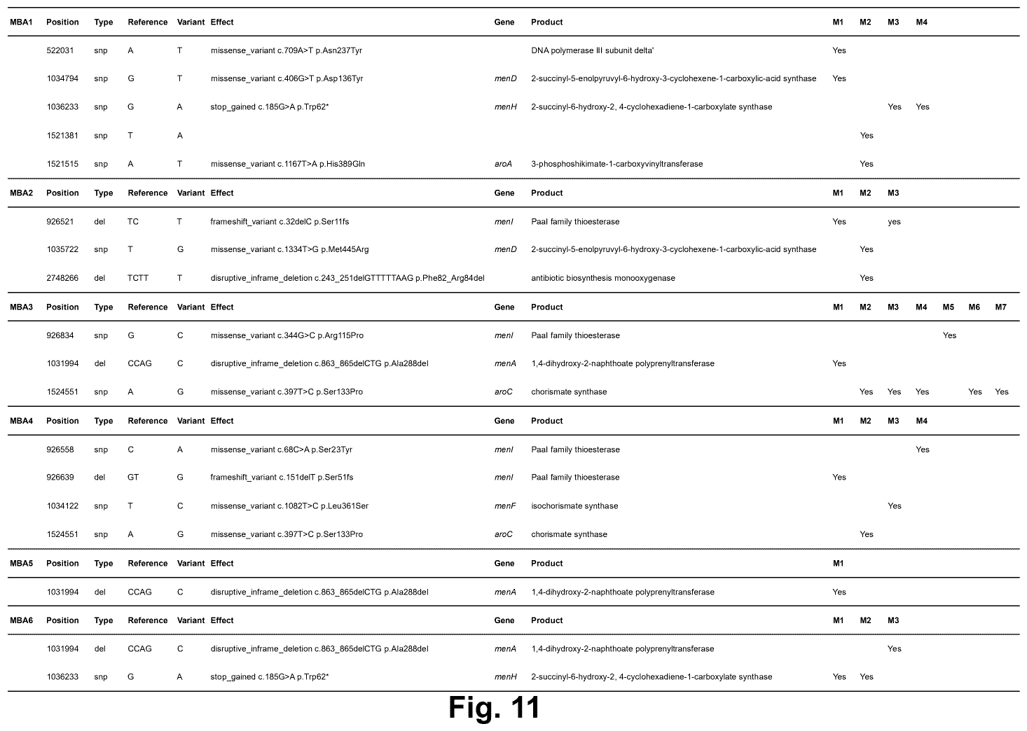

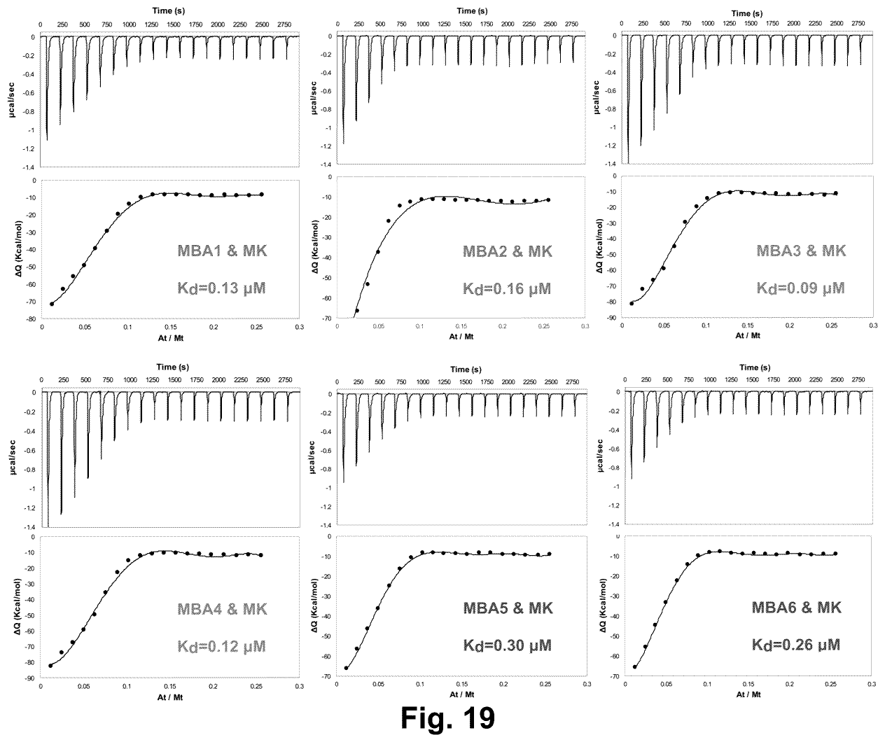

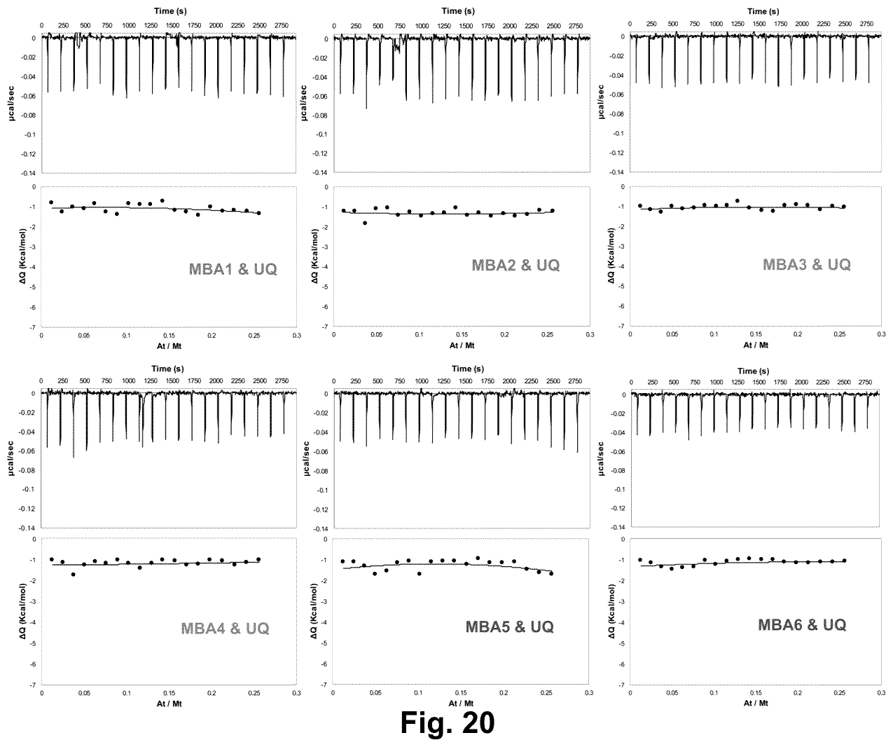

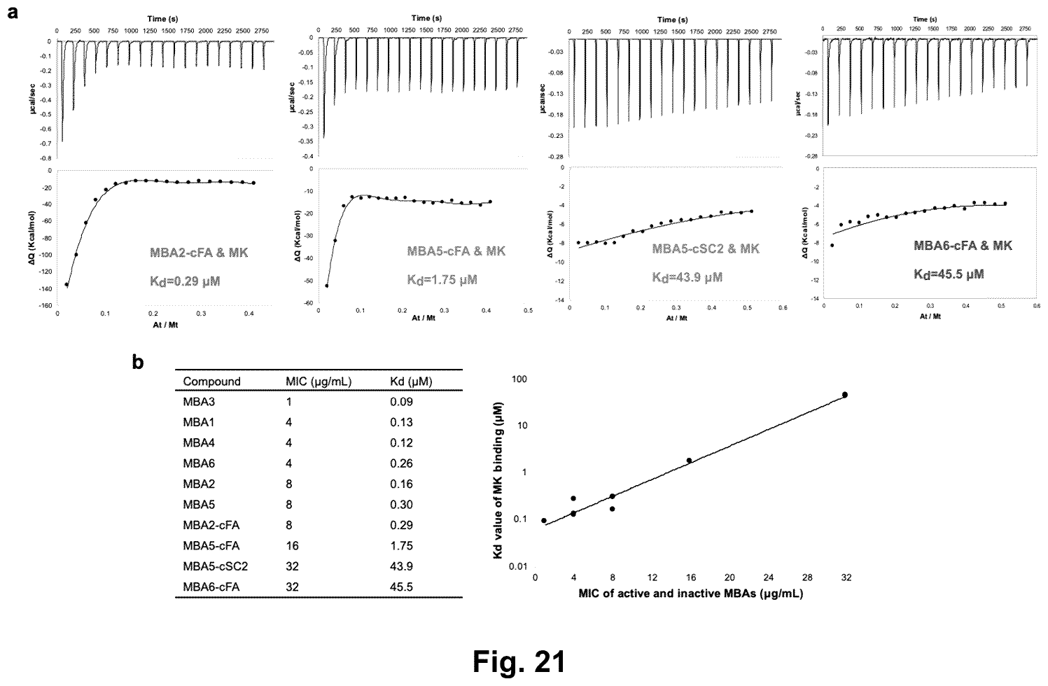

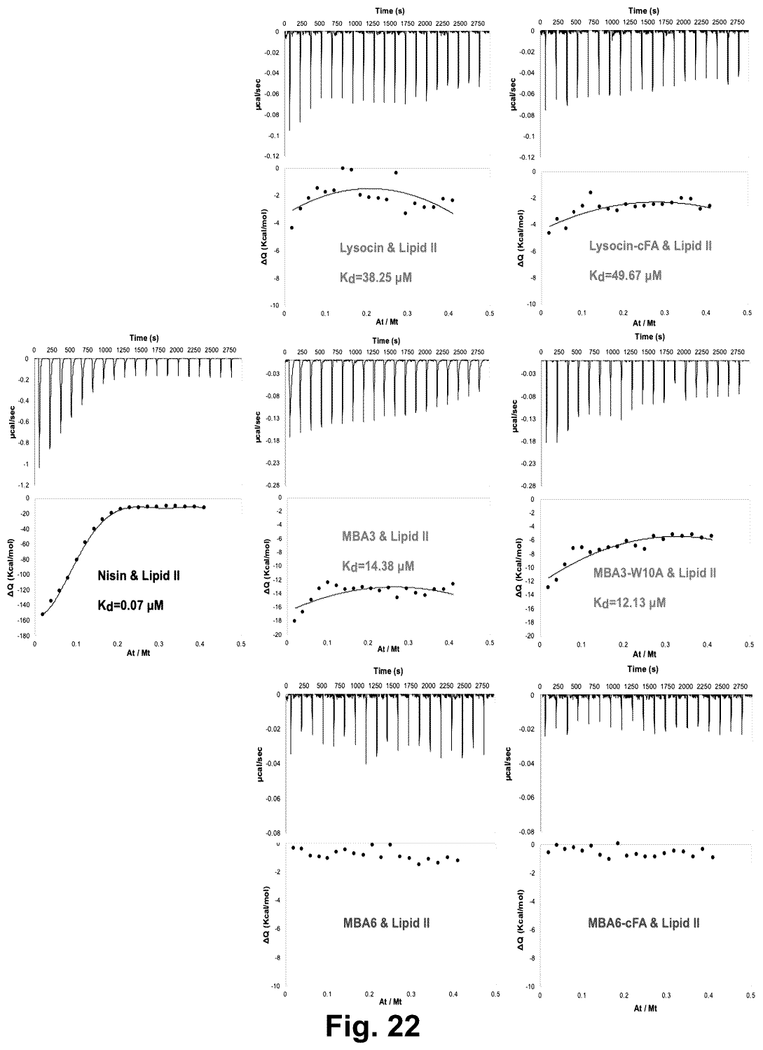

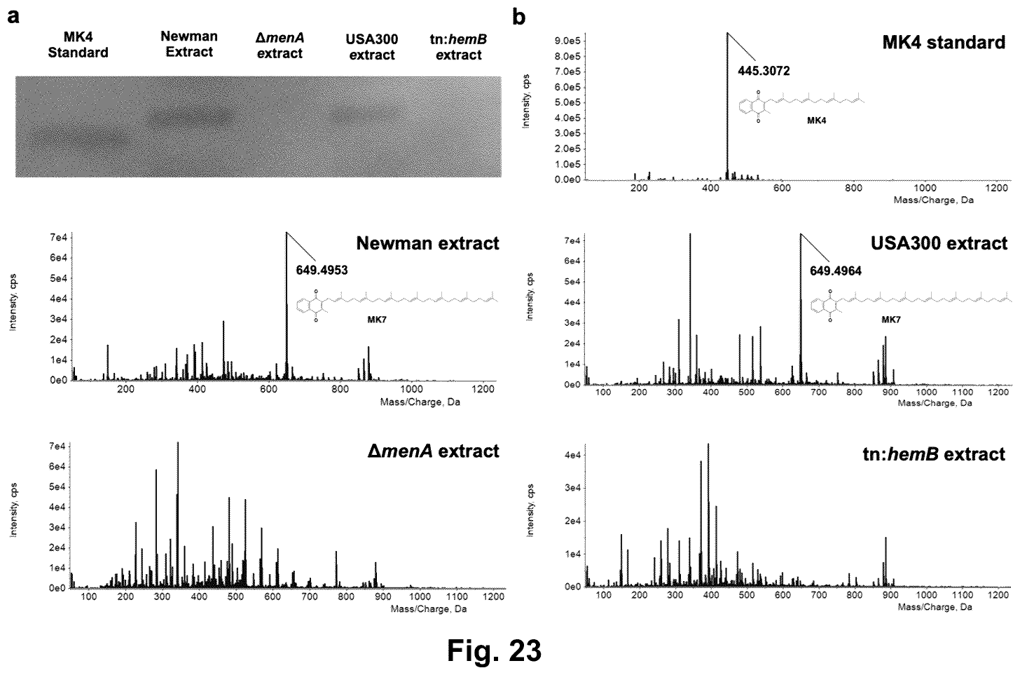

The relevance of MK to each syn-BNP's antibiosis activity was examined in four ways: 1) assayed for MK's ability to suppress antibiosis, 2) tested for antibiosis against MK biosynthesis knockout strains, 3) raised resistant mutants to each antibiotic and 4) accessed the binding of each active syn-BNP to MK directly using isothermal titration calorimetry (ITC). When MK was added to the assay medium, the MIC of each syn-BNP against S. aureus increased in a dose-dependent manner. The related structure, ubiquinone (UQ), had no effect on the antibiotic activity of any syn-BNP (FIG. 3c). Two different S. aureus MK biosynthesis knockout strains (ΔmenA and ΔmenB) were used to test whether antibiosis was dependent on native production of MK. In Gram-positive bacteria MK is used as an electron donor in respiration. Although in the absence of MK S. aureus cannot respire, they can survive by generating ATP from substrate phosphorylation. Both menA and menB deletion strains are viable but they have small colony variant (SCV) phenotypes because they can only generate ATP from substrate phosphorylation (Wakeman, Mol. Microbiol. 86, 1376-1392 (2012)). All six syn-BNPs were inactive (MIC>64 μg/mL) against both S. aureus strains (FIG. 3d). Furthermore, S. aureus (USA300) mutants that could grow on 4× the MIC of each antibiotic were selected. At 4× each syn-BNP's MIC resistant mutants appeared at a frequency of 0.7-5×10⁻⁶ for all syn-BNPs (FIG. 18b). For each antibiotic, multiple representative MK resistant mutants were sequenced. In all cases the resistant strains contained a point mutation in a MK biosynthesis gene and in almost all cases no other mutations were detected in the genome (FIG. 11). Finally, by ITC an exothermic response was detected when liposomes (1,2-dioleoyl-sn-glycero-3-phosphocholine (DOPC):1,2-dioleoyl-sn-glycero-3-phospho-rac-(1-glycerol(DOPG)=1:1, mol/mol) containing MK were added into each syn-BNP. The calculated dissociation constants between MK and these antibiotics ranged from 0.09-0.30 μM (FIG. 19). In contrast, no syn-BNP induced an exothermic reaction when UQ instead of MK was included in the liposomes (FIG. 20). The interaction between MK and syn-BNP derivatives with cyclization modes that showed reduced antibacterial activity was also tested (FIG. 2). All of these showed higher dissociation constants than to their more active counter parts. For example, the calculated dissociation constant for the fatty acid cyclized version of MBA6, MBA6-cFA, was 45.5 μM (FIG. 21). This was 175-fold lower than the dissociation constant observed for the interaction between MK and the active, side chain cyclized, MBA6. The strong correlation it was observed between dissociation constant and antibiotic potency indicated a direct link between the binding of these antibiotics to MK and cell killing (FIG. 21). While the “GXLXXXW” motif containing natural product lysocin E has been reported to bind lipid II, in ITC experiments with liposomes containing lipid II an antibiotic specific binding between syn-BNP MBAs and lipid II was not observed (FIG. 22 and FIG. 23). Collectively, these data provided multiple distinct lines of evidence that “GXLXXXW” motif containing syn-BNPs specifically bound MK and their antibacterial activity was dependent on MK.

### Anti-Mtb Activity and Mode of Action Analysis for MBAs

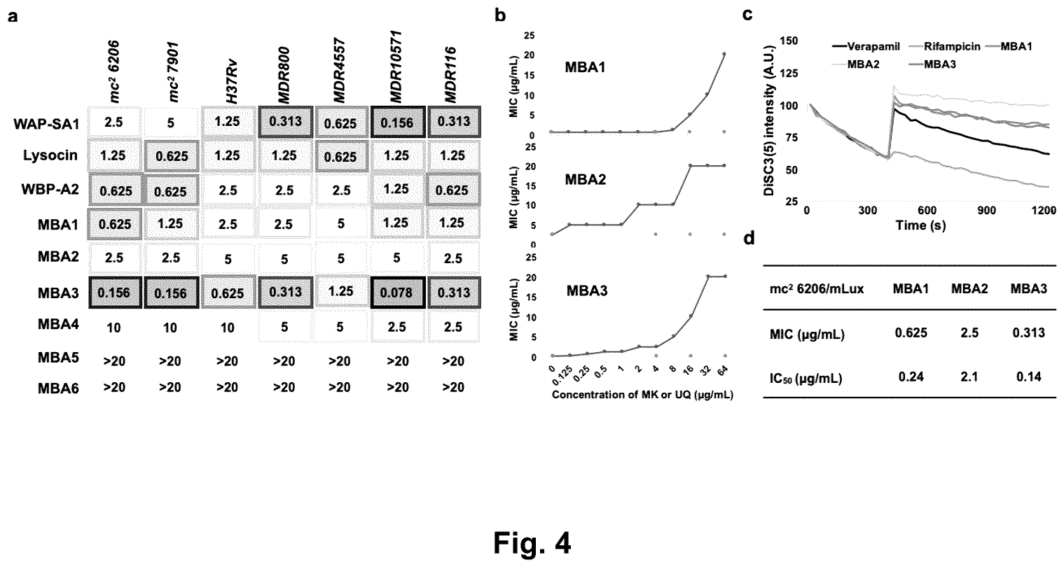

Tuberculosis remains one of the deadliest infectious diseases in the world. Anti-Mtb agents with novel modes-of-action are urgently needed due to the rapid emergence of MDR and extensively drug-resistant Mtb mutants (Saravanan, Microb. Pathog. 117, 237-242 (2018); WHO. Global Tuberculosis Report 2019. World Health Organization (2019)). Although enzymes in the MK biosynthesis pathway have been explored as potential anti-Mtb targets (Libardo, Curr. Opin. Pharmacol. 42, 81-94 (2018); Wellington, ACS Infect. Dis. 4, 696-714 (2018); Berube, Antimicrob. Agents Chemother. 63, e02661-18 (2019)), to the best of the knowledge MK binding has not been tested as a mode of action for an anti-Mtb agent. MBAs 1 through 6 were assayed against a panel of Mtb strains that included wild-type H37Rv, two mutants that can be studied using BSL2 containment (mc² 6206 and mc² 7901), and four MDR strains (800, 4557, 10571 and 116) (FIG. 4a). All MBAs, with the exception of MBA5 and MBA6, were active against this panel of Mtb strains (MIC≤10 μg/mL). MBA3 was the most potent anti-Mtb compound among MBAs, with an MIC as low as 0.078 μg/mL against MDR Mtb.

As this is the first report of an MBA-family antibiotic having anti-Mtb activity, presenet studies sought to confirm the mode of action of the class of antibiotics against Mtb. As seen with S. aureus when MK was added to the assay media the MIC of MBAs against Mtb increased in a dose-dependent manner (FIG. 4b). UQ, which is structurally related and involved in electron transport in mammalian mitochondria had no effect on the anti-Mtb activity of MBAs (FIG. 4b). As seen with S. aureus, MBAs caused an increase in DiSC₃(5) fluorescence indicating that they also induced membrane depolarization in Mtb (FIG. 4c). Taken together, these data indicated that in Mtb MBA's antibiosis activity remains MK dependent membrane disruption, which represents a new mode of action for inhibiting Mtb growth.

Macrophages play a central role in recognizing and destroying invading pathogens. Mtb, however, is capable of replicating and surviving in macrophages (Pieters, Cell Host & Microbe 3, 399-407 (2008))._ENREF_41 In vitro macrophage infection models, which provide a window into the interaction of host and pathogen have proved to be good initial predictors of drug efficacy against Mtb. The present studies examined the anti-Mtb activity of MBAs in a murine macrophage model. In this assay, J774A.1 mouse macrophages infected with Mtb harboring the mLux plasmid were treated with each Mtb active MBA, and then residual bacterial cell viability inside the macrophages was determined by luminescence measurements. The three tested Mtb active MBAs all inhibited Mtb growth in macrophages with IC₅₀ ranging from 0.14 to 2.1 μg/mL (FIG. 4d). As seen in other assays, MBA3 was the most potent MBA in this assay. Considering the indicated correlation between activity in macrophages and potential activity in vivo (Rastogi, Curr. Microbiol. 33, 167-175 (1996)),_ENREF_43 in the future it will be interesting to explore the in vivo anti-Mtb activity of MBA3.

### Two New MBA Structural Families

Although six identified MBAs share a conserved GXLXXXW sequence that is important for MK-binding, they exhibit significant differences in overall peptide sequence as well as different modes of cyclization and anti-microbial potency. To more easily visualize structural relationships among these antibiotics, linear MBA peptide sequences were aligned and a phylogenetic tree was generated (FIG. 5a). This tree contains three distinct clades, one of which is composed of known (lysocin E and WBP-29479A1) and new MBAs (MBA1 and MBA2), while the other two clades only contain MBAs identified in this study (FIG. 5a). MBA1 and MBA2 are closely related to WBP-29479A1 and lysocin E, respectively. They differ from these known structures by only one amino acid building block in the case of MBA1 and three amino acids in the case of MBA2 (FIG. 5b). The four remaining antibiotics identified in this study make up two new MBA structural families. One of which consists of MBA3 and MBA4 (FIG. 5c). Both structures were cyclized between the hydroxyl on (R)-3-hydroxy-octanoic acid and the C-terminal GABA to form undeca-lipodepsipeptide. Beyond the conserved minimal MK binding sequence, these two structures share a distinct L-Pro-5 and a D-Tyr-9. The L-Pro-5 replaces the D-N-Me-aromatic amino acid that appears in all other known or new MBAs between the G and L in the conserved GXLXXXW motif. Proline like N-methylated amino acids can introduce discrete conformations into cyclic peptides (Laufer, J. Pept. Sci. 15, 141-146 (2009)), indicating these two types of amino acids may play similar roles in MBAs. If this observation is included in the definition of a minimal MK binding motif, it would restrict the first X in the motif to being either an N-Methyl-aromatic amino acid (NMeAAA) or proline [G(NMeAAA/P)LXXXW].

The second new family of MBA antibiotics consists of MBA5 and MBA6 (FIG. 5d). In addition to the conserved GXLXXXW motif, these two peptides share a Ser, Ser, Asn, Thr and Phe at positions 1, 3, 8, 9 and 11, respectively. Both peptides are cyclized using the serine at the first position in the linear peptide. However, they differ by the size of the resulting macrocycle. MBA6 contains 12 amino acids, while MBA5 contains 14 amino acids, making it the largest MBA characterized to date. Unlike other MBAs, MBA5 and 6 do not contain any positively charged amino acids. Positively charged residues found in known MBAs as well as other classes of lipopeptide antibiotics have been proposed to interact with the anionic polar head groups of the bacterial membrane and help induce rapid bacteriolysis (Stark, Antimicrob. Agents Chemother. 46, 3585-3590 (2002); Kaji, Chemistry 22, 16912-16919 (2016)). The absence of cationic residues in MBA5 and MBA6 is therefore a key distinguishing feature of this new subclass of MBAs.

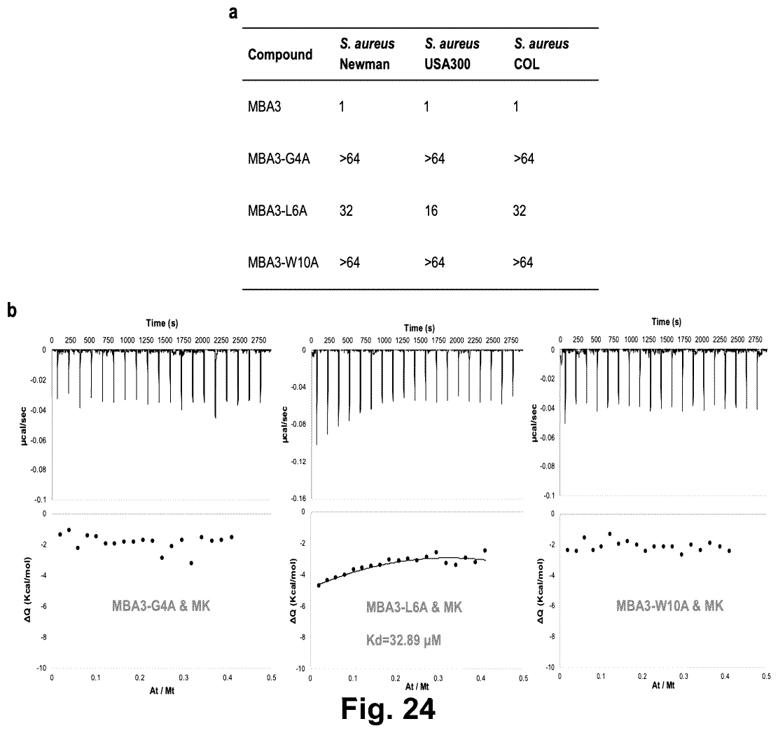

To explore the importance of the individual residues of the proposed minimal MK-binding motif, analogs of the most potent syn-BNP MBA, MBA3 were synthesized, where the three conserved amino acids (Gly-4, L-Leu-6 and D-Trp-10) were individually replaced with L-Ala or D-Ala. The resulting three analogs with one amino acid change each were assayed for both antibacterial activity and MK binding (FIG. 24). Changing L-Leu-6 to L-Ala decreased the antibiotic activity of MBA3 by 16 to 32-fold and its MK binding affinity by 365-fold, while changing Gly-4 to L-Ala or D-Trp-10 to D-Ala completely abrogated its antibacterial activity (MIC>64 μg/mL) as well as its ability to interact with MK. All three conserved residues of the proposed minimal MK-binding motif are therefore critical for both potent antibacterial activity and high affinity MK-binding. How the [G(NMeAAA/P)LXXXW]sequence that is conserved across MBAs interacts with MK remains to be determined. However, it is likely that the indole of Trp interacts with the quinone from MK (Kaupp, Biochemistry 41, 2895-2900 (2002)), and that the N-methyl aromatic amino acid and the proline induce similar cyclic peptide conformations that help create a MK binding pocket. The hydrophobic Gly and Leu residues as well as the hydrophobic lipid tail seen in all MBAs are likely important for interacting with either the hydrophobic polyprenyl tail of MK or the lipid biolayer in bacterial membranes.

### New Sources of MBAs

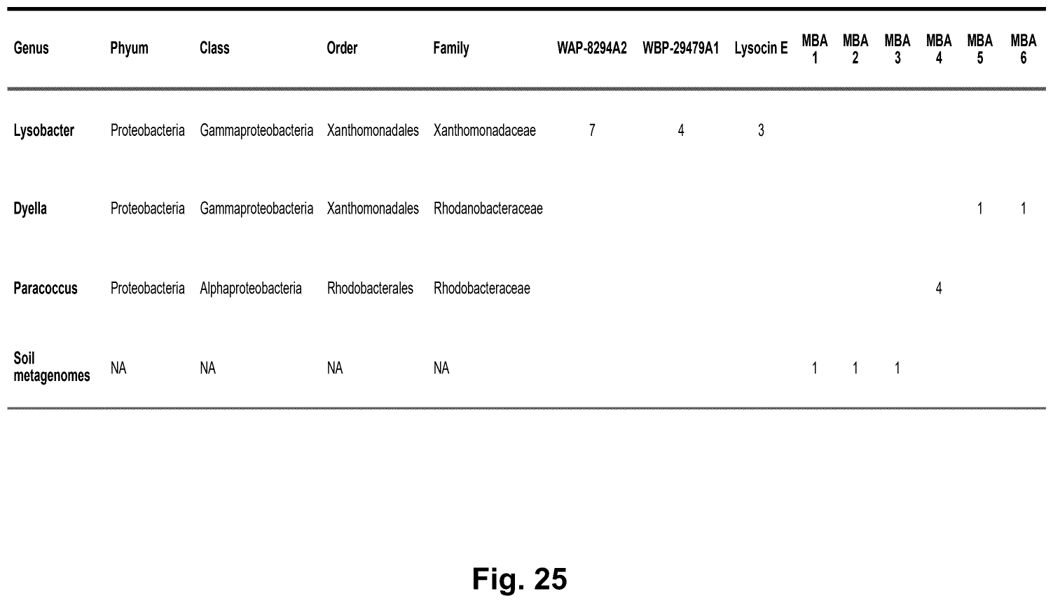

Known MBAs are produced by the genus Lysobacter (Yu, ACS Synth. Biol. 9, 1989-1997 (2020)). Interestingly, both new MBA families were inspired by BGCs that are found in bacteria from different taxa (FIG. 5a and FIG. 25). While the BGC for MBA3 was cloned from a soil metagenome and therefore its source is unknown, the BGC for MBA4 is found in the genome of P. alcaliphilus. BGCs for MBA5 and MBA6 were found in the genomes of D. tabacisoli and D. mobilis, respectively (FIG. 5a and FIG. 25). Paracoccus and Dyella are genera of Proteobacteria that have not traditionally been part of bacterial natural product discovery programs (Masschelein, Nat. Prod. Rep. 34, 712-783 (2017); Liu, Nat. Prod. Rep. 36, 573-592 (2019)). P. alcaliphilus is an alkaliphilic facultative methanol-utilizing bacterium, while the genus Dyella was only first described in 2005 (Urakami, Int. J. Syst. Bacteriol. 39, 116-121 (1989); Xie, Int. J. Syst. Evol. Microbiol. 55, 753-756 (2005)). As outlined in metagenomic library screening studies described above, the L-Leu-6 A-domain is highly conserved across MBA BGCs. In fact, when eDNA A-domain NPSTs were compared to known MBA BGC L-Leu-6 A-domain, all NPSTs that returned e-values ≤10⁻⁴⁵ were found to arise from an MBA BGC (FIG. 12). To more extensively explore MBA BGC diversity in the environment, an archived collection of A-domain NPSTs generated from diverse soil metagenomes for MBA BGC-like L-Leu-6 A-domain sequences was screened.²² One in every 25 screened soils (80 out of 2,000) contained a unique MBAL-Leu-6-like A-domain NPST (i.e., e-value ≤10⁻⁴⁵). The studies therefore not only identified additional cultured sources of structurally diverse MBAs, but also indicated that there is a large number of as yet uncharacterized MBA BGCs in soil microbiomes. ENREF_50

### Activity of MBA3 and MBA6 in a Murine Peritonitis-Sepsis Model

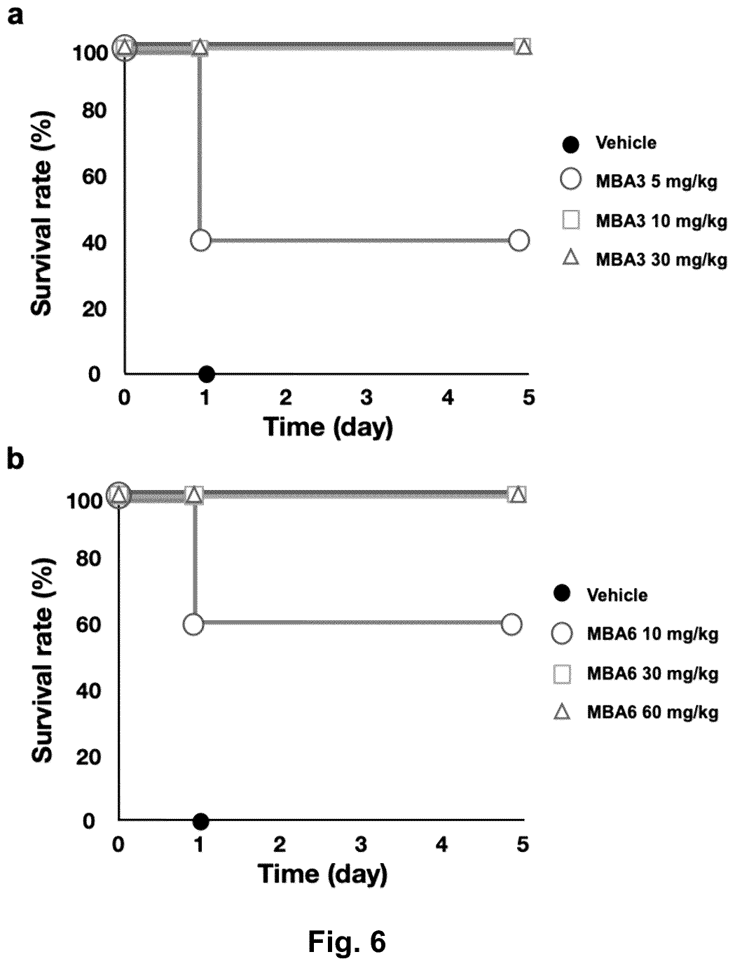

Using a mouse peritonitis-sepsis model the studies examined the in vivo efficacy of MBA3 and MBA6, which were the most potent analogs of the two new MBA families identified in this study. As shown in FIG. 6, treatment of a methicillin-resistant S. aureus COL infection with either MBA3 (5, 10 and 30 mg/kg) or MBA6 (10, 30 and 60 mg/kg) dramatically decreased the mortality of infected mice compared to treatment with vehicle alone (30% solutol). A minimal dose of 10 and 30 mg/kg for MBA3 and MBA6, respectively, was required for 100% survival. Collectively, the results indicated that MBA3 and MBA6 could be valuable candidates for the development of therapeutics for treating MDR S. aureus infections.

### DISCUSSION

In this study sequence based metagenomic mining were combined with a pattern search of bioinformatically generated natural product structures to identify BGCs that would encode MBAs. Synthesis of the structures bioinformatically predicted to arise from these BGCs produced six MK-binding cyclic lipodepsipeptides with diverse structures, anti-Mtb activity and potent in vivo anti-S. aureus activity. Although MBAs exhibit significant differences in peptide sequence and different cyclization modes, they share a conserved GXLXXXW sequence that is a minimal MK-binding motif. Notably, the most potent members of the two new MBA structural families identified, MBA3 (metameb) and MBA6 (mobimeb), proved effective at treating methicillin-resistant S. aureus infections in a murine peritonitis-sepsis model. The discovery of new MBA structural families indicated that MBAs are a diverse and still underexplored class of naturally occurring antibiotics. Both the new MBA structures reported here, as well as the MBA search tools developed in this study, should prove useful in ultimately identifying a member of this mechanistically interesting class of antibiotics that can be successfully brought through therapeutic development into clinical use.

MBA resistance can arise from mutations in MK or heme biosynthesis. In both cases, these mutants show a small colony variant (SCV) phenotype (Proctor, Nat. Rev. Microbiol. 4, 295-305 (2006)). In addition, both MK and heme deficient mutants have been found to show reduced virulence in animal models Proctor, Nat. Rev. Microbiol. 4, 295-305 (2006)). When growth-compensatory mutants were directly selected using a menB point mutant background all growth-compensatory mutants showed increased MK production (Lannergird, Antimicrob. Agents Chemother. 52, 4017-4022 (2008)). When growth-compensatory mutants were selected by serial passage using either MK-deficient or heme-deficient strains ˜96% of the mutants showed restored MK levels (Cao, mBio 8, e00358-17 (2017)). These studies indicated that while growth-compensatory mutations will undoubtedly arise, most of the mutants are likely to restore susceptibility to MBAs. When the mutation frequency is taken together with decreased virulence by these SCVs and the restoration of MK production in the majority of growth-compensatory mutants that MBAs are likely to show a lower clinically relevant resistance rate than indicated by the resistance frequency seen in the laboratory. With that said, this will clearly be something that will need to be explored in more detail in the clinical development process.

Although studies have previously used metagenomic mining methods to identify BGCs of interest, this is the first case of identifying molecules with a desired mode of action by searching for a substructure among a large dataset of bioinformatically predicted natural products. The success of this approach for identifying novel classes of MBAs indicated that bioinformatic structure prediction algorithms have developed to the extent that direct structure, or sub-structure, searches of collections of bioinformatically predicted natural product structures now represents an alternative generalizable approach to screen sequenced BGCs for the potential production of bioactive small molecules.

In summary, the emergence of multidrug-resistant bacteria poses a threat to global health and necessitates the development of additional in vivo active antibiotics with diverse modes of action (MOA). Directly targeting menaquinone (MK), which plays an important role in bacterial electron transport, is an appealing, yet underexplored, MOA due to a dearth of MK-binding molecules. Here sequence-based metagenomic mining were combined with a motif search of bioinformatically predicted natural product structures to identify six biosynthetic gene clusters that encode MK-binding antibiotics (MBAs). Their predicted products (MBA1-6) were rapidly accessed using a synthetic-bioinformatic natural product (syn-BNP) approach, which relies on bioinformatic structure prediction followed by chemical synthesis. Among these six structurally diverse MBAs, four makeup two new MBA structural families. The most potent member of each new family (MBA3, MBA6) proved effective at treating methicillin-resistant Staphylococcus aureus infection in a murine peritonitis-sepsis model. The only conserved feature present in all MBAs is the sequence “GXLXXXW”, which represents a minimum MK-binding motif Notably, a subset of MBAs was found to be active against Mycobacterium tuberculosis both in vitro and in macrophages. The findings indicated that naturally occurring MBAs are a structurally diverse and untapped class of mechanistically interesting, in vivo active, antibiotics.

### Methods

### Chemical Reagents, Consumables and Instruments.

All reagents were purchased from commercial sources and used without further purification. Pre-loaded 2-chlorotrityl resin for peptide synthesis was purchased from Matrix Innovation, Inc (Quebec, Canada). Reagents for solid-phase peptide synthesis (SPPS), DCM (dichloromethane), DIPEA (N,N-diisopropylethylamine), DMAP (4-dimethylaminopyridine), DMF (dimethylformamide), HATU (0-(7-azabenzotriazol-1-yl)-N,N,N′,N″-tetramethyluronium hexafluorophosphate), HFIP (hexafluoroisopropanol), PyAOP ((7-azabenzotriazol-1-yloxy) tripyrrolidinophosphonium hexafluorophosphate) and TFA (trifluoroacetic acid), were purchased from P3 BioSystems (Louisville, KY). Standard N-Fmoc amino acid building blocks were purchased from P3 BioSystems and Chem-Impex International (Wood Dale, IL). (R)-3-hydroxy-octanic acid and Fmoc-N-Me-D-Phe-OH were purchased from Enamine (Monmouth, NJ). Fmoc-N-Me-D-Trp(Boc)-OH was purchased from Alabiochem (Suzhou, China). Fmoc-N-Me-D-Tyr(tBu)-OH was purchased from 1 ClickChemistry (Kendall Park, NJ). Fmoc-GABA-OH was purchased from Sigma-Aldrich (St. Louis, Missouri). All solvents used for chromatography were HPLC grade or higher. MTT (thiazolyl blue tetrazolium bromide) and Type II mucin from porcine stomach were purchased from Sigma-Aldrich. Fluorescent dyes SYTOX Green and DISC₃(5) (3,3′-dipropylthiadicarbocyanine Iodide) were purchased from ThermoFisher Scientific (Waltham, MA), and the assay results were recorded using a Tecan Infinite M Nano⁺ plate reader (Morrisville, pNC). 1,2-dioleoyl-sn-glycero-3-phosphocholine (DOPC) and 1,2-dioleoyl-sn-glycero-3-phospho-rac-(1-glycerol) (DOPG) were purchased from Avanti Polar Lipids (Alabaster, Alabama). Menaquinone-4 (MK4) and ubiquinone-10 (UQ10) were purchased from Sigma-Aldrich and Gram-positive Lys-lipid II was purchased from Antimicrobial Discovery Solutions Ltd (Coventry, UK).

For all liquid chromatography, solvent A=H₂O (0.1% v/v formic acid) and solvent B=CH₃CN (0.1% v/v formic acid). UPLC-LRMS data were acquired on a Waters Acquity system equipped with QDa and PDA detectors, a Phenomenex Synergi Fusion-RP 80 Å column (2.0×50 mm, 4 μm) and controlled by Waters MassLynx software. The following chromatographic conditions were used for UPLC-LRMS: 5% B from 0.0 to 0.9 min, 5% to 95% B from 0.9 to 4.5 min, 95% B from 4.5 to 5.0 min, 95% to 5% B from 5.0 to 5.4 min, and 5% B from 5.4 to 6 min (flow rate of 0.6 mL/min and 10 μL injection volume). HPLC-HRMS data were acquired on a SCIEX ExionLC HPLC coupled to an X500R QTOF mass spectrometer, equipped with a Phenomonex Kinetex PS C18 100 Å column (2.1×50 mm, 2.6 μm) and controlled by SCIEXOS software. The following chromatographic conditions were used for UPLC-HRMS: 5% B from 0.0 to 1.0 min, 5% to 95% B from 1.0 to 10.0 min, 95% B from 10.0 to 12.5 min, 95% to 5% B from 12.5 to 13.5 min, and 5% B from 13.5 to 17.0 min (flow rate of 0.4 mL/min and 1 μL injection volume). Peptide purification was performed using an Agilent 1200 Series HPLC with UV detection and equipped with an XBridge Prep C18 130 Å column (10×150 mm, 5 μm). ¹H and ¹³C NMR spectra were acquired on a Bruker Avance DMX 600 MHz spectrometer equipped with cryogenic probes (The Rockefeller University, New York). All spectra were recorded at 25 or 50° C. in DMSO-d₆. Chemical shift values are reported in ppm and referenced to residual solvent signals: 2.50 ppm (¹H) and 39.52 ppm (¹³C).

### Identification of Metagenomic MBA BGCs.

Previously archived soil eDNA cosmid libraries were probed to recover BGCs predicted to encode novel MBAs. Construction, PCR screening with barcoded A-domain degenerate primers, amplicon sequencing and read processing for these cosmid libraries have been described in detail previously. Using A-domain sequences from the three known MBAs as references, the eSNaPD (environmental Surveyor of Natural Product Diversity) software package was used to identify similar sequences among A-domain amplicon sequences generated from archived metagenomic libraries. The library well locations of hits found in this analysis were identified using the barcode parsing functionality of the eSNaPD software package. Clones associated with select eDNA A-domain hits were then recovered from the appropriate library wells using a previously described dilution PCR strategy. Recovered cosmids were sequenced using a MiSeq Reagent Nano Kit v2 on a MiSeq sequencer (Illumina) and the resulting reads were assembled into contigs using Newbler 2.6 (Roche). Assembled complete and partial BGCs were analyzed using antiSMASH v5.1.2 and the in-house NRP predictor to predict the substrate specificity of each A-domain. When using the in-house NRP predictor, building blocks were predicted by comparing the Stachelhaus code of predicted A-domain to that of A-domains from known NRPs.

Screening ˜2000 Soils to Explore More Potential MBAs. eDNA was extracted from ˜2000 ecologically and geographically diverse soil samples and Natural Product Sequence Tags (NPSTs) of soil metagenomes were generated using a previously established pipeline. These NPSTs were then searched using the eSNaPD pipeline against the manually curated L-Leu-6 sequences from the three known and six new MBA BGCs. A-domain amplicons that matched MBA L-Leu-6 at an e-value ≤10⁴⁵ were considered as hits. A multiple sequence alignment of all qualifying hit sequences was generated using MUSCLE, and the resulting alignment file was used to generate a maximum likelihood tree with FastTree.
Construction of an in-House Predicted Non-Ribosomal Peptide (p-NRP) Database.

GenBank files for 38,933 NRP BGCs representing 10,858 complete bacterial genome assemblies were retrieved from the antiSMASH-db. The p-NRP database was constructed from the BGCs by synthesizing data from five A-domain prediction resources: antiSMASH-db (Blin, Nucleic Acids Res. 49, D639-D643 (2021)), the NORINE amino acid database (Flissi, Nucleic Acids Res. 48, D465-D469 (2020)), A-domain substrate predictions from MIBiG (Kautsar, Nucleic Acids Res. 48, D454-D458 (2020)), SANDPUMA (Chevrette, Bioinformatics 33, 3202-3210 (2017)) and the own NRP BGC analyses (FIG. 14). All BGCs were analyzed using antiSMASH v5.1.2 and the parallel job execution tool GNU Parallel (Samdani, Comput. Biol. Chem. 74, 39-48 (2018)). After removing duplicate entriesa total of 36,957 NRPS antiSMASH v5 regions was obtained. The entirety of the “monomer” dataset was downloaded from NORINE, which represents known A-domain substrates as well as other known amino acids, and it was used as the basis for the own A-domain “substrates” table. This table was extended with the addition of auxiliary columns, such as the substrate's charge as well as normalizing the shorthand names of some substrates to follow convention more closely in the NRP BGC community. Lastly, the studies incorporated predictions for Stachelhaus codes using MIBiG, which stores curated information about well-characterized BGCs, and SANDPUMA which maintains a table of known A-domain-to-substrate specificities. A-domain substrate specificity codes obtained from the manual curation of characterized NRPs were also included. For any substrates not represented in the NORINE database, a search was conducted in the PubChem database and the relevant data was imported (Kim, Nucleic Acids Res. 49, D1388-D1395 (2021)), including the PubChem ID to ensure uniqueness. Substrates which did not appear in PubChem were still included in the database; however, other uniquely identifying information, such as IUPAC name and SMILES string, were relied upon to prevent a substance being erroneously represented multiple times. When no substrate specificity code was identified for an A-domain, the A-domain substrate specificity was predicted using the NRPSPredictor2 “small cluster prediction” (Rottig, Nucleic Acids Res. 39, W362-W367 (2011)). Finally, based on this collection of curated substrate specificity codes, a table of linear peptide sequence predictions for each NPRS BGC was generated. The starting point of a predicted NRP sequence was determined either by the presence of a condensation starter (Cs) domain, or the presence of A-domain with no immediately preceding condensation (C) domain. The end of the peptide sequence was defined by the presence of a thioesterase (TE) domain. Ultimately for each BGC the order of the monomers predicted in the A-domain analysis followed the order of A-domains predicted by antiSMASH for that BGC. To identify potential MBA BGCs the resulting p-NRP database was searched for the full GXLXXXW motif as well as two partial motifs: GXL and LXXXW.

Peptide Synthesis. Solid Phase Peptide Synthesis (SPPS):

All peptides were synthesized using standard Fmoc-based solid-phase peptide synthesis methods on 2-chlorotrityl chloride resin using commercially available Fmoc-protected amino acids. Peptide synthesis started from the conserved Leu seen at 6th position of each peptide. Pre-loaded Leu on 2-cholorotrityl resin (0.3 g, 0.552 mmol/g) was swollen in DCM for 20 min at room temperature then drained and washed with DMF (3 mL, 3×). Coupling of individual amino acids was carried out by using Fmoc-protected amino acids (2 equiv., relative to resin loading) mixed with HATU (2 equiv.) and DIPEA (2 equiv.) in DMF (5 mL). Coupling reactions were carried out for 1 h with occasional swirling then washed with DMF (3 mL, 3×). Fmoc-deprotection was done using 20% piperidine in DMF (3 mL) for 7 min and repeated twice. The resin was washed with DMF (3 mL, 5×) and then coupled with a subsequent amino acid.

### Ester Bond Formation:

Ester bonds were formed either between the hydroxyl group on the N-terminal fatty acid or an amino acid associated hydroxyl group and the C-terminal carboxyl group of the peptide. To carry out ester bond formation, the resin was mixed with amino acid (20 equiv.), DIPEA (40 equiv.), benzoyl chloride (20 equiv.) and DMAP (0.8 equiv.) in 10 mL DCM and gently shaken for 72 h. After the ester bond formation, remaining amino acids were coupled as described above.

### Peptide Cyclization:

Peptides were cleaved from the resin by treatment with 20% HFIP in DCM for 2 h. After air drying overnight the cleaved linear peptide was cyclized without purification using PyAOP (8 equiv.) and DIPEA (30 equiv.) in DMF (50 mL). After 2 h, DCM (100 mL) was added and washed repeatedly with 1% formic acid in water (5 mL, 10×). The extracted cyclic peptide was air dried overnight.

### Final Cleavage:

Air dried cyclic peptide was dissolved in 3 mL cleavage cocktail (95% (v/v) TFA, 2.5% (v/v) triisopropylsilane and 2.5% (v/v) water) for 1.5 h. A cold mixture of diethyl ether:hexane (1:1) was then added and kept in −20° C. for 10 min to precipitate the peptide. Peptide pellets were harvested by centrifuging (2500×g) for 5 min, re-dissolved in 5 mL methanol and dried in vacuo overnight.

### Peptide Purification by HPLC.

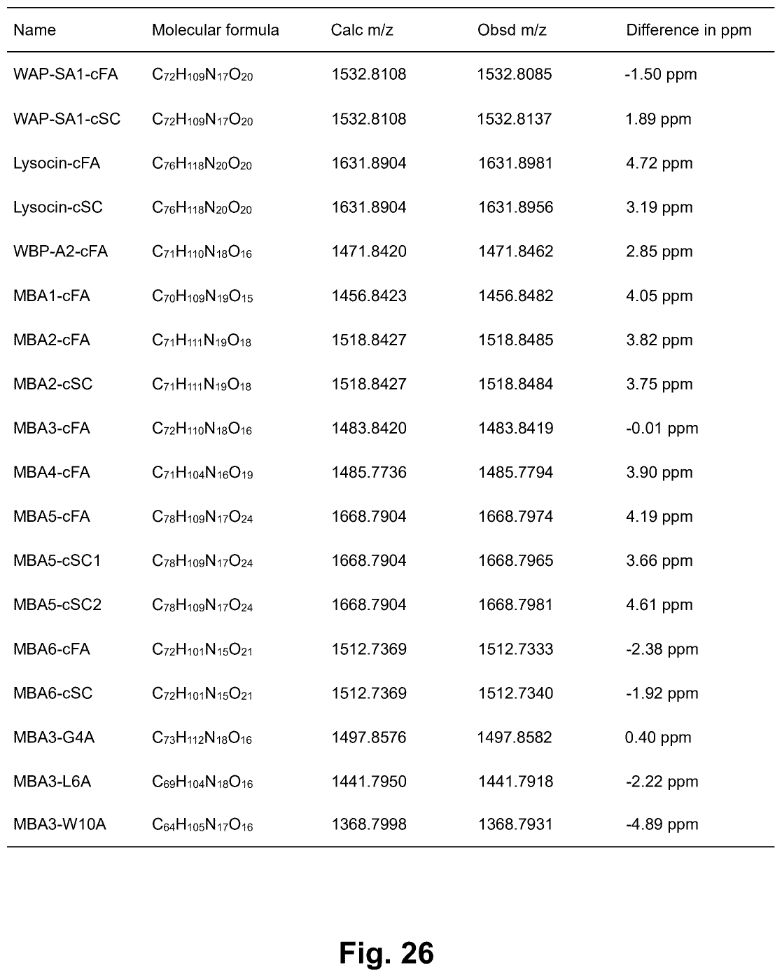

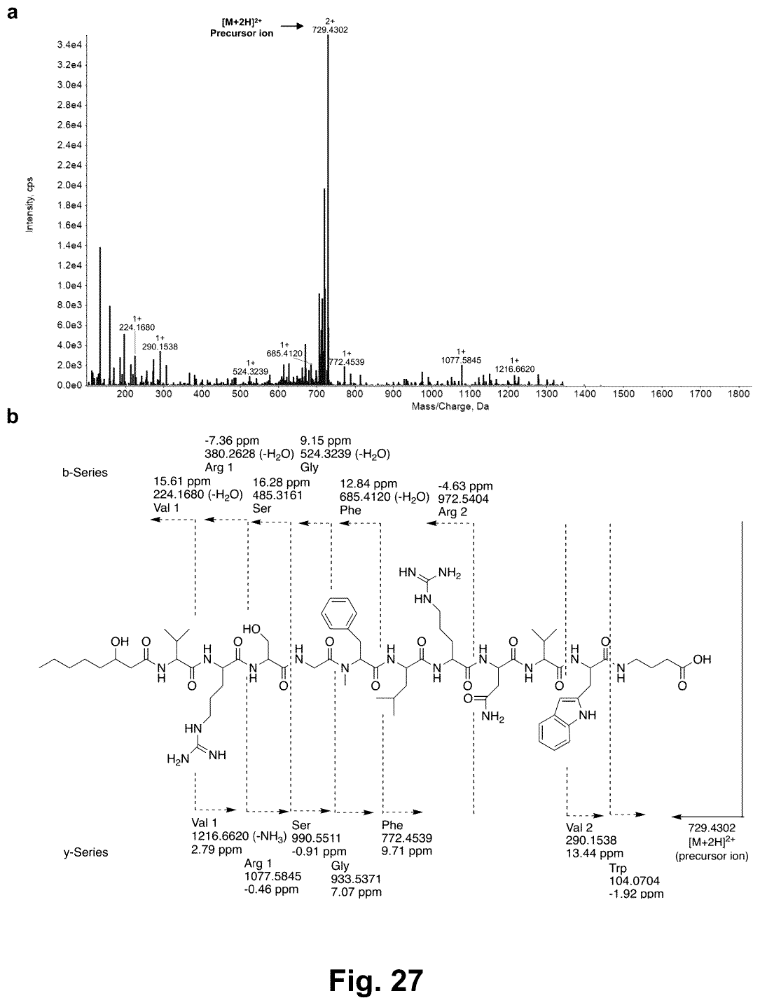

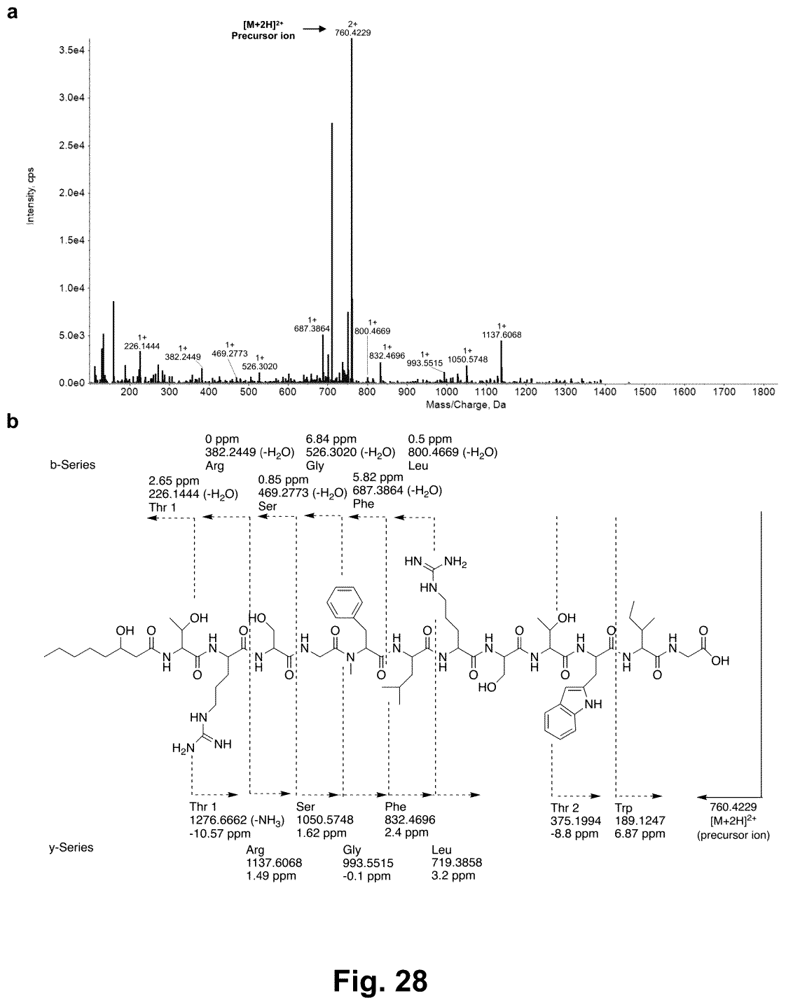

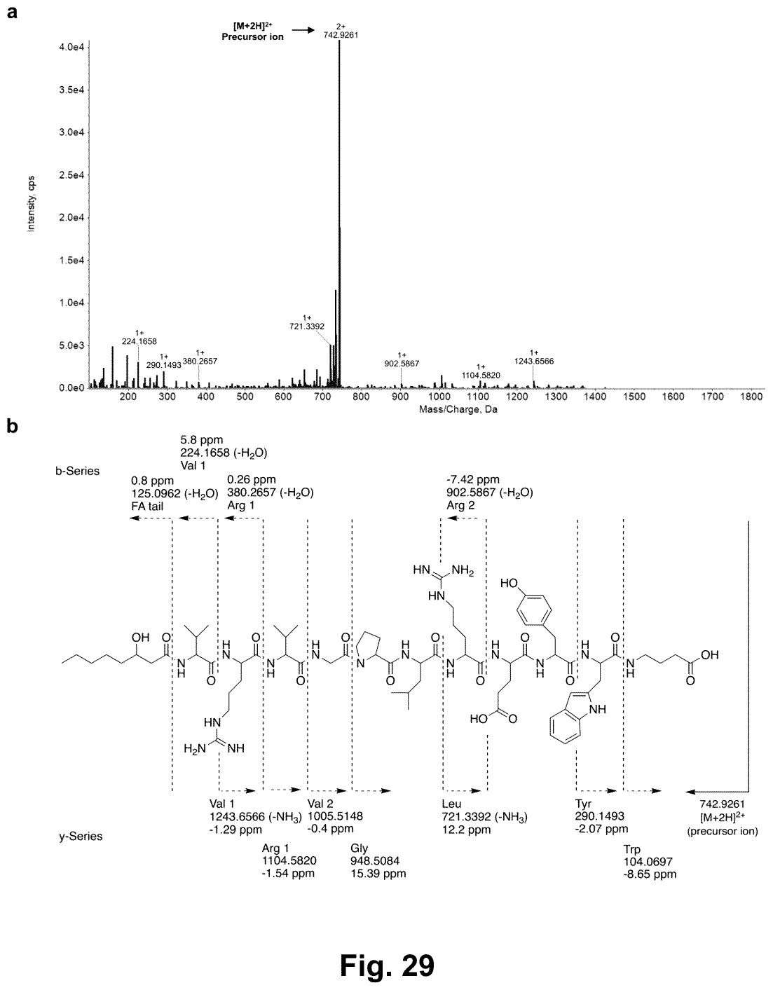

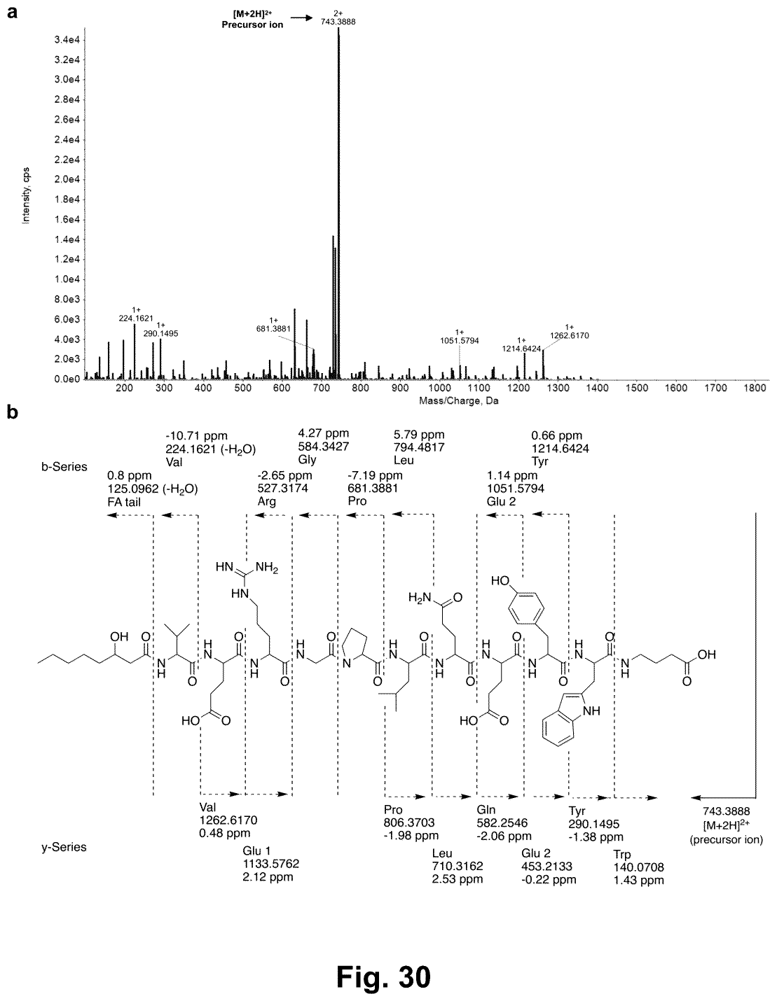

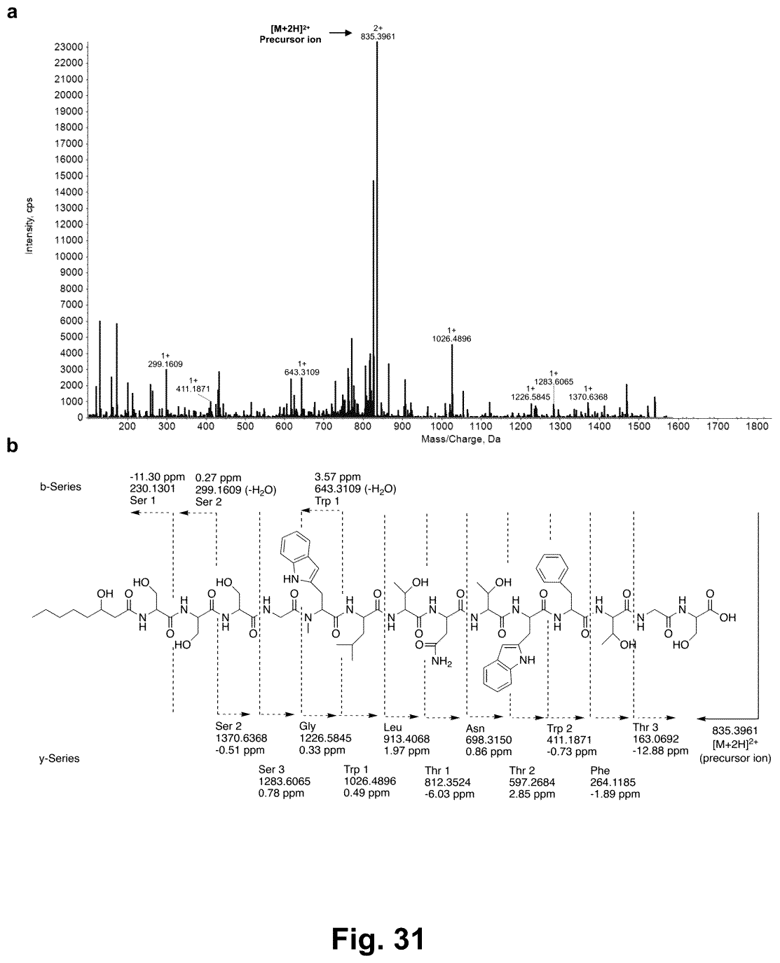

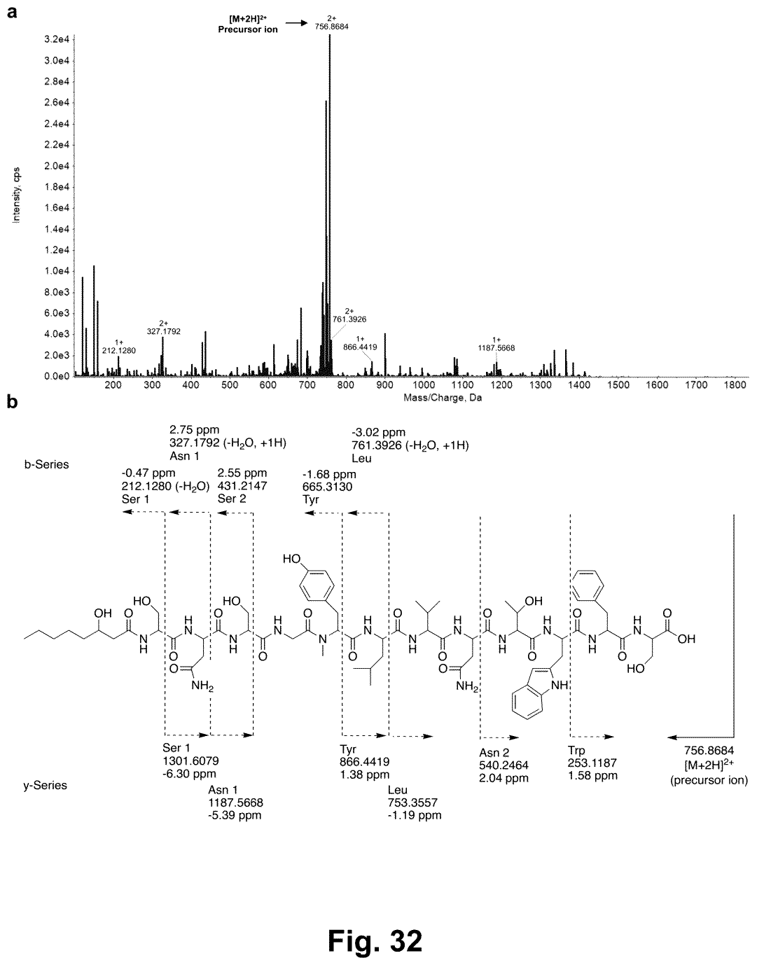

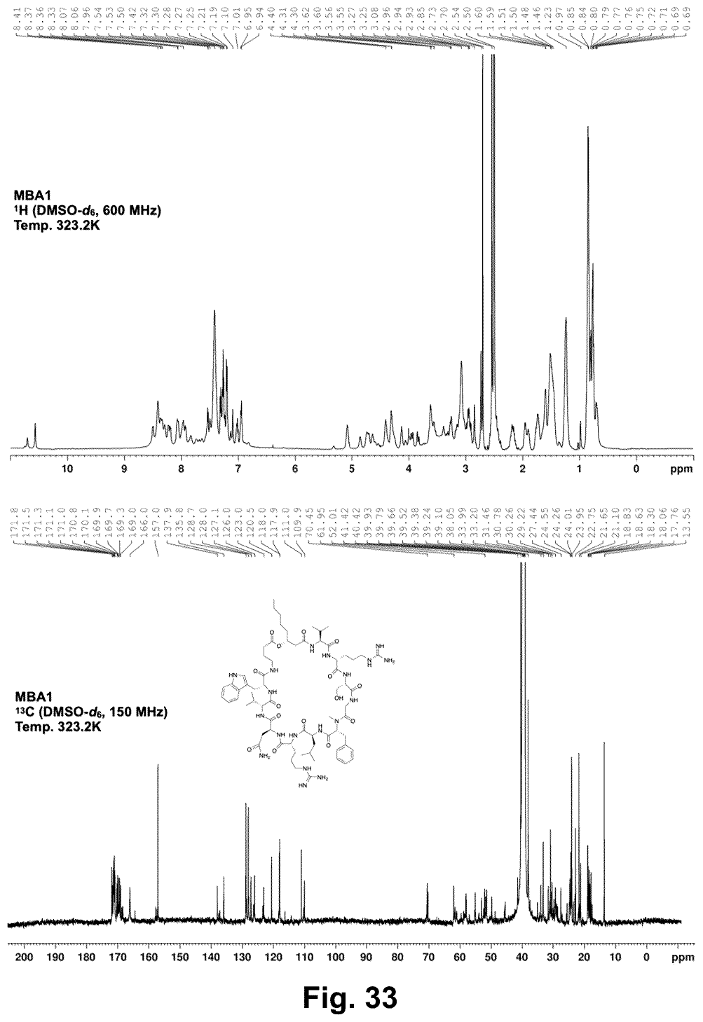

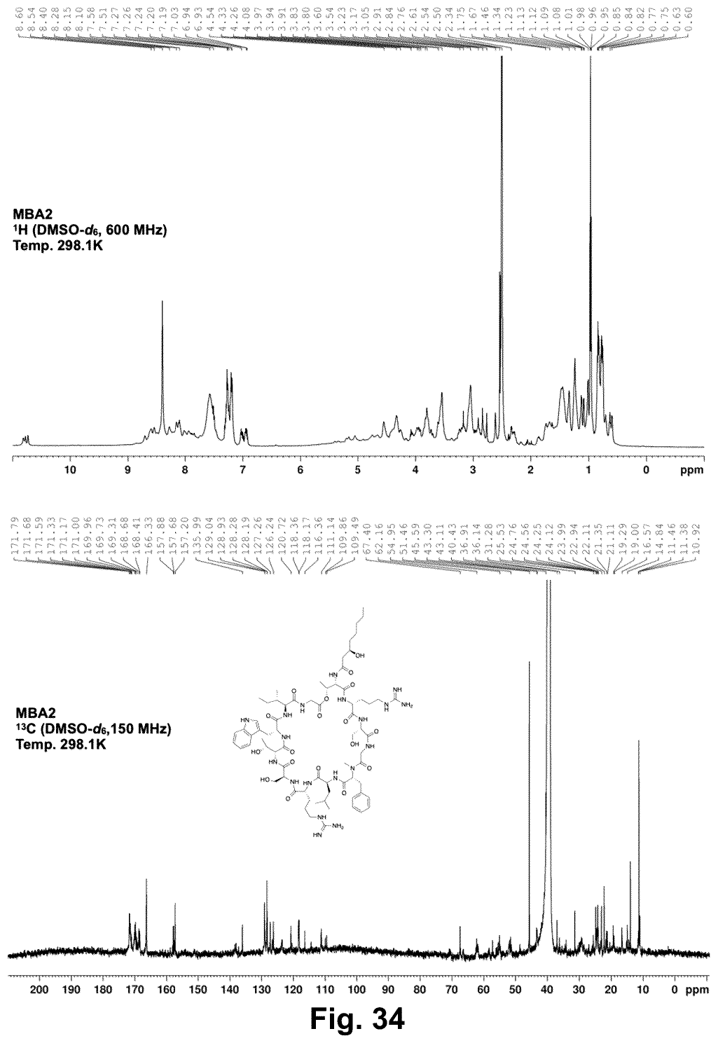

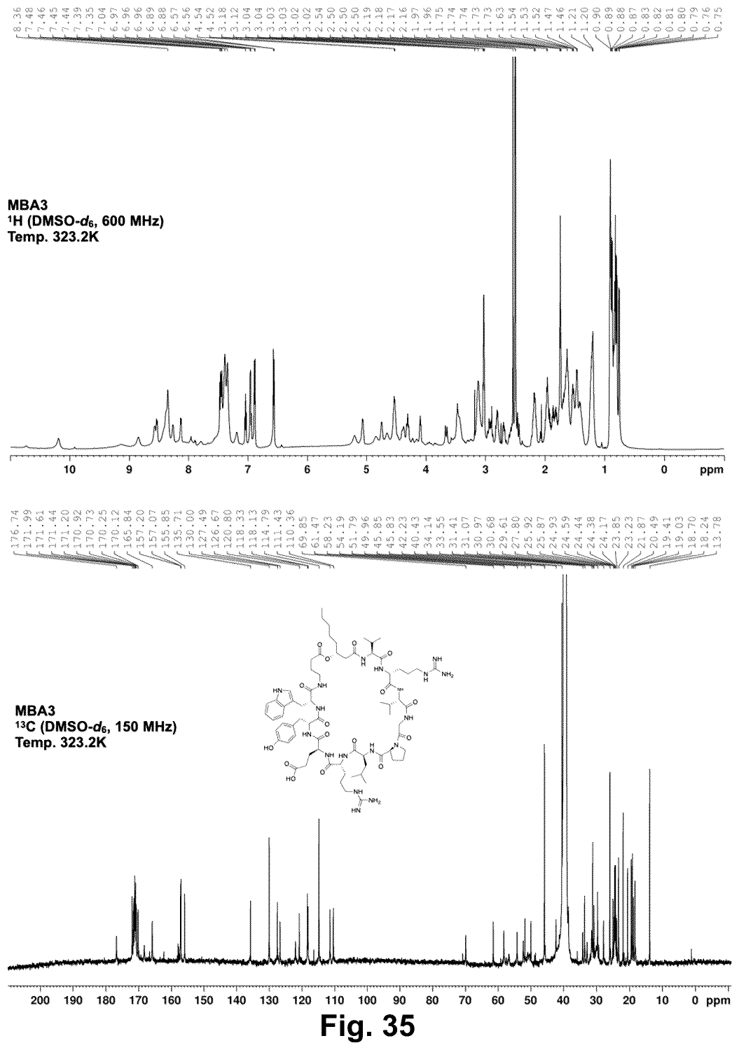

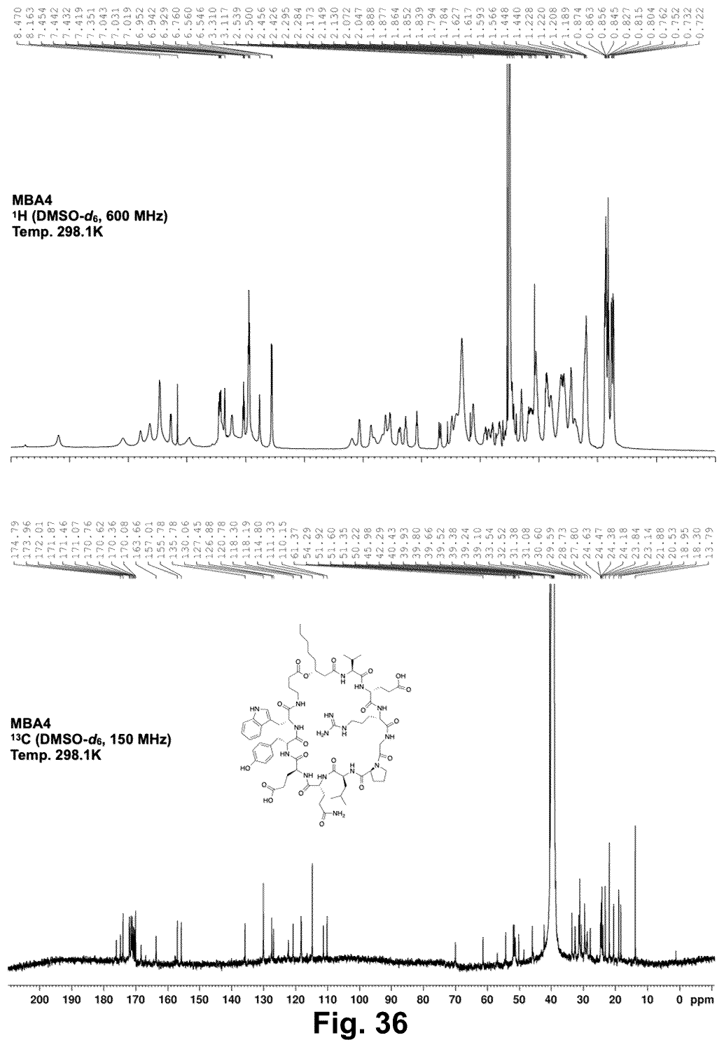

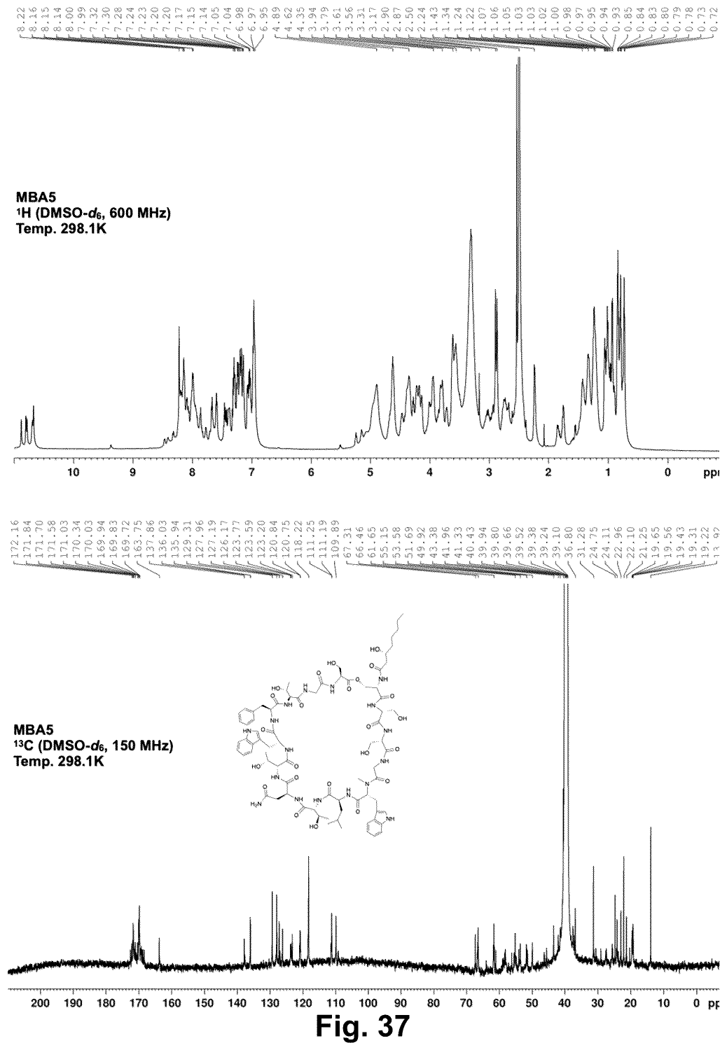

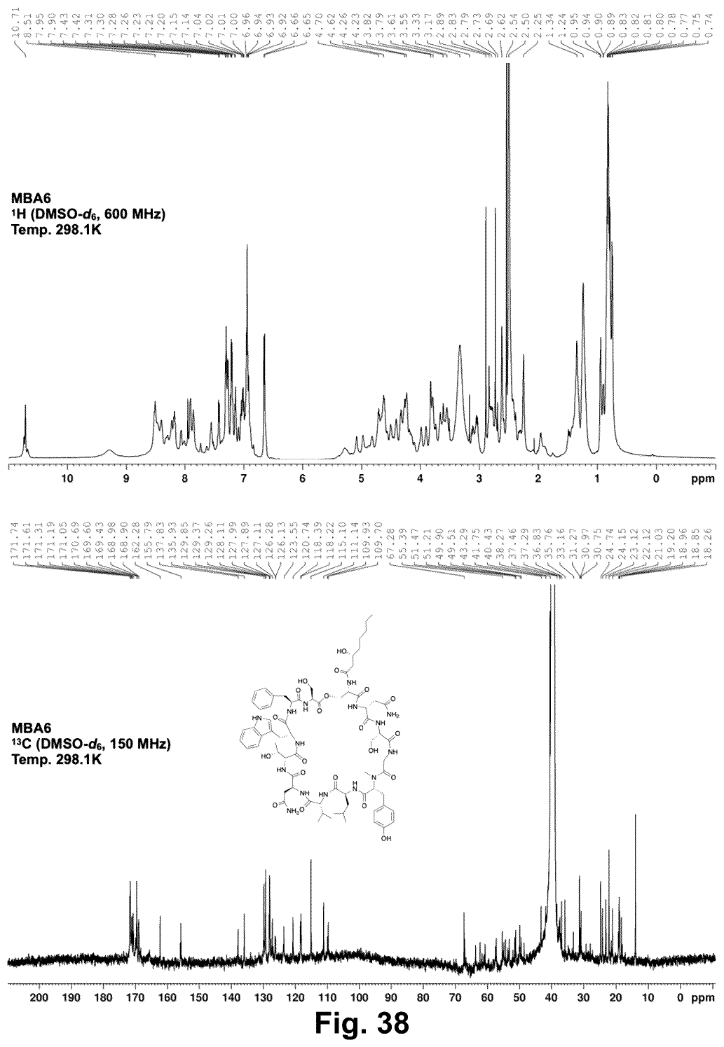

Crude cyclic lipopeptides were purified on a Xbridge Prep C18 HPLC column using a dual solvent system (A/B: water/acetonitrile, supplemented with 0.1% (v/v) formic acid). All peptides were eluted using a linear gradient from 30 to 50% gradient of B. Peptide purity was confirmed by UPLC. The identity of each peptide was confirmed by HRMS (FIG. 26) and tandem MS (FIG. 27 through FIG. 32) analyses. ¹H NMR and ¹³C NMR spectra were recorded for each peptide (FIG. 33 through FIG. 38). Pure peptides were dissolved in DMSO at 6.4 mg/mL for MIC measurements and mode of action studies.

### Antimicrobial Assays Against Gram-Positive Bacteria, Gram-Negative Bacteria and Yeast Pathogens.

All antimicrobial assays were run in 96-well microtiter plates using a broth micro-dilution method. Diluted overnight cultures were used in all assays. For yeast strains, overnight cultures were diluted 2000-fold in YPD broth. For Enterococcus faecium and Staphylococcus aureus, overnight cultures were diluted 1000- and 10,000-fold in LB broth, respectively. For Streptococcus strains, overnight cultures were diluted 5,000-fold in Brain Heart Infusion (BHI) broth. For other bacteria, overnight cultures were diluted 5,000-fold in LB broth. 100 μL of each diluted culture was mixed with 100 μL of LB broth containing a syn-BNP at 2-fold serial dilutions across a 96-well microtiter plate row. The final concentration of each compound ranged from 64 to 0.25 μg/mL. Plates were incubated at 37° C. (bacteria) or 30° C. (yeast) for 16 h. The lowest concentration that inhibited visible microbial growth was recorded as the minimum inhibition concentration (MIC). All MIC assays were done in duplicate (n=2).

Antibacterial Assay Against Mycobacterium smegmatis mc² 155.

M. smegmatis mc² 155 was shaken (200 rpm) in 7H9 broth (supplemented with 0.2% glucose, 0.2% glycerol and 0.05% tyloxapol) for 48 h at 37° C. The culture was then diluted to an OD₆₀₀ of 0.005, and 100 μL was added to 100 μL of 7H9 broth containing each syn-BNP at 2-fold serial dilutions across a 96-well plate row. The final concentration of each compound ranged from 64 to 0.25 μg/mL. Plates were incubated for 48 h at 37° C. and then 30 μL of Alamar Blue cell viability reagent (Thermo Fisher Scientific) was added. After an additional 24 h incubation, the wells that remained blue by visual inspection were deemed to contain inhibitory concentrations of each antibiotic. All MIC assays were done in duplicate (n=2).

Antibacterial Assay Against Mycobacterium tuberculosis.

Mtb BSL2 mc² 6206, BSL2 mc² 7901, wild-type H37Rv and four multidrug-resistant strains (116, 800, 4557 and 10571) were passaged in 7H9 broth (supplemented with oleic acid-albumin-dextrose-catalase, 0.2% glycerol and 0.02% tyloxapol) at 37° C. to OD₆₀₀ of 0.5-0.7. The culture was then diluted to an OD₆₀₀ of 0.005, and 100 μL of the diluted culture was distributed in 96-well plates. 100 μL of 7H9 broth containing each syn-BNP at 2-fold serial dilutions across a 96-well plate row was added to give final test concentrations ranging from 20 to 0.039 μg/mL. The plates were then incubated at 37° C. and 5% CO₂. After incubation for 6 days, 30 μL of Alamar Blue cell viability reagent was added, the cultures were incubated for another 24 h, and the absorbance was read at 570 nm and normalized to 600 nM. All MIC assays were done in duplicate (n=2).

### Murine Macrophage Infection.

The activity of each MBA against intracellular Mtb was determined by infecting J774A.1 mouse macrophages with Mtb mc² 6206 harboring the mLux plasmid (Mtb mc² 6206/mLux). Macrophages were initially suspended at a concentration of 4-5×10⁵ cells/mL in Dulbecco's Modified Eagle Medium (DMEM, Sigma-Aldrich) supplemented with Fetal Bovine Serum (FBS, 10%) and L-glutamate (2 mM). Flat bottom 96-well white plates were seeded with 100 μL of the macrophage suspension and incubated overnight to allow cells to adhere to the plates. Mtb mc2 6206/mLux was grown to mid-log phase (OD₆₀₀=0.5-0.7). Mtb cultures were then spun down, washed with Phosphate-Buffered Saline (PBS), and resuspended in DMEM containing 10% FBS, L-glutamate (2 mM), pantothenic acid (50 μg/mL) and leucine (50 μg/mL). The assay plates were inoculated with 100 μL of Mtb mc² 6206/mLux at a multiplicity of infection of 1:10 and incubated for 4 h to allow Mtb to infect the macrophages. The adhered macrophages were then washed twice with 100 μL PBS and then 100 μL of DMEM was added to each well. After a 1 h incubation plates were washed twice with 100 μL of PBS. 100 μL of each MBA serial diluted in DMEM (from 64 to 0.0004 μg/mL) was added to the assay plates. Plates were then incubated at 37° C. for 72 h. Residual Mtb cell viability inside macrophages was determined by luminescence measurement on a Spark multimode microplate reader (Tecan). Dose response curves were generated by non-linear regression in GraphPad Prism v8 and plotted as the logarithm of concentration vs normalized percent cell viability. The antibiotic concentration that led to 50% cell viability (IC₅₀) was determined from the dose-response curves. Each treatment was carried out in triplicate (n=3).

### Membrane Lysis Assay.

Membrane lysis assays were done in 384-well black microtiter plates. An overnight culture of S. aureus USA300 was collected by centrifugation and resuspended in PBS to give an OD₆₀₀ of 0.5. SYTOX Green (5 mM, 1 μL) was added to the cell suspension (2.5 mL), which was then incubated in the dark at room temperature for 10 min. Fluorescence intensity of the mixture was recorded continually at 2 s intervals (Ex/Em 488/523 nm). When the signal stabilized the appropriate amount of each antibiotic (6.4 mg/mL DMSO stock solutions) to give 2× its MIC was added and immediately mixed by manual pipetting. Vancomycin and lysocin were used as negative and positive controls, respectively. Data were presented as the relative intensity with respect to the average fluorescence signal prior to the addition of the MBA. All assays were done in duplicate (n=2). A representative fluorescence recording for each antibiotic is shown in FIG. 3b.

### Membrane Depolarization Assay.

Membrane lysis assays were done in 384-well black microtiter plates. An overnight culture of S. aureus USA300 was collected by centrifugation and resuspended in PBS to give an OD₆₀₀ of 0.5. 100 μL of this cell suspension and 20 μM DiSC₃(5) (50 μL) were added to 300 μL of PBS, and then incubated in the dark at room temperature for 15 min. KCl (2 M, 50 μL) was then added and incubated for another 15 min. Fluorescence intensity of the mixture was recorded continually at 2 s intervals (Ex/Em 643/675 nm). When the signal stabilized the appropriate amount of each MBA (6.4 mg/mL DMSO stock solutions) to give 2× its MIC was added and immediately mixed by manual pipetting. Vancomycin and lysocin were used as negative and positive controls, respectively. Data were presented as the relative intensity with respect to the average fluorescence signal prior to the addition of the MBA. All assays were done in duplicate (n=2). A representative fluorescence recording for each antibiotic is shown in FIG. 18a.

### Antibiotic Resistant Mutant Selection.

A single S. aureus USA300 colony was inoculated into LB medium and grown overnight at 37° C. (200 rpm). A portion of the overnight culture containing approximately 10⁹ cells was diluted ( 1/10× or 1/40×) into LB containing each antibiotic at 4× its MIC. The resulting mixtures were distributed into microtiter plates at 5 μL per well. After incubating statically at 37° C. overnight, colonies that appeared were transferred into fresh LB agar plates. The MICs of 4-8 individual colonies were then determined using the microtiter dilution method described above. DNA was extracted from cultures of colonies that showed an elevated MIC relative to the wild-type, and the resulting DNA was sequenced using MiSeq Reagent Kit V3 (MS-102-3003, Illumina). Single-nucleotide polymorphisms (SNPs) for each new MBA were identified using SNIPPY (http://github.com/tseemann/snippy) by mapping MiSeq reads to the reference genome of S. aureus USA300_FPR3757 (RefSeq assembly accession: GCF_000013465.1) (FIG. 18b).

### Isothermal Titration Calorimetry (ITC).

For MK and UQ binding, a 1:1 mixture of 1,2-dioleoyl-sn-glycero-3-phosphocholine (DOPC) and 1,2-dioleoyl-sn-glycero-3-phospho-rac-(1-glycerol) (DOPG) containing either 1.25 mol % MK4 or UQ10 was dissolved in chloroform. A lipid film was generated by drying this material under a stream of nitrogen followed by 2 h of vacuum drying. The resulting film was hydrated using 10 mM HEPES (pH 7.5) with 100 mM NaCl to give a final total lipid concentration of 5 mM. Using an Avanti Mini Extruder, this suspension was passed through a 100 nm polycarbonate filter 10 times. For ITC the sample cell was filled with 400 μL of 25 μM MBA prepared in 10 mM HEPES buffer. The syringe (150 μL) was loaded with a 5 mM lipid suspension with 1.25 mol % MK4 or UQ10. For lipid II binding, 10 mM DOPC containing 1 mol % lipid II was dissolved in chloroform. The resulting film was hydrated using 50 mM Tris (pH 7.5) with 100 mM NaCl and passed through a 100 nm polycarbonate filter 10 times. For ITC the sample cell was filled with 400 μL of 25 μM an MBA prepared in 50 mM Tris buffer. The syringe (150 μL) was loaded with a 10 mM DOPC suspension with 1 mol % lipid II. Data were collected by using an Auto-iTC200 (Malvern Panalytical) and processed by Affinimeter software using the “one binding site” model.

MK Extraction and Identification in S. aureus.

MK extraction was performed using a previously reported lysozyme-chloroform-methanol extraction method (Xie, BMC Microbiol. 21, 175 (2021)). Cultures of the menA deletion mutant (ΔmenA), the hemB transposon insertion mutant (tn:hemB), S. aureus Newman and USA300 were grown overnight in LB liquid media. Cultures of the menA and hemB mutants were adjusted to the same OD₆₀₀ as the S. aureus Newman and USA300 cultures. Cells from 20 mL of each density normalized culture were collected by centrifugation. The resulting cell pellets were suspended in 50 mL of 10 mM Tris-HCl buffer (pH 7.4) containing 5 mg of lysozyme and then incubated at 37° C. for 1 h. This mixture was then centrifuged for 10 min at 2,500×g to collect the lysozyme-treated cells. 10 mL of chloroform/methanol (2:1, v/v) was added to the cell pellets to extract MK. This extraction process was repeated three times. The chloroform/methanol extracts were combined and evaporated under vacuum. The dried material was suspended in 50 μL of chloroform/methanol (2:1, v/v) for analysis by thin layer chromatography (TLC). MK extracts were spotted on TLC LuxPlate silica gel 60 F254 (Millipore) plates and the plates were developed in a mixture of hexane and diethyl ether (85:15, v/v). MK was visualized by UV exposure, and the plates were photographed. Finally, MK bands were collected from the TLC plates and eluted using isopropanol. Isopropanol-eluted MK was analyzed by HPLC-IRMS and MK4 was used as a standard.

### Mtb Membrane Depolarization Assay.

Mtb membrane depolarization assays were done in a 384-well black microtiter plate. Mtb mc2 6206 was grown to exponential phase (OD₆₀₀=0.5-0.7), washed twice with HG buffer (5 mM HEPES and 5 mM glucose, pH 7.2) and resuspended in HG buffer (OD₆₀₀=0.1). DiSC₃(5) (4 μM) was added to the cell suspension and incubated in the dark at room temperature for 2 h. Fluorescence intensity of the mixture was recorded continually at 2 s intervals (Ex/Em 622/670 nm). After the fluorescence intensity stabilized each antibiotic (6.4 mg/mL DMSO stock) was added to give a final concertation of 2× its Mtb MIC and immediately mixed by manual pipetting. Rifampicin and verapamil were used as the negative and positive controls, respectively. Data were presented as the relative intensity with respect to the average fluorescence signal prior to the addition of each MBA. All assays were done in duplicate (n=2). A representative fluorescence recording for each antibiotic is shown in FIG. 4c.

### Cytotoxicity Assessment.

HEK293 human cells were grown at 37° C. in a 5% CO₂ atmosphere in Dulbecco's modified Eagle medium (DMEM) supplemented with fetal bovine serum (10% v/v), L-glutamate (2 mM), penicillin (10 units/mL) and streptomycin (10 units/mL). HEK293 cells were seeded into 96-well flat bottom microtiter plates (target density of 2500 cells per well) and incubated in DMEM at 37° C. for 24 h to allow cells to adhere. The DMEM medium was then removed by aspiration and replaced with 100 μL of fresh DMEM medium containing each antibiotic at 10 serially diluted concentrations ranging from 32 to 0.0625 ug/mL. After 48 h at 37° C. the DMEM media was removed and 100 μL of a MTT solution (10 uL of 5 mg/mL MTT in PBS premixed with 100 μL of DMEM) was added into each well. After 4 h at 37° C. the solution was aspirated from each well. Precipitated formazan crystals were dissolved by addition of 100 μL of solubilization solution (40% DMF, 16% SDS and 2% glacial acetic acid in H₂O). OD₅₇₀ readings were used to calculate relative growth (%) based on the positive (2 μM Taxol) and negative (DMSO) controls. Dose response curves were generated by non-linear regression in GraphPad Prism v8. Cytotoxicity assays were performed in triplicate (n=3).

### Mouse Peritonitis-Sepsis Model.

Female outbred Swiss Webster mice were used in all experiments. S. aureus COL was grown in Mueller-Hinton Broth at 37° C. overnight and diluted with 7% type II porcine stomach mucin supplemented with 0.2 mM FeNH₄-citrate. Cultures were diluted to provide a challenge inoculum of ˜5×10⁸ CFU in 0.2 mL. 0.2 mL of the challenge inoculum was administered via intraperitoneal injection. 35 mice were randomly grouped into five per cohort and each cohort was given a single dose of either vehicle (30% solutol), MBA3 at 5, 10 or 30 mg/kg or MBA6 at 10, 30 or 60 mg/kg 1 h after infection via subcutaneous injection. Mice were maintained in accordance with American Association for Accreditation of Laboratory Care criteria. The Rockefeller University Animal Care and Use Committee approved all animal procedures.

Binding to MK Rather than Lipid II is the Primary Mode of Action of Syn-BNP MBAs

Mutations in heme biosynthesis have been reported to provide resistance to the “GXLXXXW” motif containing natural product lysocin E. In that study it was postulated that heme deficiency provides resistance because lysocin E's ultimate mode of action is binding to lipid II, which is almost undetectable when heme biosynthesis is disrupted. ITC has been used to validate the binding between lipid II and a number of antibiotics, including gallidermin, nisin, vancomycin and teixobactin. To test whether syn-BNP MBAs also interacted with lipid II ITC was performed using lipid II containing liposomes and two representative pairs of active and inactive syn-BNP structures (i.e., active/inactive: MBA3/MBA3-W10 Å or MBA6/MBA6-cFA). While the nisin positive control showed a high lipid II binding affinity with a Kd of 0.07 μM, the active and inactive pairs of syn-BNP structures either showed no lipid II binding or similarly low lipid II binding affinities, indicating that there was not a specific interaction between antibiotics in this family and lipid II (FIG. 22). In addition to the ITC analysis, each syn-BNP MBA were also tested for antibacterial activity against a hemB transposon insertion mutant (tn:hemB) and found that this strain was resistant to all six syn-BNP MBAs (MIC>64 μg/mL) (FIG. 9). Interestingly, the four known lipid II binders that were tested as controls (lysobactin, nisin, ramoplanin and vancomycin) showed no significant differences in MIC between wild-type S. aureus and the tn:hemB strain (FIG. 9). Based on the differences in lipid II binding affinity and activity against the tn:hemB strain between known lipid II binders and syn-BNP MBAs studies sought an explanation, other than lipid II binding, that could explain why the tn:hemB strain was resistant to MBAs. As MK deficiency is known to confer resistance to MBAs, studies looked at the level of MK in the tn:hemB strain. Interestingly, it was found that MK was undetectable by high-resolution mass spectrometry (HRMS) in this strain (FIG. 23), which likely explains this strain's resistance to syn-BNP MBAs. This observation mimics the finding that syn-BNP MBAs were active against native MK-producing strains, but were inactive against all MK-deficient Enterococcus and Strepococcus strains that were tested (FIG. 9) (Johnston et al., 2020, Curr. Opin. Struct. Biol. 65, 33-41; Boersch et al., 2018, RSC Adv. 8, 5099-5105). In contrast, all four known lipid II binders that were tested were active against all eight Enterococcus or Streptococcus strains (FIG. 9). When taken together these observations support an interaction with MK rather than lipid II as the primary mode of action of the syn-BNP antibiotics identified in this study.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound comprising the amino acid sequence (X^{A})ₐG(X^{B})_{b}L(X^{C})_{c}W(X^{D})_{d},
wherein each occurrence of X^{A}, X^{B}, X^{C}, and X^{D} is independently selected from the group consisting of a natural amino acid, functionalized natural amino acid, unnatural amino acid, functionalized unnatural amino acid, and any combination thereof; and
wherein each occurrence of a, b, c, and d is independently an integer from 0 to 100.

2. The compound of claim 1, wherein each occurrence of a, b, c, and d is independently an integer from 0 to 10.

3. The compound of claim 1, wherein the compound is a cyclic compound.

4. The compound of claim 1, wherein the amino acid sequence (X^{A})ₐG(X^{B})_{b}L(X^{C})_{c}W(X^{D})_{d} comprises at least one amino acid sequence selected from the group consisting of at least one amino acid sequence, or a fragment thereof, selected from FIG. 1; at least one amino acid sequence, or a fragment thereof, selected FIG. 5; at least one amino acid sequence, or a fragment thereof, selected from FIG. 7; at least one amino acid sequence, or a fragment thereof, selected from FIG. 12; at least one amino acid sequence, or a fragment thereof, selected from FIG. 13; at least one amino acid sequence, or a fragment thereof, selected from FIG. 15; at least one amino acid sequence, or a fragment thereof, selected from FIG. 16; the amino acid sequence GXLXXXW; and any combination thereof,
wherein each occurrence of X is independently selected from the group consisting of a natural amino acid, functionalized natural amino acid, unnatural amino acid, functionalized unnatural amino acid, and any combination thereof.

5. The compound of claim 1, wherein the compound is a compound having the structure of 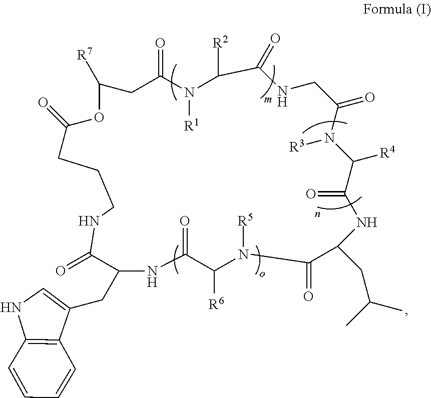
or a racemate, an enantiomer, a diastereomer, a pharmaceutically acceptable salt, or a derivative thereof, or 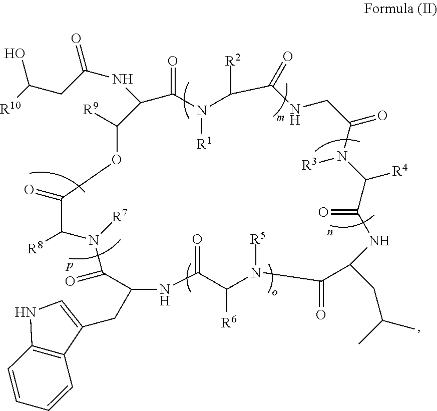
or a racemate, an enantiomer, a diastereomer, a pharmaceutically acceptable salt, or a derivative thereof,
wherein each occurrence of R¹, R², R³, R⁴, R⁵, R⁶, R⁷, R⁸, R⁹, and R¹⁰ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
wherein R¹ and R² are optionally fused or joined to form a ring;
R³ and R⁴ are optionally fused or joined to form a ring;
R⁵ and R⁶ are optionally fused or joined to form a ring; and
R⁷ and R⁸ are optionally fused or joined to form a ring; and
wherein each occurrence of m, n, o, and p is independently an integer from 0 to 100.

6. The compound of claim 5, wherein the compound having the structure of Formula (I) is a compound having the structure of Formula (Ia) 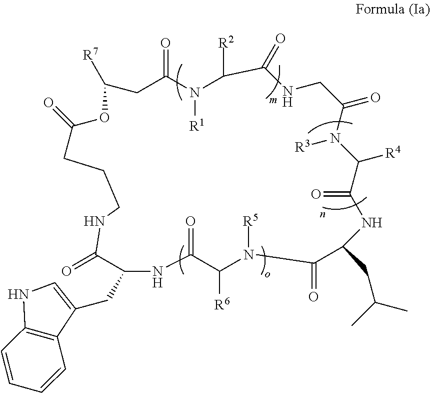
or a racemate, an enantiomer, a diastereomer, a pharmaceutically acceptable salt, or a derivative thereof,
wherein each occurrence of R¹, R², R³, R⁴, R⁵, R⁶, R⁷, R⁸, R⁹, and R¹⁰ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
wherein R¹ and R² are optionally fused or joined to form a ring;
R³ and R⁴ are optionally fused or joined to form a ring; and
R⁵ and R⁶ are optionally fused or joined to form a ring; and
wherein each occurrence of m, n, and o is independently an integer from 0 to 100.

7. The compound of claim 5, wherein the compound having the structure of Formula (II) is a compound having the structure of Formula (IIa) 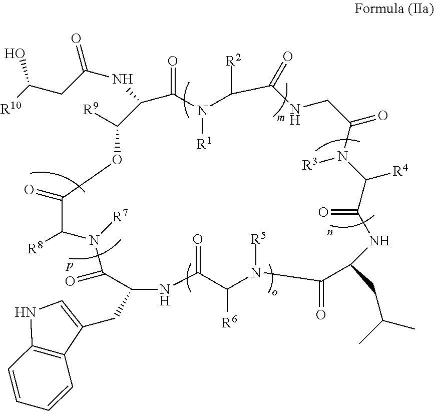
or a racemate, an enantiomer, a diastereomer, a pharmaceutically acceptable salt, or a derivative thereof,
wherein each occurrence of R¹, R², R³, R⁴, R⁵, R⁶, R⁷, R⁸, R⁹, and R¹⁰ is independently selected from the group consisting of hydrogen, deuterium, halogen, alkyl, cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy, carboxyl, carboxylate, ester, ═O, —NO₂, —CN, sulfoxy, sulfonyl, alkyl sulfonyl, secondary amide, tertiary amide, an amino acid, and any combinations thereof;
wherein R¹ and R² are optionally fused or joined to form a ring;
R³ and R⁴ are optionally fused or joined to form a ring;
R⁵ and R⁶ are optionally fused or joined to form a ring; and
R⁷ and R⁸ are optionally fused or joined to form a ring; and
wherein each occurrence of m, n, o, and p is independently an integer from 0 to 100.

8. The compound of claim 1, wherein the compound is a compound selected from the group consisting of: 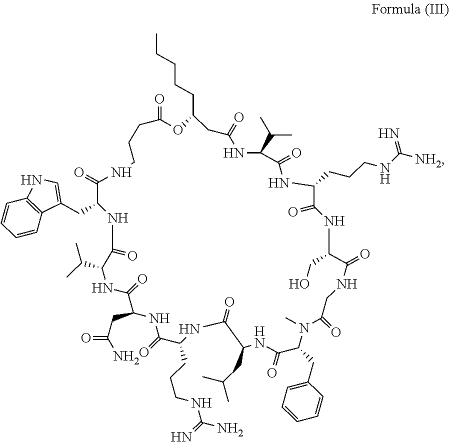 [SMILES: CCCCC[C@@H]1CC(=O)N[C@@H](C(C)C)C(=O)N[C@H](CCCNC(=N)N)C(=O)N[C@@H](CO)C(=O)NCC(=O)N(C)[C@H](Cc2ccccc2)C(=O)N[C@@H](CC(C)C)C(=O)N[C@H](CCCNC(=N)N)C(=O)N[C@@H](CC(N)=O)C(=O)N[C@H](C(C)C)C(=O)N[C@H](Cc2c[nH]c3ccccc23)C(=O)NCCCC(=O)O1]
or a racemate, an enantiomer, a diastereomer, a pharmaceutically acceptable salt, or a derivative thereof, 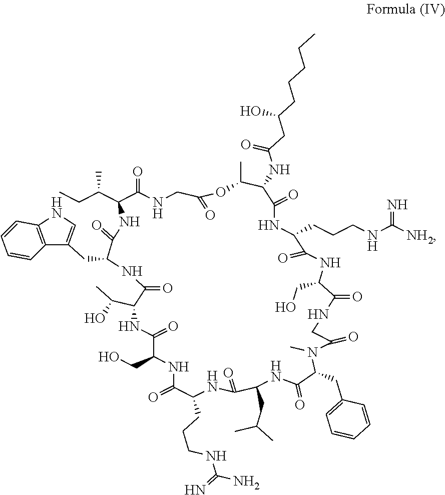 [SMILES: CCCCC[C@@H](O)CC(=O)N[C@@H]1C(=O)N[C@H](CCCNC(=N)N)C(=O)N[C@@H](CO)C(=O)NCC(=O)N(C)[C@H](Cc2ccccc2)C(=O)N[C@@H](CC(C)C)C(=O)N[C@H](CCCNC(=N)N)C(=O)N[C@@H](CO)C(=O)N[C@H]([C@@H](C)O)C(=O)N[C@H](Cc2c[nH]c3ccccc23)C(=O)N[C@@H]([C@@H](C)CC)C(=O)NCC(=O)O[C@@H]1C]
or a racemate, an enantiomer, a diastereomer, a pharmaceutically acceptable salt, or a derivative thereof, 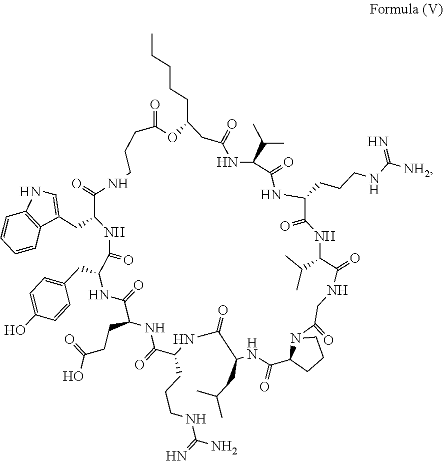 [SMILES: CCCCC[C@@H]1CC(=O)N[C@@H](C(C)C)C(=O)N[C@H](CCCNC(=N)N)C(=O)N[C@@H](C(C)C)C(=O)NCC(=O)N2CCC[C@H]2C(=O)N[C@@H](CC(C)C)C(=O)N[C@H](CCCNC(=N)N)C(=O)N[C@@H](CCC(=O)O)C(=O)N[C@H](Cc2ccc(O)cc2)C(=O)N[C@H](Cc2c[nH]c3ccccc23)C(=O)NCCCC(=O)O1]
or a racemate, an enantiomer, a diastereomer, a pharmaceutically acceptable salt, or a derivative thereof, 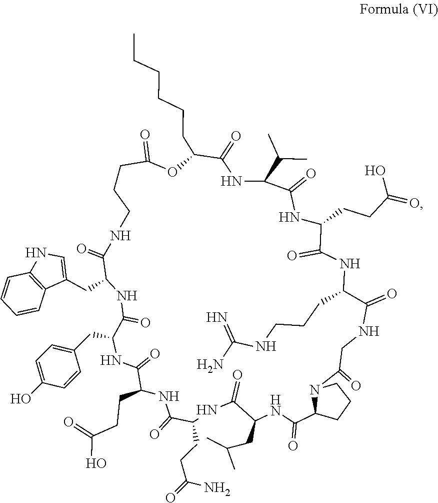 [SMILES: CCCCCC[C@H]1OC(=O)CCCNC(=O)[C@@H](Cc2c[nH]c3ccccc23)NC(=O)[C@@H](Cc2ccc(O)cc2)NC(=O)[C@H](CCC(=O)O)NC(=O)[C@@H](CCC(N)=O)NC(=O)[C@H](CC(C)C)NC(=O)[C@@H]2CCCN2C(=O)CNC(=O)[C@H](CCCNC(=N)N)NC(=O)[C@@H](CCC(=O)O)NC(=O)[C@H](C(C)C)NC1=O]
or a racemate, an enantiomer, a diastereomer, a pharmaceutically acceptable salt, or a derivative thereof, 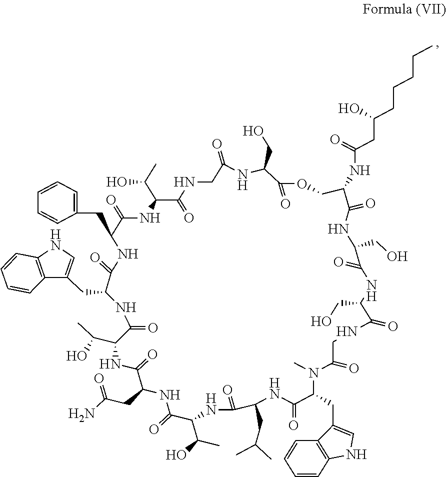
or a racemate, an enantiomer, a diastereomer, a pharmaceutically acceptable salt, or a derivative thereof, and 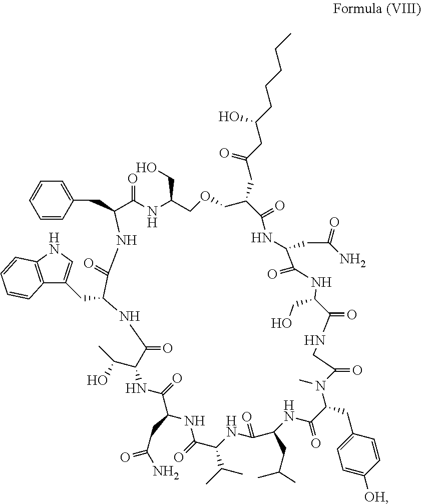
or a racemate, an enantiomer, a diastereomer, a pharmaceutically acceptable salt, or a derivative thereof.

9. The compound of claim 1, wherein the compound specifically binds to menaquinone.

10. A pharmaceutical composition comprising at least one compound of claim 1.

11. An isolated nucleic acid encoding at least one compound of claim 1 or a racemate, an enantiomer, a diastereomer, a pharmaceutically acceptable salt, or a derivative thereof.

12. A genetically engineered cell, wherein the cell encodes at least one compound of claim 1 or a racemate, an enantiomer, a diastereomer, a pharmaceutically acceptable salt, or a derivative thereof.

13. A genetically engineered cell, wherein the cell comprises at least one compound of claim 1 or a racemate, an enantiomer, a diastereomer, a pharmaceutically acceptable salt, or a derivative thereof.

14. An isolated nucleic acid encoding the amino acid sequence (X^{A})ₐG(X^{B})_{b}L(X^{C})_{c}W(X^{D})_{d} or a fragment thereof,
wherein each occurrence of X^{A}, X^{B}, X^{C}, and X^{D} is independently selected from the group consisting of a natural amino acid, functionalized natural amino acid, unnatural amino acid, functionalized unnatural amino acid, and any combination thereof; and
wherein each occurrence of a, b, c, and d is independently an integer from 0 to 100.

15. A genetically engineered cell, wherein the cell encodes at least one amino acid sequence (X^{A})ₐG(X^{B})_{b}L(X^{C})_{c}W(X^{D})_{d} or a fragment thereof,
wherein each occurrence of X^{A}, X^{B}, X^{C}, and X^{D} is independently selected from the group consisting of a natural amino acid, functionalized natural amino acid, unnatural amino acid, functionalized unnatural amino acid, and any combination thereof; and
wherein each occurrence of a, b, c, and d is independently an integer from 0 to 100.

16. A genetically engineered cell, wherein the cell comprises at least one amino acid sequence (X^{A})ₐG(X^{B})_{b}L(X^{C})_{c}W(X^{D})_{d} or a fragment thereof,
wherein each occurrence of X^{A}, X^{B}, X^{C}, and X^{D} is independently selected from the group consisting of a natural amino acid, functionalized natural amino acid, unnatural amino acid, functionalized unnatural amino acid, and any combination thereof; and
wherein each occurrence of a, b, c, and d is independently an integer from 0 to 100.

17. A method of treating or preventing a bacterial infection in a subject in need thereof, wherein the method comprises administering at least one compound of claim 1 or a composition thereof to the subject.

18. The method of claim 17, wherein the subject is exposed to or infected with a pathogen.

19. The method of claim 18, wherein the pathogen is bacteria.

20. The method of claim 19, wherein the bacteria is selected from the group consisting of drug resistant bacteria, gram positive bacteria, and any combination thereof.

21. The method of claim 17, wherein the method further comprises administering a second therapeutic.

22. The method of claim 21, wherein the second therapeutic is an antibiotic.

23. A method of inhibiting the growth of or killing a bacterial cell, wherein the method comprises contacting the bacterial cell with at least one compound of claim 1 or a composition thereof.

24. A method of biosynthesizing a compound comprising the amino acid sequence (X^{A})ₐG(X^{B})_{b}L(X^{C})_{c}W(X^{D})_{d}, wherein the method comprises:
a) providing a nucleic acid to a host, wherein the nucleic acid encodes the amino acid sequence (X^{A})ₐG(X^{B})_{b}L(X^{C})_{c}W(X^{D})_{d} or a fragment thereof;
b) incubating the host in a growth medium; and
c) isolating the compound from the host or the growth medium,
wherein each occurrence of X^{A}, X^{B}, X^{C}, and X^{D} is independently selected from the group consisting of a natural amino acid, functionalized natural amino acid, unnatural amino acid, functionalized unnatural amino acid, and any combination thereof; and
wherein each occurrence of a, b, c, and d is independently an integer from 0 to 100.


SEQUENCE LISTING (47 sequences)

SEQ ID NO: 1 - skipped

SEQ ID NO: 2 - protein, 12 aa, unidentified
SNSGFLOENWOV

SEQ ID NO: 3 - protein, 12 aa, unidentified
TRSGFLREQWIT

SEQ ID NO: 4 - protein, 12 aa, unidentified
VRSGFLREVWGA

SEQ ID NO: 5 - protein, 12 aa, unidentified
VRSGFLRNVWGA

SEQ ID NO: 6 - protein, 12 aa, unidentified
TRSGFLRSTWIG

SEQ ID NO: 7 - protein, 12 aa, unidentified
VRVGPLREYWGA

SEQ ID NO: 8 - protein, 12 aa, unidentified
VERGPLXEYWGA

SEQ ID NO: 9 - protein, 14 aa, unidentified
SSSGWLTNTWFTGS

SEQ ID NO: 10 - protein, 12 aa, unidentified
SNSGYLVNTWFS

SEQ ID NO: 11 - protein, 6 aa, unidentified
GPLQEY

SEQ ID NO: 12 - protein, 7 aa, unidentified
SSSGWLT

SEQ ID NO: 13 - protein, 7 aa, unidentified
FXIGFLL

SEQ ID NO: 14 - protein, 18 aa, unidentified
XGALAXAXXXWLWLWLWG

SEQ ID NO: 15 - protein, 16 aa, unidentified
XGALAXXXWLWLWLWG

SEQ ID NO: 16 - protein, 14 aa, unidentified
VGALAXXXWLWLWG

SEQ ID NO: 17 - protein, 6 aa, unidentified
GLLVXS

SEQ ID NO: 18 - protein, 20 aa, unidentified
GXLEAEVXQXXAAAXQXAXE

SEQ ID NO: 19 - protein, 12 aa, unidentified
SNSGYLVNTWFS

SEQ ID NO: 20 - protein, 12 aa, unidentified
SNSGYLVDTWFS

SEQ ID NO: 21 - protein, 9 aa, unidentified
SSSGWLTNT

SEQ ID NO: 22 - protein, 19 aa, unidentified
XGXNXXXGXLAVVLXXXXX

SEQ ID NO: 23 - protein, 11 aa, unidentified
VGPLQEYGABA

SEQ ID NO: 24 - protein, 6 aa, unidentified
GPLQEY

SEQ ID NO: 25 - protein, 14 aa, unidentified
XLXXGDXXATGXLG

SEQ ID NO: 26 - protein, 17 aa, unidentified
PXFLXXLGXLAXXNXXL

SEQ ID NO: 27 - skipped

SEQ ID NO: 28 - protein, 11 aa, unidentified
XAXGFPXWGLL

SEQ ID NO: 29-31 - skipped (3 entries)

SEQ ID NO: 32 - protein, 23 aa, unidentified
XRXXASXLXFPGXLXLXXXXPXX

SEQ ID NO: 33 - protein, 4 aa, unidentified
HSTH

SEQ ID NO: 34 - protein, 8 aa, unidentified
PAAAGGGQ

SEQ ID NO: 35 - protein, 4 aa, unidentified
GVAA

SEQ ID NO: 36 - protein, 7 aa, unidentified
FSNGGXX

SEQ ID NO: 37 - protein, 14 aa, unidentified
VRSGFLRNVWGABA

SEQ ID NO: 38 - protein, 12 aa, unidentified
TRSGFLRSTWIG

SEQ ID NO: 39 - protein, 14 aa, unidentified
VRSGPLRNYWGABA

SEQ ID NO: 40 - protein, 5 aa, unidentified
GAGXS

SEQ ID NO: 41 - protein, 7 aa, unidentified
LXSNXXD

SEQ ID NO: 42 - protein, 7 aa, unidentified
XFVSGGA

SEQ ID NO: 43 - protein, 8 aa, unidentified
FELVXSXG

SEQ ID NO: 44-47 - skipped (4 entries)